SEQUENCE LISTING (16 sequences)

SEQ ID NO: 1 - DNA, 20 bp, Homo sapiens
aagcaaatggccaatgagat

SEQ ID NO: 2 - DNA, 27 bp, Homo sapiens
atctaaacagttacaaaacagatgtgc

SEQ ID NO: 3 - DNA, 21 bp, Homo sapiens
catcgatacctgcagcatctt

SEQ ID NO: 4 - DNA, 20 bp, Homo sapiens
tgtcttgctgccgtgtctta

SEQ ID NO: 5 - DNA, 20 bp, Homo sapiens
ttggcagtggactcagaaag

SEQ ID NO: 6 - DNA, 22 bp, Homo sapiens
tcagaagcagcaaaaatactgg

SEQ ID NO: 7 - DNA, 20 bp, Homo sapiens
ccgcgtggaatgtgagtgtg

SEQ ID NO: 8 - DNA, 23 bp, Homo sapiens
tgctgttcgcatatcacccattt

SEQ ID NO: 9 - DNA, 20 bp, Homo sapiens
tgcctgcccattggagagaa

SEQ ID NO: 10 - DNA, 23 bp, Homo sapiens
ttttcggatgctgtttacccatt

SEQ ID NO: 11 - DNA, 27 bp, Homo sapiens
tgttgtgattatctttttgtggtgtga

SEQ ID NO: 12 - DNA, 20 bp, Homo sapiens
gctggacaaccgtgggaaaa

SEQ ID NO: 13 - DNA, 60 bp, Homo sapiens
gttggctgatggcttttagcttgagccccaacagtgtgacttcatacaaggcaatttctt

SEQ ID NO: 14 - DNA, 60 bp, Homo sapiens
cctctggacaagggagggctttgcattcatgagggcttccactgtgctgcctcctcttaa

SEQ ID NO: 15 - DNA, 60 bp, Homo sapiens
tagaacgaagataagcaaactacaaaccaggaaaatgaaggggttgaagaagtgacctgc

SEQ ID NO: 16 - DNA, 60 bp, Homo sapiens
gcagagatccacgaggtattgagagcaacgcggaaaatagtagtgaaccctgtaaaaatc

(19) United States
(12) Patent Application Publication
(10) Pub. No.: US 2010/0105564 A1
(43) Pub. Date: Apr. 29, 2010

(54) Stroma Derived Predictor of Breast Cancer

(75) Inventors: Morag Park, Montreal (CA); Michael Hallett, Outremont (CA); Greg Finak, Montreat (CA); Svetlana Sadekova, Mountain View, CA (US)

Correspondence Address: DANN, DORFMAN, HERRELL & SKILLMAN, 1601 MARKET STREET, SUITE 2400, PHILADELPHIA, PA, 19103-2307, US

(73) Assignee: MCGILL UNIVERSITY, Montreal, QC (CA)

(21) Appl. No.: 12/441,280

(22) PCT Filed: Sep. 17, 2007
(86) PCT No.: PCT/CA07/01647

§ 371 Date: Oct. 22, 2009

Related U.S. Application Data
(60) Provisional application No. 60/825,831, filed on Sep. 15, 2006.

(51) Int. Cl. C40B 30/00; C12Q 1/68; G01N 33/53; C40B 40/06; G06F 19/00

(52) U.S. Cl. 506/7; 435/6; 435/7.1; 435/7.92; 506/16; 702/19

(57) ABSTRACT

The invention provides methods and compositions for use in the diagnosis and management of cancer, particularly breast cancer. The invention utilizes differential gene expression profiles in tumor associated stroma and normal stroma to compile a stroma derived prognostic predictor that classifies breast cancer patients according to clinical outcome. The application provides nucleic acids, antibodies, microarray chips and kits for use with the methods described in the application.

### FIELD OF THE INVENTION

The application relates to cancer and particularly to methods, compositions and kits for classifying patients with breast cancer according to clinical outcome.

### BACKGROUND OF THE INVENTION

Breast cancer is a major cause of morbidity and mortality in Western countries¹. Although disease-related mortality has declined due to earlier diagnosis and adjuvant therapies, identification of patients at increased risk of recurrence, targeting them for more aggressive systemic therapy, remains a significant challenge. One of the challenges is still to identify patients at risk of relapse and the desire to not overtreat. Options for advanced disease are limited. Recent technological advances now permit the systematic genomic characterization of tumors, enhancing our understanding of cancer causes and progression²⁻⁴. Gene expression signatures have been identified that classify breast tumors into subtypes exhibiting distinct expression profiles and associated with specific clinical outcomes⁴. Transcriptional signatures have been identified for estrogen receptor (ER)-positive (luminal), HER2-positive (ERBB2-amplified), and ER/PR/HER2-negative (basal) breast cancer⁴. Predictors of metastasis in breast cancer are becoming available for use in the clinic^{2,5}. Such prognostic gene expression signatures and predictors have generally been derived from tissues that include both tumor and stroma. Although some investigators have isolated and analyzed specific cell types or examined stroma-based gene expression signatures from cell culture experiments⁶⁻¹¹, most have used whole tissue consisting of tumor cells and the surrounding tissue environment, where samples with <50% tumor cells are generally excluded^{3,4,12}.

Gene expression in isolated tumor stroma from clinical breast cancer samples has not been examined; therefore, it is important to elucidate the specific contribution of stroma to tumor progression. The tumor microenvironment plays an important role in cancer initiation and progression^{13,14}. However, the exact mechanisms involved are not yet fully understood¹⁵⁻¹⁷.

There is thus a need for a new method or system to predict outcome recurrence for patients with cancers such as breast cancer, with greater accuracy, ease and convenience. The present invention seeks to meet this and related needs.

### SUMMARY OF THE INVENTION

The present inventors have used laser capture microdissection (LCM) to isolate tumor-associated and matched normal stroma from human breast cancer cases and performed microarray analyses to identify gene expression signatures or profiles associated with clinical outcome. From this, the inventors have developed a multivariate stromal derived prognostic predictor (SDPP) by ranking the independent predictive strength of each gene in the reference expression profile and identifying SDPP gene sets that are useful for predicting outcome in cancer patients.

In one aspect, the present application concerns the identification of a set of genes in tumor stroma that are predictive of the outcome of cancer in breast cancer patients. These genes include pro-angiogenic and hypoxia-related factors, as well as T-cell markers, the combination of which is predictive of recurrence. The set of genes may be used to develop clinical tests to identify patients at risk of developing recurrence or likely to have a poor prognosis. They may also serve as targets for combination therapeutics.

Accordingly, the present application provides a method for identifying a gene expression signature or profile of genes expressed in tumor associated stroma that is associated with, and useful for, predicting clinical outcome in cancer patients. A subset of the genes of the gene reference expression profile which is associated with disease outcome, is useful for predicting clinical outcome in a cancer patient. The method is useful for cancer types that comprise tumor associated stroma.

In another aspect, the application provides, a method of predicting clinical outcome in a breast cancer patient using a stroma derived prognostic predictor (SDPP), comprising the steps of comparing expression levels of a plurality of genes of a SDPP gene set in a sample of the patient to a reference expression profile of the genes, wherein the reference expression profile is associated with clinical outcome, and predicting clinical outcome, wherein clinical outcome is predicted according to the similarity of the expression level to the reference expression profile associated with the clinical outcome. In one embodiment the breast cancer is HER2 positive. In another embodiment the breast cancer is ER positive.

The application further provides in one embodiment, a method of predicting clinical outcome in a breast cancer patient comprising the steps of obtaining for a plurality of genes of a SDPP gene set in a sample of the patient, an expression level for the genes, comparing the expression level of the genes to a reference expression profile of the genes, wherein the reference expression profile is associated with a clinical outcome, and predicting clinical outcome, wherein clinical outcome is predicted according to the similarity of the expression level to the reference expression profile associated with the clinical outcome. The clinical outcomes in one embodiment are, good outcome, mixed outcome and poor outcome.

The present application also provides methods of determining prognosis wherein the prognosis comprises a good prognosis, a mixed prognosis, or a poor prognosis. The SDPP predicts clinical outcome or prognosis independently of standard clinical prognostic factors and previously published predictors and has increased accuracy with respect to previously published predictors.

In one embodiment, the application provides a method for determining prognosis in a breast cancer patient, comprising classifying the patient as having a good prognosis, a mixed prognosis or a poor prognosis comprising:
a) detecting gene expression of at least 3 genes of a stroma derived prognostic predictor (SDPP) gene set in a sample taken from the patient;
b) correlating the gene expression levels of the at least 3 genes with a disease outcome class, the class being good prognosis, poor prognosis or mixed prognosis.

In another embodiment the application describes a method for predicting disease outcome in a breast cancer patient, comprising:
a) obtaining an expression level of at least 3 genes of the SDPP gene set in a sample of the patient;
b) comparing the expression level of the genes in the sample to a reference expression profile for the genes in the SDPP gene set; and
c) predicting a good, mixed or poor prognosis disease outcome in the patient;
   wherein the reference expression profile of the at least 3 genes in the SDPP gene set correlates with a disease outcome class, the class being either a good prognosis, a mixed prognosis or a poor prognosis and wherein disease outcome is predicted according to the statistical probability of falling within the class defined by the reference expression profile of the at least 3 genes in the SDPP gene set.

In another embodiment, the application describes a method of diagnosing poor prognosis breast cancer comprising:
a) obtaining an expression level of at least 3 genes of a SDPP gene set in a sample of a subject;
b) comparing the expression level of the genes to a reference expression profile of corresponding genes in the SDPP gene set;
   wherein the reference expression profile of the at least 3 genes in the SDPP gene set correlates with a poor prognosis class and wherein the subject is diagnosed to have the poor prognosis according to the statistical probability of falling within the poor prognosis class.

An aspect provides a method of predicting the probability of cancer recurrence in a breast cancer patient. Accordingly in one embodiment the application provides a method for predicting recurrence in a breast cancer patient wherein a good prognosis predicts recurrence free survival of the patient, a poor prognosis predicts recurrence or non-survival, and a mixed prognosis predicts either recurrence free survival, or recurrence and/or non-survival comprising:
a) obtaining an expression level of at least 3 genes of a SDPP gene set in a sample of a patient;
b) comparing the expression level of the genes to a reference expression profile for corresponding genes in the SDPP gene set; and
c) predicting recurrence, no recurrence or mixed recurrence and no recurrence in the patient;
   wherein the reference expression profile of at least 3 genes in the SDPP gene set correlates with a recurrence class, the class comprising one or more of either no recurrence, recurrence or mixed recurrence and no recurrence and wherein recurrence is predicted according to the statistical probability of falling within the recurrence class defined by the reference expression profile of the at least 3 genes in the SDPP gene set.

In one embodiment, the application provides a method of predicting the probability of cancer metastasis. In another embodiment, the application provides a method of diagnosing tumor subtype. Accordingly, the application provides a method for diagnosing a breast cancer sub-type in a subject having breast cancer wherein a good prognosis predicts a breast cancer subtype associated with recurrence free survival, a poor prognosis predicts a breast cancer subtype with recurrence or non-survival, and a mixed prognosis predicts a breast cancer subtype with either recurrence free survival, or recurrence and/or non-survival comprising the steps of:
a) obtaining an expression level of at least 3 genes of a SDPP gene set in a cancer sample of a subject; and
b) comparing the expression level of the genes to a reference expression profile of corresponding genes in the SDPP gene set; and
c) diagnosing the cancer sub-type;
   wherein the reference expression profile of the at least 3 genes in the SDPP gene set correlates with a cancer sub-type class, the class comprising one or more of a good, mixed or poor prognosis cancer sub-type and wherein the subject is predicted or diagnosed to have the good, mixed or poor prognosis cancer subtype according to the statistical probability of falling within the class defined by the reference expression profile of the at least 3 genes in the SDPP gene set.

Diagnosing tumor subtype is important for a variety of applications including assigning treatment and assigning patients to appropriate clinical trials.

Accordingly another aspect relates to a method of assigning or selecting a treatment or therapy for a breast cancer patient. In one embodiment the application provides a method for classifying a breast cancer wherein a good prognosis classifies a breast cancer class in a recurrence free survival class, a poor prognosis classifies a breast cancer in a recurrence or non-survival class, and a mixed prognosis classifies a breast cancer in either recurrence free survival, or recurrence and/or non-survival class comprising:
a) obtaining an expression level of at least 3 genes of a SDPP gene set in a cancer sample of a patient;
b) comparing the expression level of the genes to a reference expression profile for the genes in the SDPP gene set; and
c) classifying the cancer as a good mixed or poor prognosis cancer;
   wherein the reference expression profile of the at least 3 genes in the SDPP gene set correlates with a cancer class, the class comprising one or more of a good, mixed or poor prognosis cancer and wherein the subject is predicted or diagnosed to have the good, mixed or poor prognosis cancer according to the statistical probability of falling within the class defined by the reference expression profile of the at least 3 genes in the SDPP gene set.

In one embodiment, method of selecting or assigning a treatment to a breast cancer patient comprises
a) classifying the cancer according to a method described in the application; and
b) assigning an appropriate treatment according to the cancer class.

In one embodiment, a method for optimizing treatment is provided. In another embodiment, a method for monitoring treatment is provided. In yet a further embodiment, a method of assigning a subject to or selecting a subject for a clinical study is provided. Accordingly the application describes a method of assigning a breast cancer patient to a clinical trial comprising:
a) classifying the cancer according to a method described in the application; and
b) assigning the patient to a clinical trial for the cancer class.

Another aspect relates to integration of the SDPP predictor with other predictors and signatures. Combining the SDPP with other known predictors and signatures improves clinical outcome prediction such as the prediction of metastases. The predictors are combined in one embodiment using a graphical modeling approach. In one embodiment the SDPP is combined to construct a predictor of metastasis.

The application provides a number of SDPP gene sets comprising a plurality of genes that are useful with the methods described in the application. In one embodiment the SDPP gene set comprises at least 3 genes, 4-5 genes, at least 5 genes, 6-10 genes, 11-14 genes, 15 genes, 16-18 genes, 19 genes, 20-25 genes, 26 genes, 27-30 or more than 30 genes of the genes listed in Tables 3-6 and 9-11. In another embodiment, the application involves the use of a sub-set of genes such as 20 genes that are expressed in breast tumor stroma for diagnostic and possible therapeutic purposes.

One aspect of the application is a composition comprising a plurality of nucleic acid sequences, wherein each nucleic acid sequence hybridizes to an RNA product of a gene of a SDPP gene set or a nucleic acid sequence complementary to the RNA product, wherein the composition is used to detect the level of expression of at least 2 genes of a SDPP gene set. The application also relates to specific primers and probes.

Another aspect of the application is a composition comprising a plurality of 2 or more binding agents for example, isolated polypeptides, where each binding agent binds to a polypeptide product of a gene of a SDPP gene set described in the application.

The application also provides in one aspect a method of identifying agents for use in the treatment of cancer. In one embodiment the method comprises identifying an agent that inhibits expression of one or more hypoxia response genes implicated in poor prognosis. In another embodiment, the method comprises identifying an agent that inhibits expression of one or more Th2 response genes associated with poor prognosis. In a further embodiment, the method comprises identifying an agent that inhibits expression of one or more angiogenesis genes associated with poor prognosis. In yet a further embodiment, the method comprises identifying an agent that inhibits expression of at least two genes selected from the group consisting of hypoxia response genes, Th2 response genes and angiogenesis genes associated with poor prognosis.

The application also includes kits comprising nucleic acids and polypeptides described herein, that are useful for detecting expression levels of SDPP gene set gene products. In one embodiment, the kit comprises components for multiplex PCR.

The application further includes arrays that are useful for detecting SDPP gene set expression levels. In one embodiment, the array is a microarray. In a further embodiment, the array is a DNA array. In another embodiment, the array is a tissue array.

The application further includes computer systems, computer readable mediums and computer program products for implementing the methods described in the application.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

An embodiment of the application will now be described in relation to the drawings in which:

FIG. 1 is a series of charts and graphs illustrating class discovery of tumor associated stroma. (a) is a flow chart outlining principal steps in the construction of the SDPP; (b) is a graph demonstrating class discovery in tumor-associated stroma samples over a basis set of the 200 most variable genes observed from matched normal vs. tumor-associated stroma gene expression data. Clusters in the tree are labeled with the percentage of times they were observed in 1000 bootstrap iterations. Clinical characteristics of each tumor sample are presented in the shaded boxes below each sample, with a shaded box representing a positive status. Poor outcome is defined as dead of disease or alive with disease as of last follow up. Significant associations of each cluster with clinical characteristics are presented below the relevant cluster; (c) is a graph of Kaplan-Meier survival curves for patients belonging to the good outcome (dotted line), poor outcome (dashed line), and mixed outcome (solid line) clusters in FIG. 1(b); (d) is a table presenting Multivariate Cox regression (MVCR) for the clusters depicted in FIG. 1 (b).

FIG. 2 is a series of microarray data plots illustrating class distinction of tumor stroma. (a) is a plot illustrating hierarchical clustering of tumor-associated stroma samples using the 163 genes differentially expressed between the good-, poor-, and mixed-outcome clusters of FIG. 1a. Gene clusters are labeled with significance from bootstrap analysis, and color bars to represent the three gene clusters described in the text. Heatmap colors represent mean-centered fold-change expression in log-space; (b) is a graph of Kaplan-Meier curves for each of the three clusters; (c) is an expanded view of the genes expressed predominantly in patients of the good outcome cluster; (d) is a plot illustrating genes expressed predominantly in patients of the poor outcome cluster; (e) is a plot illustrating genes expressed predominantly in patients of the mixed outcome cluster. (*) denotes the gene is a member of the SDPP gene set.

FIG. 3 is a series of graphs and plots illustrating performance of the SDPP. (a) is a Receiver-operator-characteristic (ROC) curve for the SDPP applied to tumor stroma samples, showing the true positive and false positive rate, as well as the AUC. The AUC corresponds to the probability of the SDPP assigning a higher score to a randomly selected positive example than a randomly selected negative example; (b) is a heatmap showing the predictions made by the SDPP in the stroma data set. Samples are ordered by the probability of membership in each of the three classes, while genes are arranged by hierarchical clustering. Gene cluster color-codes are as in FIG. 2a. Heatmap colors represent mean-centered fold-change expression in log-space; (c) is a graph of Kaplan-Meier curves for the three patient groups identified by the SDPP.

FIG. 4 is a series of plots and graphs illustrating performance of the SDPP in previously published breast cancer gene expression data sets. (a) is a plot illustrating predictions of good, poor, and mixed outcome for patients in the NKI data set using the SDPP. Samples are ordered by their score from the SDPP, genes by hierarchical clustering. Tick marks below the heatmap represent metastasis or relapse events; (b) is a graph illustrating overall survival and (c) is a graph illustrating time to metastasis of patients predicted as good, poor, and mixed-outcome in the NKI data set. Solid lines are survival curves for the complete data set; dashed lines, survival curves for the HER2-positive patient subset. Relative risks, median survival, and p-values are shown for the complete data, and in brackets for the HER2-positive subset; (d) is a plot illustrating predictions of good, poor, and mixed outcome for patients in the Wang et al. data set using the SDPP. Samples and genes are ordered as above. Tick marks below the heatmap represent relapse events; (e) is a graph illustrating relapse-free survival (RFS) of patients belonging to the good, poor and mixed-outcome groups in the Wang et al. data set. Solid lines, dashed lines and relevant values are depicted as described above.

FIG. 5 is a series of immunohistochemical sections and Q-RTPCR plots demonstrating the validation of elements of the SDPP. (a) Immunostaining for CD8A in patients 1) E1056, 2) E1227, 3) E1897, all of whom recurred, and 4) E1228, 5) E1527, and 6) E1277, all currently disease-free. (b) Immunostaining for CD3Z in patients 1) E1879 and 2) E1056, both of whom recurred, and 3) E1277, and 4) E1751, both currently disease-free. (c) Immunostaining for SPP1 (osteopontin) in patients 1) E1808, and 2) E1792, both of whom recurred, as well as 3) E1751 and 4) E1527, both currently disease-free. Scale bar, 250 microns. Inset is a low power view. (d) Regression of fold change in array expression vs. fold change in RTPCR concentration for selected genes identified in the stroma predictor. Fold changes are expressed relative to the lowest-expressing sample observed in the array data. The p-value is the significance of the slope term in the regression model.

FIG. 6 is a series of ROC curves for training the SDPP. (a) The average AUC for a variety of predictors trained on tumor-associated stroma plotted as a function of the number of genes in the predictor. The “optimal” predictor (highlighted in green) was chosen to maximize the AUC and contained 26 genes. b)-e) ROC curves for the optimal 26-gene SDPP trained on: (b) tumor stroma, (c) tumor epithelium (d) normal stroma (e) normal epithelium. Error bars show the standard error of the ROC curves based on 50 cross validation runs.

FIG. 7 is a graph illustrating selected Gene Ontology (GO) terms over-represented by the genes expressed in the predicted good-outcome (left panel) and poor-outcome (right panel) patient clusters.

FIG. 8 is a series of plots and immunostained sections illustrating differential expression of selected genes and CD31. (a) Differential expression between clusters of FIG. 2a for genes specifically linked to hypoxia, angiogenesis, and Th1- or Th2-type immune responses in the tumor-associated stroma. (b) Box plots, (c) Tukey's HSD test, and (d) levels of immunostaining for CD31 in selected samples from the green, red, and blue patient clusters in FIG. 2a. Bars in (c) represent 95% family-wise confidence intervals. (d) 1-4: CD31 immunostaining of sections from patients in (1-4) the mixed-outcome cluster; (5-8) the good-outcome cluster 2; and (9-12) the poor-outcome cluster. Scale bar, 1.2 mm.

FIG. 9 is a series of graphs and tables showing evaluation of SDPP performance other data sets (a) Multivariate Cox regression (MVCR) for overall survival in the complete NKI¹⁸ data set. (b) MVCR for relapse-free survival in the complete Wang et al.¹² data set. (c) HER2 and ER status composition of the good, poor, and mixed-outcome samples identified by the SDPP in the NKI data set. (d) Fraction of the good, poor and mixed-outcome patients identified by the SDPP in the NKI data that are also identified as either hypoxic or non-hypoxic by a hypoxia-associated transcriptional response¹⁹. (e) Performance measures of the SDPP and 70-gene predictors in the NKI data set for predictions of good and poor outcome. (f) Structure of the Bayes' classifier trained to predict metastasis from combinations of multiple predictors. Each node represents a random variable, while arcs in the graph represent dependencies between random variables. The direction of the arc indicates that the random variable with the incoming arc depends upon the random variable with the outgoing arc. (g) Combinatorial prediction in the NKI data set. The posterior probability of metastasis was calculated from the Bayes' classifier of metastasis trained on predictions of good and poor outcome for the SDPP, 70-gene predictor, wound signature, and hypoxia signature. The probability of metastasis is computed for different combinations of poor and good outcome predictions from each signature. A black box indicates a poor outcome prediction from a signature, an empty box indicates a good outcome prediction from a signature, and a grey box indicates that information from that predictor was not used. Grey circles below the dashed line highlight predictions where the good-outcome SDPP was used, while grey circles above the dashed line highlight predictions where the poor-outcome SDPP was used. The grey dotted line identifies the prior probability of metastasis for the case where not predictor information is available.

FIG. 10 is a plot illustrating a cluster of tumor stroma that is associated with patients with poor outcome.

FIG. 11 is a plot demonstrating clusters in the tumor expression data.

FIG. 12 is a graph demonstrating prognostic ability in stroma and epithelium.

FIG. 13 is a series of Kaplan Meier survival graphs.

FIG. 14 is a microarray data plot.

### DETAILED DESCRIPTION OF THE INVENTION

It is increasingly evident that breast cancer outcome is strongly influenced by signals emanating from tumor-associated stroma. However, little is known about how gene expression changes in this tissue affect tumor progression.

The inventors are the first to provide a predictor of clinical outcome in patients with breast cancer based on normal and tumor-associated stroma cell expression profiles. The inventors have compared gene expression profiles from laser capture-microdissected tumor-associated versus matched normal stroma, and have derived transcriptional or reference expression profiles strongly associated with clinical outcome. Based on the outcome associated profiles derived from tumor associated stroma, the inventors have developed a prognostic tool for predicting clinical outcome. Disclosed herein is a stroma-derived prognostic predictor (SDPP) that provides new information to stratify disease outcome in breast cancer patients, independent of standard clinical prognostic factors and previously published predictors. The SDPP selects poor-outcome patients from multiple clinical subtypes, including lymph node-negative patients, and predicts outcome in multiple published expression data sets generated from whole tumor tissue. The SDPP has increased accuracy with respect to previously published predictors and prognostic accuracy increases upon predictor integration. Genes represented in the SDPP gene sets reveal the strong prognostic capacity of differential immune responses as well as angiogenic and hypoxic responses.

Accordingly, in one embodiment, the application provides a stroma derived prognostic predictor (SDPP). The SDPP compares the expression level of 5 or more genes of a SDPP gene set in a sample of a breast cancer patient to the reference expression profile of the genes, the reference expression profile being associated with a disease outcome class, and predicts disease outcome according to the probability of falling within the disease outcome class defined by the reference expression profile of the SDPP genes.

As used herein “SDPP” means stroma derived prognostic predictor and refers to a multivariate predictor or classifier generated from comparing gene expression in tumor associated versus normal stroma and identifying a reference expression profile of genes and/or gene sets associated with and predictive of a clinical outcome class, the classes being good, mixed and poor outcome. The SDPP predictor includes the correct weighting of genes. The SDPP provides a number of “SDPP gene sets” and the correct weighting of each gene in the gene set. The SDPP is useful for a variety of methods including methods for predicting clinical outcome, recurrence and metastasis, classifying and stratifying patients and tumors according to clinical outcome, diagnosing cancer subtype and/or providing a prognosis wherein the prognosis is good, mixed (alternatively referred to as uncertain) or poor. The SDPP gene sets are also useful for assigning, optimizing and monitoring treatment and assigning patients to clinical trials. The SDPP is useful in one embodiment for assigning, optimizing and monitoring treatment and assigning patients to clinical trials for HER2 positive cancers.

As used herein “SDPP gene set” means a set of genes identified as predictive of outcome using a classifier such as a naïve Bayes classifier, whose expression profile is associated with and predictive of a clinical outcome class. The gene sets were identified using a method wherein genes of a gene signature of tumor associated stroma subtypes were ranked according to their independent prognostic ability (Table 3) and then sets of incrementally larger gene sets from the ordered list were assessed using a multivariate naïve Bayes classifier to identify SDPP gene sets that are predictive of clinical outcome.

In one embodiment, the SDPP gene sets comprise genes listed in Tables 3-6 and 9-11, which are useful for predicting disease or clinical outcome. In a preferred embodiment the SDPP gene set comprises gene sets listed in Tables 9-11.

The inventors have shown that prediction is also accomplished using a subset of genes in a SDPP gene set. By way of example, the inventors demonstrate that a subset of 15 of the 26 genes in the SDPP gene set provided in Table 9 (which 15 genes are listed in Table 11) is useful for predicting clinical outcome in one dataset (the NKI dataset) and a subset of 19 of the 26 genes in the SDPP gene set provided in Table 9 (which 19 genes are listed in Table 11) is useful for predicting clinical outcome in another dataset (the Wang et al.¹² dataset). Accordingly in one embodiment, the gene set comprises a gene set listed in Table 11.

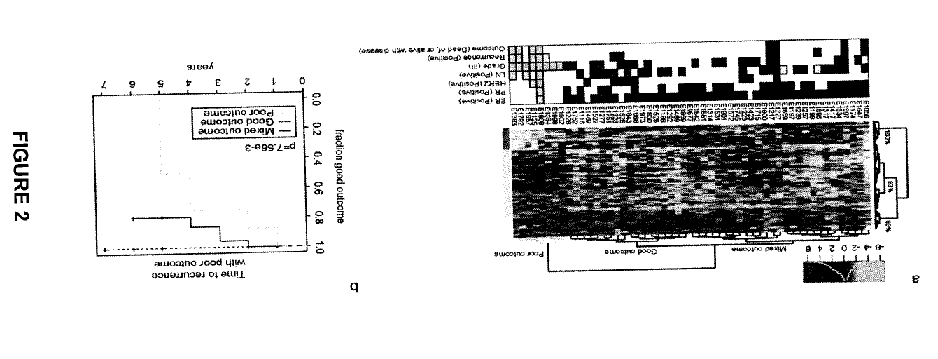

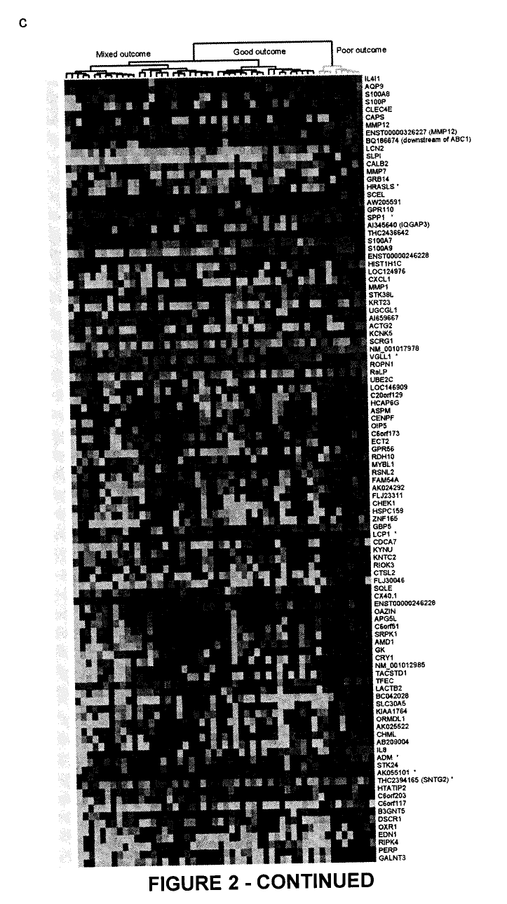

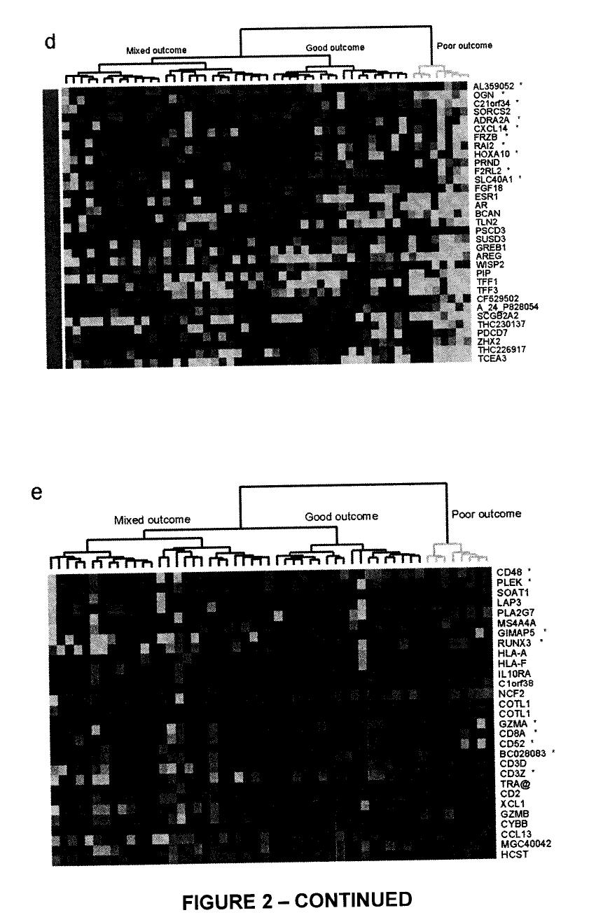

In addition, a number of different SDPP gene sets were found to be predictive of outcome. Gene sets comprising as few as 3 genes are useful for the methods described in the application. The gene sets or subsets thereof used in the method described herein include at least one gene from each of three gene cluster groups identified (FIG. 2a). One gene cluster comprises genes predominantly elevated in the poor outcome class and includes genes associated with an angiogenic response and hypoxia response. A second comprises genes predominantly expressed in the good outcome class and the third comprises genes expressed in both the good and mixed outcome class. The SDPP gene sets useful for predicting clinical outcome comprise at least one gene from each of the identified gene clusters. For example a SDPP gene set in one embodiment comprises at least one gene having a reference expression profile associated with good outcome, at least one gene having a reference expression profile associated with mixed and good outcome and at least one gene having a reference expression profile associated with poor outcome. In one embodiment the SDPP gene set comprises at least one group 1 gene, at least 1 group 2 gene; and at least one group 3 gene, of Table 10. Accuracy of prediction is increased by including additional SDPP gene set genes. In one embodiment the gene set comprises at least 3, 4-5, at least 5, 6-10, 11-14, at least 15, 16-18, 19, 21-25, 26 or at least 26 of the genes listed in Tables 3-6, and/or 9-11. In one embodiment the gene set comprises at least 3 genes listed in Table 10 comprising at least one group 1 gene, at least 1 group 2 gene and at least one group 3 gene. In another preferred embodiment, the gene set comprises the genes listed in Table 9. The genes listed in Table 9 comprise the genes identified as the optimal predictor.

As used herein “clinical outcome”, alternatively referred to as “disease outcome”, also as “prognosis” is a patient class defined by a reference expression profile of a SDPP set comprising at least 3 genes. The clinical outcome, or prognosis means as used herein an indication of disease progression and includes an indication of likelihood of recurrence, metastasis, death due to disease, tumor subtype or tumor type. In one embodiment the clinical outcome class includes a good outcome, a poor outcome and a mixed outcome class. The clinical outcome class in another embodiment comprises a good prognosis, a mixed prognosis and/or a poor prognosis. A “good outcome” or a “good prognosis” as used herein refers to an increased likelihood of disease free survival for at least 60 months A “poor outcome” or “poor prognosis” as used herein refers to an increased likelihood of relapse, recurrence, metastasis or death within 60 months. A mixed outcome or mixed prognosis as used herein refers to a class that comprises both good outcome or prognosis and poor outcome or prognosis patients.

As used herein “expression level” of a gene of a SDPP gene set refers to the quantity of gene product produced by the gene in a sample of a patient wherein the gene product can be a transcriptional product or a translated transcriptional product. Accordingly the expression level can pertain to a nucleic acid gene product such as RNA or cDNA or a polypeptide gene product. The expression level is derived from a patient sample. The expression level in certain embodiments is detected using methods known in the art and described herein. As the inventors have shown the expression level of genes of a SDPP gene set may also be extracted from data comprising expression levels of a subset of SDPP genes. For example the expression levels is optionally obtained from data derived from a patient sample for other tests. Accordingly, in one embodiment the expression level of SDPP genes is obtained from a data set comprising values for the expression of at least 3 genes of a SDPP gene set. In a preferred embodiment the genes comprise genes from the SDPP gene set listed in Tables 9-11.

A “reference expression profile” optionally referred to as an “expression profile” as used herein refers to the expression signature of SDPP genes or a gene set associated with a clinical outcome in a breast cancer patient. The reference expression profile is identified using one or more samples comprising tumor associated stroma wherein the expression is similar between related samples defining an outcome class and is different to unrelated samples defining a different outcome class such that the reference expression profile is associated with a particular clinical outcome. The reference expression profile is accordingly a reference profile or reference signature of the expression of SDPP gene set genes, the SDPP genes being genes listed in Tables 3-6 and 9-11, to which the expression levels of the corresponding genes in a patient sample are compared in methods for determining or predicting clinical outcome.

As used herein “sample” refers to any fluid, cell or tissue sample from a patient which can be assayed for gene expression levels, particularly genes differentially expressed in patients having or not having breast cancer. The sample comprises a cancer cell or cells or a tumor associated stroma cell or cells. Although the SDPP gene sets were identified using tumor associated stroma, the methods can be applied to tumor and/or tumor associated samples with or without stromal tissue. The inventors have shown that the SDPP is useful for predicting outcome using data derived from whole breast tumor tissue, containing tumor and stroma. As used herein, sample refers to a patient tumor or tumor associated sample. Tumor and cancer are herein used interchangeably. The sample is optionally a biopsy, a paraffin embedded section or material, a frozen specimen or fresh tumor tissue.

### Identifying Classes and Genes for Predicting Clinical Outcome

The application provides in one embodiment, a method to identify or discover classes according to the differential expression in tumor associated versus normal stroma. The inventors have conducted microarray experiments using tumor associated and normal stromal RNA samples and have identified the top 200 most variable genes across a group of breast cancer patients. Tumor stroma was clustered using these genes, identifying or discovering good outcome, mixed outcome and poor outcome classes, and the significance of the clusters was assessed by bootstrapping. A person skilled in the art will recognize that other numbers of most variable genes can be used. For example the top 50, 51-100, 101-200, 201-300 or more genes can be used.

“Class discovery” as used herein refers to a method of analyzing data such as microarray data to identify or discover reproducible classes or clusters that have similar behaviour or properties, within the data set.

In another embodiment the application provides a method of identifying informative genes, which are informative for predicting a class distinction. The inventors used pairwise class distinction to identify genes differentially expressed between the poor outcome, mixed outcome and good outcome classes. A reference expression profile for the outcome classes was derived. The class distinction in one embodiment is clinical outcome or prognosis. In other embodiments the class distinctions include among others disease recurrence, metastasis and tumor subtype.

“Class distinction” as used herein refers to a method of analyzing data such as microarray data that identifies features such as genes that distinguish between known classes. To construct the multivariate predictor, the inventors trained Bayes' classifiers to predict prognosis using a ranked gene reference expression profile of the recurrence positive stroma cluster. The inventors are the first to use tumor associated stroma to construct a multivariate predictor. A person skilled in the art will recognize that although breast cancer tissues were used to derive the predictor, other cancer types that involve stomal involvement can also be used to derive a predictor for the cancer type.

As mentioned, the inventors used breast cancer tissues to develop a multivariate predictor. Accordingly, the application also provides a stromal derived prognosis predictor (SDPP) which is a multivariate predictor of clinical outcome in breast cancer patients.

A number of SDPP gene sets were identified that are useful with the methods described in the application for predicting clinical outcome in a breast cancer patient. Comparison of the expression level of 5 or more genes of a SDPP gene set in a sample of a patient to the gene reference expression profile the 5 or more genes of the SDPP gene set associated with a clinical outcome permits prediction of a clinical outcome in the patient.

“Class prediction” as used herein refers to a method of classifying unknown samples into known classes. The stroma derived prognostic predictor disclosed herein provides a predictor for classifying disease outcome of cancer patients into good, poor and mixed classes.

Accurate prediction and/or diagnosis of disease outcome, tumor subtype, disease recurrence or metastasis is important for a number of reasons. Patients may be classified on the basis of clinical outcome which allows for example assigning or selecting appropriate treatment plans according to the aggressiveness of the particular disease subtype. It further provides additional information that is useful for assigning or selecting subjects for clinical trials. The efficacy of new therapeutic agents can therefore be assessed according to the particular profiles of the trial participants which can also provide for more appropriate treatment options according to the disease subtype.

Gene weighting is assigned using a probabilistic classifier such as a naïve Bayes classifier. A “naïve Bayes classifier” as used herein refers to a simple probabilistic classifier based on applying Bayes theorem. The naïve Bayes classifier is trained in a supervised setting.

As mentioned, the methods of constructing a stromal derived classifier or predictor and identifying stromal derived gene sets that are predictive of clinical outcome can be applied to any cancer wherein the tumor is associated with stroma and expression levels in tumor associated stroma and normal stroma can be detected.

In one embodiment the application describes a method for predicting the likelihood of recurrence or prognosis of breast cancer in a patient, said method comprising:
isolating normal stroma and epithelium as well as tumor stroma and epithelium from breast tissue samples;
identifying the top 200 most variable genes across all samples;
using LIMMA and SAM approaches to identify the genes differentially expressed between poor outcome tumor stroma subtypes and remaining tumor stroma samples;
using the set union of these approaches to derive expression profiles of tumor stroma with poor outcome; and
comparing said expression profiles with the expression profile of tumor stroma of the patient to determine the likeliness of recurrence or prognosis of breast cancer in the patient.

In another embodiment, the application describes a method for predicting the likelihood of recurrence or prognosis of breast cancer in a patient, said method comprising:
isolating normal stroma and epithelium as well as tumor stroma and epithelium from breast tissue samples;
identifying the top 200 most variable genes across all samples;
using LIMMA and SAM approaches to identify the genes differentially expressed between poor outcome tumor stroma subtypes and remaining tumor stroma samples;
using the set union of these approaches to derive expression profiles of tumor stroma with poor outcome; and
comparing said expression profiles with the expression profile of tumor stroma of the patient to determine the likeliness of recurrence or prognosis of breast cancer in the patient.

In a further embodiment the application describes a method for predicting the likelihood of recurrence or prognosis of breast cancer in a patient, said method comprising:
isolating normal stroma and epithelium as well as tumor stroma and epithelium from breast tissue samples;
identifying the top 20 most variable genes across all samples;
using LIMMA and SAM approaches to identify the genes differentially expressed between poor outcome tumor stroma subtypes and remaining tumor stroma samples;
using the set union of these approaches to derive expression profiles of tumor stroma with poor outcome; and
comparing said expression profiles with the expression profile of tumor stroma of the patient to determine the likeliness of recurrence or prognosis of breast cancer in the patient.

In a yet a further embodiment the application describes a method for predicting the likelihood of recurrence or prognosis of breast cancer in a patient, using a method of described in the application wherein the 20 genes are: GZMA, CD8A, BC028083, CD52, CD48, CD3Z, GIMAP5, F2RL2, SLC40A1, RAI2, OGN, C21orf34, adrA2A, HOXA10, SPP1, HRASLS, VGLL1, ADM, AK055101 and THC2394165.
A method of identifying a stroma derived predictor gene set comprising a plurality of genes whose expression profile is associated with disease outcome in a cancer patient comprising:
a) determining a gene expression level in a first sample comprising tumor associated stroma and in a second sample comprising normal stroma;
b) identifying at least 50 of the genes that vary most between the first and the second sample;
c) clustering the first sample according to the at least 50 most variable genes to identify clusters associated with a disease outcome, wherein the outcomes include at least good outcome and poor outcome;
d) identifying a gene set that comprises genes from each of the clusters that correlates with the disease outcome; and
e) determining whether the correlation is stronger than expected by chance;
wherein the stoma derived predictor gene set is the set of genes that correlates with disease outcome in the patient more strongly than expected by chance.

In another embodiment, the application describes a method of identifying a stroma derived predictor gene set consisting of a plurality of genes comprising:
a) comparing a gene expression level in a sample comprising tumor associated stroma to a sample comprising normal stroma;
b) sorting at least 50 genes by degree to which their expression in the sample comprising tumor associated stroma vary most from the sample comprising normal stroma;
c) identifying a gene set from the sorted genes that correlates with a disease outcome wherein the disease outcome is either a good prognosis, a mixed prognosis or a poor prognosis;
d) determining whether the correlation is stronger than expected by chance; and
e) displaying or outputting a result of steps a), b) c) or d) to a user, a computer readable storage medium, a monitor, or a computer that is part of a network;
   wherein the SDPP gene set is the set of genes that correlates with a disease outcome more strongly than chance.

### Cancers

The application provides a method for predicting clinical outcome in a breast cancer patient using SDPP. Different breast cancer disease subtypes are known in the art and the SDPP is optionally used to predict outcome in any breast cancer subtype. The breast cancer is optionally node negative or node positive, ER positive or ER negative, HER2 positive or HER2 negative, PR positive or PR negative, high grade or low grade, basal-like or luminal-like, or any combination of these six factors. The inventors have shown that the methods described in the application are useful for predicting disease outcome prior to node involvement in breast cancer patients. Accordingly, in one embodiment the application provides a method of predicting disease outcome in a node negative breast cancer patient. The inventors have further shown that the SDPP is useful for predicting good versus poor outcome in patients having ER positive and HER2 positive cancers. Accordingly, the application provides in one embodiment a method of predicting clinical outcome in a patient that has an ER positive breast cancer. In another embodiment, the methods are applied to a patient having an ER negative breast cancer. In another embodiment, the methods described in the application are applied to a patient with a HER2 positive breast cancer. In a further embodiment the methods described in the application are applied to a patient with a HER2 negative breast cancer.

As stromal changes accompany other cancers with stromal involvement, the methods of identifying a stroma derived predictor and of identifying a stromal derived gene set based on gene expression differences in tumor associated stroma versus normal stroma are applicable to different cancer types. “Cancer” as used herein refers to a group of diseases characterized by uncontrolled growth and spread of abnormal cells. Cancer and tumor are herein used interchangeably.

Accordingly, the application provides methods that are useful for identifying stromal derived predictor gene sets that are associated with clinical outcome in a cancer patient. In another embodiment the methods and stromal derived predictor gene sets described herein are useful for predicting disease outcome in a cancer patient or cancer subject. In one embodiment the cancer type is breast cancer. In another embodiment the cancer type is a colon cancer. In a further embodiment, the cancer type is a lung cancer. in other embodiments the cancer type is bladder, prostate or ovarian cancer.

### Nucleic Acid Compositions

One aspect of the application is a composition comprising a plurality of at least two isolated nucleic acid sequences. The isolated nucleic acids comprise sequences complementary to novel SDPP genes.

### SDPP Genes and Nucleic Acids

The application describes a number of SDPP genes and gene sets. In one aspect the application provides a SDPP gene set comprising two or more isolated nucleic acids corresponding to SDPP genes. In one embodiment the SDPP gene set comprises at least 2, 3, 4, 5, 6, 7-10 or more isolated nucleic acids corresponding to SDPP genes. In another embodiment the SDPP gene set comprises 11-14, 15, 16-18, 19, 20-25, 26, 27-29, 30-50, 50-100, 100-162, 163, 164-199 or 200 isolated nucleic acids. In another embodiment the SDPP gene set genes are selected from genes listed in Tables 2-5 and 9-11. In one embodiment, the SDPP gene set comprises a plurality of two or more isolated nucleic acid sequences listed in Tables 3-7 and 9-11

The SDPP gene sets also comprise a number of novel gene products that correlate with disease outcome. These include gene products which hybridize to probes THC2436642 (SEQ ID NO: 13), A_{—}24_P82805 (SEQ ID NO: 14), ENST0000024 (SEQ ID NO:15), and THC2269172 (SEQ ID NO:16) THC2436642 is a TIGR human consensus sequence identifier and corresponds to probe A_{—}32_P13533 with sequence GTTGGCTGATGG CTTTTAGCTTGAGCCCCAACAGTGTGACTTCATACAAGGCAATTTCTT (SEQ ID NO: 13). The sequence for A_{—}24_P82805 probe is CCTCTGGACAAGGGAGGGCTTTGCATTCATGAGGGCTTCCACTGTGC TGCCTCCTCTTAA (SEQ ID NO: 14). ENST00000246228 corresponds to probe A_{—}23_P366468 with sequence TAGAACGAAGATAAGCAAACTACAA ACCAGGAAAATGAAGGGGTTGAAGAAGTGACCTGC (SEQ ID NO: 15). THC2269172 corresponds to probe is A_{—}24_P936252 with sequence GCAGAGATCCACGAGGTATTGAGAGCAACGCGGAAAATAGTA GTGAACCCTGTAAAAATC (SEQ ID NO: 16) The provided names beginning with “A_” are the agilent probe ids The THC numbers are TIGR tentative human consensus sequence identifiers.

In one embodiment, the application provides an isolated nucleic acid comprising a polynucleotide sequence selected from the group consisting of:
a) a polynucleotide sequence complementary to of any one of SEQ ID NOS: 13-16;
b) a polynucleotide sequence having at least 70%, 80% or 90% sequence identity with a nucleic acid of a); and
c) a polynucleotide sequence that that hybridizes to SEQ ID NOS: 13-16 under stringent conditions.

The term “isolated nucleic acid sequence” as used herein refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded.

The term “hybridize” refers to the sequence specific non-covalent binding interaction with a complementary nucleic acid. One aspect of the application provides an isolated nucleotide sequence, which hybridizes to a RNA product of a gene of a SDPP gene set described in the application or a nucleic acid sequence which is complementary to an RNA product of a gene of a SDPP gene set described in the application. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The stringency may be selected based on the conditions used in the wash step. For example, the salt concentration in the wash step can be selected from a high stringency of about 0.2×SSC at 50° C. In addition, the temperature in the wash step can be at high stringency conditions, at about 65° C.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10[Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm-5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

The term “products of a gene of a SDPP gene set” as used herein refers to RNA and/or the polypeptide expressed by a gene of a SDPP gene set described in the application. In the case of RNA, it refers to RNA transcripts transcribed from a gene of a SDPP gene set described in the application. The term “RNA product” of the gene of a SDPP gene set described in the application as used herein includes mRNA transcripts, and/or specific spliced variants of mRNA. In the case of protein, it refers to proteins translated from the RNA transcripts transcribed from the genes of a SDPP gene set described in the application. The term “polypeptide product” of a gene of a SDPP gene set described in the application includes polypeptides translated from the RNA products of the gene of a SDPP gene set described in the application.

### Nucleic Acids, Primers and Probes

One aspect of the application provides, a composition comprising a plurality of two or more isolated nucleic acid sequences, wherein each isolated nucleic acid sequence hybridizes to:
a) a RNA product of a gene of a SDPP gene set; and/or
b) a nucleic acid sequence complementary to a),
   wherein the composition is used to detect the level of RNA expression level of two or more genes of a SDPP gene set.

In one embodiment, the composition comprises two or more genes of a gene set that are selected from those in Tables 3-7 and 9-11.

In another aspect, the application provides use of a collection of two or more isolated nucleic acid sequences are sets of specific primers. In one embodiment the nucleic acid sequences are the sequences as set out in Table 8. In another embodiment, the use comprises use of primers specific for one or more genes listed in Tables 3-6 and 9-11.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. The term “SDPP gene specific primer” as used herein refers a set of primers which can produce a double stranded nucleic acid product complementary to a portion of one or more RNA products of a gene of a SDPP gene set described in the application or sequences complementary thereof.

In one embodiment the primers are useful for quantitative multiplex PCR. Methods of designing primers suitable for multiplex PCR are known in the art. For example, SDPP gene specific primer pairs are first tested individually to find a PCR program that permits optimal amplification of all SDPP gene products and are then tested in combination to find a PCR program that is quantitative for all SDPP gene products being amplified.

In another aspect, the application provides probes that are useful for detecting the SDPP genes listed in Tables 3-6 and 9-11. In one embodiment, the probes include SEQ ID NOs: 13-16. The probe may optionally comprise parts of the aforementioned SEQ ID NOs which retain specificity for the target sequence recognized by the corresponding SEQ ID NO. For example the probe may comprise all of part of SEQ ID NO: 13, the part being sufficient to hybridize specifically to the nucleic acid or nucleic acids complementary to SEQ ID NO: 13.

Another aspect provides use of a collection of probes for detecting SDPP genes listed in Tables 3-6 and 9-11 and/or for detecting genes listed in Table 2. In one embodiment the nucleic acid sequences are the sequences as set out in Table 8. In another embodiment, the use comprises use of probes specific for one or more genes listed in Tables 3-6 and 9-11.

The term “probe” as used herein refers to a nucleic acid sequence that will hybridize to a nucleic acid target sequence. In one example, the probe hybridizes to an RNA product of a gene of a SDPP gene set described in the application or a nucleic acid sequence complementary to the RNA product of the a gene of a SDPP gene set described in the application. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

The probes in one embodiment are fixed to a solid support. In one embodiment the probes are fixed to an array chip such as a microarray chip. In a further embodiment, the microarray probes range from 25-70 nucleotides in length. In another embodiment the probes comprise cDNA and can be for example, 500-5000 nucleotides in length.

### Polypeptide Binding Compositions

The application describes a number of polypeptide products of SDPP genes and gene sets. In one aspect the application provides a composition comprising two or more SDPP polypeptides corresponding to SDPP genes. In one embodiment the composition comprises 3, 4, 5, 6, 7-10 or more polypeptides corresponding to SDPP genes. In another embodiment the composition comprises 11-14, 15, 16-18, 19, 20-25, 26, 27-29, 30-50, 50-100, 100-162, 163, 164-199 or 200 polypeptides corresponding to SDPP genes. In another embodiment the polypeptides correspond to genes selected from genes listed in Tables 3-5 and 9-11. In one embodiment the polypeptides correspond to genes selected from Table 2.

As mentioned above, the expression level of genes of a SDPP gene set can also be detected by detecting the expression of polypeptide products described in the application. Accordingly, another aspect of the application is a composition comprising a plurality of at least two binding agents, wherein each binding agent binds to a polypeptide product of a gene of a SDPP gene set, and wherein the composition is used to measure the level of expression of at least two genes of the SDPP gene set. The detected polypeptide gene products are selected from the genes presented in Tables 3-6 and 9-11. In one embodiment, at least 3, at least 4, at least 5, at least 6 or at least 10 polypeptide products of genes are detected. In a preferred embodiment, at least 3 polypeptide products of genes selected from Tables 9-11 are detected.

In one embodiment, the binding agent is an isolated polypeptide. The term “isolated polypeptides” as used herein refers to a proteinaceous agent, such as a peptide, polypeptide or protein, which is substantially free of cellular material or culture medium when produced recombinantly, or chemical precursors, or other chemicals, when chemically synthesized.

The phrase “bind to polypeptide products” as used herein refers to binding agents such as isolated polypeptides that specifically bind to polypeptide products of the SDPP genes described in the application. In an embodiment, isolated polypeptides are antibodies or antibody fragments.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

To produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with cancer cells and the monoclonal antibodies can be isolated.

Specific antibodies, or antibody fragments, reactive against particular SDPP gene polypeptide product antigens, may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

The application also contemplates the use of “peptide mimetics” for detecting the polypeptide products of SDPP genes. Peptide mimetics are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of the isolated proteins described in the application, such as its ability to bind to the polypeptide products of the SDPP genes described in the application. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to the cleavage recognition sequence described in the application.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

In one embodiment the binding agents are fixed to a solid support. In a further embodiment the solid support is an ELISA plate.

### Microarrays

As mentioned, the expression level of genes of a SDPP gene set is optionally detected using arrays including DNA microarrays and tissue microarrays. A “microarray: as used herein refers to a an ordered set of probes fixed to a solid surface that permits analysis such as gene analysis of a plurality of genes. A DNA microarray refers to an ordered set of DNA fragments fixed to the solid surface. For example, in one embodiment the microarray is a gene chip. A tissue microarray refers to an ordered set of tissue specimens fixed to a solid surface. For example, in one embodiment the tissue microarray comprises a slide comprising an array of arrayed tumor biopsy samples in paraffin. Tissue microarray technology optionally allows multiple specimens, such as biopsy samples, to be analysed in a single analysis at the DNA, RNA or protein level. Tissue microarrays are analysed by a number of techniques including immunohistochemistry, in situ hybridization, in situ PCR, RNA or DNA expression analysis and/or morphological and clinical characterization or a combination of techniques. The specimens are optionally from the same subject or from a plurality of subjects. Methods of detecting gene expression using arrays are well known in the art. Such methods are optionally automated. In one embodiment, a sample of a cancer patient is analysed using a tissue microarray. The sample is optionally used for clinical follow up to monitor the patient's progression.

Accordingly the application provides in one aspect an array comprising for each gene in a plurality of genes, the plurality of genes being at least 3 of the genes listed in Tables 3-6 or 9-11, one or more polynucleotide probes complementary and hybridizable to a coding sequence in the gene.

In one embodiment, the array comprises at least 15 genes listed in Table 9. In another embodiment the array comprises the genes listed in Table 9. In yet a further embodiment, the array comprises a substrate comprising a plurality of addresses, wherein each address has disposed thereon a capture probe that can specifically bind a gene of one or more SDPP gene sets of Tables 3-6 and/or 9-11.

In another aspect, the application describes methods for using an array described herein. In one embodiment, the application provides a method of predicting clinical outcome associated with a SDPP reference expression profile of a plurality of genes in a breast cancer patient comprising:

detecting the sample's gene expression levels using an array of described herein; comparing the gene expression levels to the SDPP reference expression profile of at least 3 genes of the SDPP gene set comprised on the array; and predicting clinical outcome associated the SDPP gene reference expression profile of the SDPP gene set;
wherein clinical outcome is predicted according to the probability of falling within the class defined the reference expression profile of the SDPP gene set.

In one embodiment, the microarray comprises one or more polynucleotide probes complementary and specific to one or more portions of a coding sequence for each gene of at least 3 genes listed in Tables 3-5 and 9-11. In one embodiment the microarray comprises polynucleotide probes complementary and specific to one or more portions of a coding sequence for each gene of at least 3 genes listed in Table 2.

### Methods of Diagnosis

The application discloses SDPP gene sets comprising genes which are differentially expressed in patients with different classes or subtypes of breast cancer. The subtypes are associated with different clinical outcomes or prognoses. Depending on the expression level of the SDPP genes in the patient sample, the breast cancer subtype is predicted to be associated with a good prognosis, a mixed prognosis or a poor prognosis. The subtypes are differentially associated with recurrence and metastasis. Accordingly, one aspect described in the application is a method of diagnosing a breast cancer subtype in a breast cancer patient. In another embodiment the application provides a method of providing a prognosis. In one embodiment, the application provides a method of predicting or diagnosing recurrence. In another embodiment the application provides a method of predicting metastasis.

Clinical outcome is predicted by methods comprising the comparison of expression level of at least 3 genes or at least 5 genes of a SDPP gene set selected from Tables 3-6 and 9-11 in a sample of a patient to the reference expression profile of the corresponding genes derived from tumor associated stroma and predicting clinical outcome on the statistical probability of falling within the class defined by the reference expression profile of the at least 3 or at least 5 genes. In one embodiment the SDPP gene set comprises a gene set provided in Tables 9-11. In another embodiment, the SDPP gene set is the gene set provided in Table 9.

Prognosis is predicted by methods comprising the comparison of expression level of at least 3 genes of a SDPP gene set selected from Tables 3-6 and 9-11 in a sample of a patient to the reference expression profile of the corresponding genes derived from tumor associated stroma and providing prognosis on the statistical probability of falling within the class defined by the reference expression profile of the at least 3 genes. In one embodiment at least 5 genes of a SDPP gene set selected from Tables 3-6 and 9-11 in a sample of a patient to the reference expression profile of the corresponding genes derived from tumor associated stroma and providing prognosis on the statistical probability of falling within the class defined by the reference expression profile of the at least 5 genes. In one embodiment the SDPP gene set comprises a gene set provided in Tables 9-11. In another embodiment, the SDPP gene set is the gene set provided in Table 9.

Recurrence is predicted by methods comprising the comparison of expression level of at least 3 genes of a SDPP gene set selected from Tables 3-6 and 9-11 in a sample of a patient to the reference expression profile of the corresponding genes derived from tumor associated stroma and predicting the likelihood of recurrence on the statistical probability of falling within the class defined by the reference expression profile of the at least 3 genes. In one embodiment, the method comprises the comparison of at least 5 genes. In one embodiment the SDPP gene set comprises a gene set provided in Tables 9-11. In another embodiment, the SDPP gene set is the gene set provided in Table 9.

Metastasis is predicted by methods comprising the comparison of expression level of at least 3 genes of a SDPP gene set selected from Tables 3-6 and 9-11 in a sample of a patient to the reference expression profile of the corresponding genes derived from tumor associated stroma and predicting the likelihood of metastasis on the statistical probability of falling within the class defined by the reference expression profile of the at least 3 genes. In one embodiment, the method comprises the comparison of at least 5 genes. In one embodiment the SDPP gene set comprises a gene set provided in Tables 9-11. In another embodiment, the SDPP gene set is the gene set provided in Table 9.

The term “patient” also referred to as “subject” as used herein refers to any member of the animal kingdom, preferably a human being.

The term “diagnosis” as used herein refers to identifying the nature of the disease or identifying the cause or outcome of a disease or group of related diseases such as breast cancer.

In certain embodiments the expression level of at least 3 genes or at least 5 genes of a SDPP gene set is obtained by detecting the expression level of the genes in a patient sample. A person skilled in the art will appreciate that a number of methods can be used to measure or detect the level of RNA products or complementary DNA of a gene of a SDPP gene set described in the application within a sample, including microarrays, RT-PCR (including quantitative RT-PCR and multiplex quantitative RT-PCR), nuclease protection assays and northern blots. In a preferred embodiment detection comprises a quantitative multiplex PCR method. In another embodiment detection comprises a microarray method.

In addition to measuring the expression of RNA products of genes of SDPP gene sets described in the application, differential expression of the polypeptide products of the SDPP genes described in the application can be used to predict disease outcome or diagnose cancer subtype. Accordingly, another aspect of the application is a method of predicting disease outcome or diagnosing cancer subtype comprising detecting the level of a plurality of at least two polypeptide gene products, each polypeptide gene product corresponding to a gene in a SDPP gene set.

In one embodiment of the application antibodies or antibody fragments are used to determine the level of polypeptide product of one or more genes of a SDPP gene set described in the application. In one embodiment the isolated polypeptides are labeled with a detectable marker.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a secondary antibody that is specific for the isolated protein described in the application and contains a detectable label can be used to detect the isolated polypeptide described in the application.

A person skilled in the art will appreciate that a number of methods can be used to determine the amount of the protein product of a gene of a SDPP gene set described in the application, including immunoassays such as Western blots, ELISA, and immunoprecipitation followed by SDS-PAGE, as well as immunocytochemistry or immunohistochemistry.

In one embodiment at least 1, 2, 3, 4, 5 or more than 5 polypeptide gene products of a SDPP gene set are detected by detecting the polypeptide level of the corresponding gene.

In addition detection of a level of gene expression of more than one gene of a SDPP gene set is in one embodiment, accomplished by combining detecting nucleic acid and polypeptide gene product expression levels. For example in one embodiment, the levels of gene expression of 5 genes of a SDPP gene set are obtained by detecting polypeptides of one or more genes of the SDPP gene set, and by detecting RNA expression of one more genes of the SDPP gene set such that a total of 5 gene expression levels are detected. In addition any of the methods described herein are optionally used in addition or in combination with traditional diagnostic techniques for breast cancer.

Integration with Other Gene Sets or Prognostic Factors

A number of other predictors have been identified including the 70-gene predictor, the wound signature and the hypoxia signature³′¹⁹′²°.

The inventors have further shown that the accuracy of predicting disease outcome is enhanced when combined with other predictors such as those described above. For example the inventors have demonstrated that combining the SDPP with a number of predictors including the 70-gene predictor, the wound response and hypoxia signatures, increases the accuracy in predicting metastasis and good outcome. Accordingly, one aspect of the application provides a method integrating a method of predicting disease outcome using at least 3 genes of a SDPP gene set with other predictors. In one embodiment, the SDPP is combined with other predictors for predicting likelihood of metastasis.

### Methods of Assigning or Selecting Treatment

The inventors have found that the SDPP is able to stratify patients according to clinical outcome with a greater degree of accuracy than other known predictors. This allows the opportunity for clinicians to tailor treatment and reserve more aggressive therapies with greater risk or side effects for patients with poorer outcome.

Accordingly, one aspect described in the application provides assigning treatment to a patient according to the predicted clinical outcome of the patient. Assigning treatment can be challenging for breast cancer subtypes that are associated with good prognostic factors such as ER positive, HER2 negative or low/no lymph node involvement breast cancers. A subset of these patients show poor outcome. The reverse is also true. A subset of cancer subtypes associated with poor prognostic factors show good outcome. Accordingly, in one embodiment, the patient has a HER2 positive breast cancer with good outcome. In another embodiment, the patient has a HER2 positive breast cancer with poor outcome. In another embodiment, the patient has a HER2 negative breast cancer with good outcome. In another embodiment the patient has a HER2 negative breast cancer with poor outcome. In another embodiment the patient has an ER positive breast cancer. In yet a further embodiment, the patient has an ER negative breast cancer.

Another aspect relates to monitoring treatment efficacy. Gene expression of at least 3 genes of a SDPP gene set is assessed and reassessed at a subsequent time point after initiation of a treatment. A change in the expression levels from one class of clinical outcome, wherein the change is from a poor to a mixed or good clinical outcome, is indicative of treatment efficacy. Similarly a change from a mixed clinical outcome to a good clinical outcome is indicative of an efficacious treatment regimen. On the other hand a change from a good to mixed or poor clinical outcome suggests treatment failure.

Accordingly, the application provides in one embodiment a method of monitoring effectiveness of a treatment in a breast cancer patient comprising:
a) obtaining an expression level for at least 3 genes of an SDPP gene set in a first sample of a patient, wherein the first sample is taken before or after the start of the treatment;
b) obtaining an expression level for at least 3 genes of a SDPP gene set in a second sample of a patient, wherein the second sample is taken subsequent to the first sample and after at least one treatment;
c) comparing the expression levels of the genes in the first and second sample to the reference expression profile of the genes in the SDPP gene set; and
d) determining the disease outcome class for the first and second sample;
   wherein a change in the outcome class of sample 2 indicating a decreased probability of poor prognosis indicates the treatment is effective.

Analysis of the SDPP gene sets has also revealed several gene clusters that are associated with clinical outcome. For example, the inventors have shown that the tumor associated stroma of patients with poor outcome is enriched for genes involved in a Th2 immune response, hypoxia and angiogenesis. These genes include adrenomedulin, interleukin 8, CXCL1, MMP12 and MMP1. Stromal changes during breast cancer progression may include the induction of hypoxia, which promotes recruitment of immune cells and endothelial cells, providing growth and matrix remodeling factors as well as a new blood supply for the tumor. Local activation of fibroblasts enhances matrix remodeling, facilitating tumor cell invasion. Normally, the interplay between epithelial cells and the microenvironment maintains epithelial polarity and modulates growth inhibition¹⁴. Modification or destabilization of the microenvironment can lead to loss of epithelial cell polarity and increased cell proliferation, contributing to tumorigenesis^{14,21,22} Other tumor cell-microenvironment interactions can allow the tumor to escape immune surveillance and promote tumor growth and metastasis¹⁷.

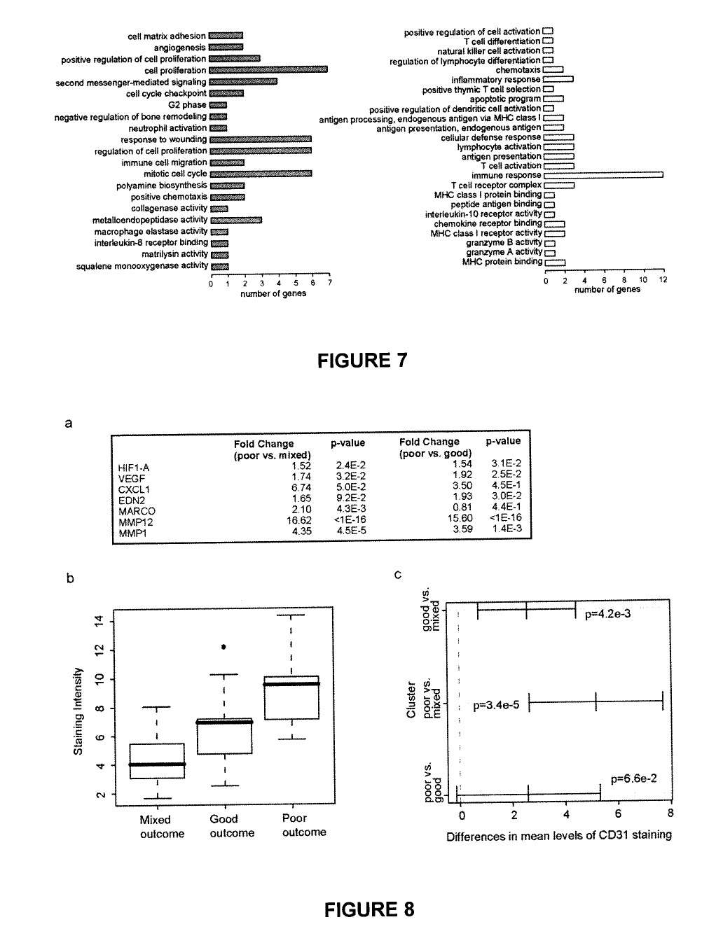

The inventors have further shown that genes expressed in the good outcome patient cluster are enriched for gene involved in the Th1 type immune response, including T cell selection and differentiation, MHC class 1 receptor activity and granzyme NB activity (FIG. 7) implying incrased recruitment of activated T-cells and NK cells in these tumors.

Accordingly the application provides methods of treatment according to the transcriptional profile of tumor associated stroma and/or the clinical class predicted. In one embodiment patients predicted to have a poor clinical outcome are assigned therapies that target Th2 immune responses, angiogenesis processes and/or hypoxic processes. In one embodiment, the application provides a method of optimizing treatment. In another embodiment, the treatment regimen includes a component that promotes a Th1 immune response. In another embodiment the treatment regimen includes a component that inhibits a Th2 immune response. A treatment regimen is chosen that is tailored to the biological responses activated in the patient.

### Novel Therapeutics

The application also provides in one aspect a method of identifying agents for use in the treatment of cancer. Clinical trials seek to test the efficacy of new therapeutics. The efficacy is often only determinable after many months of treatment. The methods disclosed herein are useful for monitoring the expression of SDPP genes associated with recurrence, metastasis or poor prognosis. A change in SDPP gene expression levels which are associated with a better prognosis are indicative of treatment efficacy.

Accordingly in one embodiment, the application provides a method for identifying agents for use in treatment of breast cancer comprising:
a) obtaining an expression level for at least 3 genes of an SDPP gene set in a first sample of a cell culture;
b) incubating the cell culture with a test agent;
c) obtaining an expression level for the at least 3 genes in a second sample, wherein the second sample is subsequent to incubating the cell culture with the test agent;
d) comparing the expression level of the at least 3 genes in the first and second sample to a reference expression profile of the genes;
   wherein a change in the expression level of the genes in the second sample indicating a decreased probability of falling within a poor prognosis class indicates that the agent is useful for the treatment of breast cancer.

A person skilled in the art will be familiar with various cell culture techniques and cell lines that are useful for the methods described herein.

Further, the inventors have disclosed that specific pathways are activated in different classes of clinical outcome. The application provides in one embodiment a method to identify and test the efficacy of treatments targeted to these deregulated pathways. In one embodiment the method comprises identifying an agent that inhibits expression of hypoxia response genes implicated in poor prognosis. In another embodiment, the method comprises identifying an agent that inhibits expression of Th2 response genes associated with poor prognosis. In a further embodiment, the method comprises identifying an agent that inhibits expression of angiogenesis genes associated with poor prognosis.

### Kits

Another aspect of the application is a kit for predicting disease outcome in a patient, classifying tumor subtype, monitoring treatment and disease progression and for diagnosing or detecting cancer comprising any one of the isolated nucleic acid compositions described in the application and instructions for use. In a preferred embodiment the kit comprises nucleic acid compositions for carrying out multiplex PCR.

In one embodiment the application provides a kit for classifying a breast cancer comprising:
a plurality of isolated nucleic acids for detecting expression levels of at least 3 genes of a SDPP gene set; and instructions for use.

In another embodiment the kit the isolated comprises nucleic acids that are primers useful for amplifying the expression products of the at least 3 genes. In another embodiment the kit the primers comprise one or more of the primers selected from the group consisting of SEQ ID NO: 1-12. In yet another embodiment, the kit comprises isolated nucleic acids wherein the nucleic acids are probes that hybridize expression products of the at least 3 genes.

In one embodiment, the invention provides a kit comprising an array chip such as a microarray chip for predicting disease outcome in a patient, classifying tumor subtype, monitoring treatment and disease progression and for diagnosing or detecting cancer.

A further aspect is a kit for predicting disease outcome in a patient, classifying tumor subtype, monitoring treatment and disease progression and for diagnosing or detecting cancer comprising any one of the isolated polypeptides described herein and instructions for use. In one embodiment, the isolated protein is labeled using a detectable marker.

### Computer Systems

The application also provides for a computer system for use with the methods described in the application. In another embodiment the application provides for a computer program product for implementing the methods described in the application. In a further embodiment, the application provides a computer readable medium having stored thereon a data structure for storing a method described in the application.

Accordingly the application provides a computer system comprising:
a) a database including records comprising the reference expression profiles of a plurality of genes in Tables 3-6 and/or 9-11;
b) a user interface capable of receiving a selection of gene expression levels of at least 3 genes in Tables 3-6 and/or 9-11 for use in comparing to the tumor associated gene reference expression profiles in the database;
c) an output that displays a prediction of clinical outcome according to the expression levels of the at least 3 genes.

In another embodiment the application provides a computer readable medium on which is stored a database capable of configuring a computer to respond to queries based on records belonging to the database, each of the records comprising:
a) a value that identifies a gene of a SDPP gene set;
b) a value that identifies the probability of a clinical outcome associated with the gene.

The computer readable medium on which is stored a database capable of configuring a computer to respond to queries based on records belonging to the database, each of the records comprising:
a) a value that identifies a gene reference expression profile of a SDPP gene set;
b) a value that identifies the probability of a clinical outcome associated with the gene reference expression profile.

In yet another embodiment the application provides a computer readable medium comprising a plurality of digitally encoded reference expression profiles, wherein each profile of the plurality has a plurality of values, each value representing the expression of a different gene of a SDPP gene set. In one embodiment the computer readable medium includes program instructions for performing the following steps:
a) comparing a plurality of gene expression levels of a patient sample with a database including records comprising the reference expression profiles of a plurality of genes in Table 2-6 and/or 9-11 and associated clinical outcome weighting to predict the clinical outcome of the patient; and
b) providing the clinical outcome prediction with the identified gene expression levels.

The following non-limiting examples are illustrative of the present invention:

### EXAMPLES

### Example 1

### Methods

### Description of Samples

Tissue samples from 73 patients presenting with invasive ductal carcinoma (IDC) were subjected to laser capture microdissection (LCM). From this cohort, 53 samples were obtained of tumor-associated stroma; in 31 cases, patient-matched normal adjacent stroma was also obtained. The median follow-up of our patients was 3.44 years. Recurrence (local or distant) was determined by examination of medical records following diagnosis. Poor outcome was defined as alive with disease or dead of disease as of the time of the latest follow-up. No patient in the study received neoadjuvant therapy. This study was approved by the McGill University Health Centre (MUHC) Research Ethics Board (protocols SUR-00-966 and SUR-99-780), and all subjects provided written, informed consent.

### LCM, RNA Isolation and Microarray Hybridization

Regions of tumor-associated and normal stroma were identified by a clinical pathologist prior to microdissection. LCM, sample isolation and preparations, as well as microarray hybridization, were carried out as previously described²³. Normal stroma was harvested at least 2 mm away from the tumor margins. Each RNA sample was hybridized on Agilent 44K whole human genome microarrays in a dye-swap replication design; 50 samples were hybridized in duplicate, one in triplicate, and two in quadruplicate. In total, 459 arrays were obtained. After performing normalization and model fitting as previously described^{23,24}, our microarray dataset contained 111 distinct expression experiments.

Identification of a Tumor Stroma Subtype Associated with Recurrence and Poor Outcome

A LIMMA²⁵ model was fit to the patient-matched tumor-associated vs. normal stroma data, and identified the top 200 most variable genes across all patients, which were also differentially expressed in at least 3 patients (p<1e-5). The 200 genes chosen were in the 99.2% percentile of the variance distribution. This approach excluded genes that co-vary between tumor associated and normal stroma. Tumor associated stroma was clustered using these genes and the significance of clusters was assessed by bootstrapping (1000 bootstrap iterations) using the pvclust package²⁶. Each cluster was tested for association with ER, PR, lymph node, HER2 and p53 status, as well as grade, recurrence, and outcome, using a CHI² association test

### Identification of Genes Differentially Expressed Between the Tumor Associated Stroma Subtypes

Pair-wise class distinction was used to identify genes differentially expressed between the poor outcome, mixed outcome, and good outcome associated stroma subtypes previously defined by class discovery. The expression profile of the outcome-associated tumor stroma subtypes was derived from the union of differentially expressed genes identified using SAM²⁷ (multiclass comparison, q-value<0.01), and LIMMA (intersection of top 200 differentially expressed for each comparison, ranked by fold change FDR adjusted p-value<0.01) algorithms for differential expression.

### Predictor Construction and Evaluation

Logistic regression was used to score and rank each gene in the expression profile, based on its significance in estimating binomial recurrence in a model including gene expression level, lymph node status, estrogen receptor status, progesterone receptor status and HER2 receptor status. This model ensured that the predictive strength of a gene was not confounded with lymph node, ER, PR, or HER2 status⁴.

Naïve Bayes' classifiers were trained to predict prognosis using the ranked gene expression profile of the recurrence-positive stroma cluster. Each classifier was trained on an incrementally larger set of genes from the ranked list, and then evaluated using 50 cross validation runs by randomly splitting the data into testing and training sets of equal size (n=27 training samples, n=26 testing samples). Receiver-operator-characteristic (ROC) curves were generated for each classifier, and classifiers were compared using their area under the curve (AUC). The optimal predictor was selected to maximize the AUC, and trained on all the data (n=53 samples). The performance of the SDPP in tumor associated stroma was compared to its performance in tumor epithelium, normal stroma, and normal epithelium using the AUC.

### Gene Ontology (GO) Analysis

Genes differentially expressed in each stroma subtype were cross-referenced against Gene Ontology (GO) annotations²⁸ to identify overrepresented GO categories using a test against the hypergeometric distribution, using a significance threshold of p≦0.05.

Comparison with Publicly Available Breast Cancer Datasets

Publicly available breast cancer data from four different studies^{4,12,18,29} was downloaded and the SDPP was used to predict the outcome for each patient. In the NKI and Wang et al. data sets^{12,18}, the poor, good, and mixed-outcome categories of samples identified by the SDPP were treated as categorical variables in Cox proportional hazards regression. These included age, HER2 status, ER status, grade, lymph node status, as well as predictions from the 70-gene predictor, and wound, and hypoxia signatures as other clinical risk factors. Tests were performed for association with both overall survival and recurrence-free survival.

### Expression of Macrophage, Angiogenesis, Hypoxia and Immune Markers

ANOVA and Tukey's Honest Significant Difference test (HSD) were used to evaluate differences in the level of expression of selected macrophage, angiogenesis, immune, and, hypoxia-related markers between the three clusters of outcome-associated stroma identified in FIG. 2a. The genes analyzed were HIF1A, CXCL1, EDN2, MSR1, MARCO, MMP1, MMP12, and CCL2.

### Functional Annotation of Unknown Predictor Genes

Gene symbols in the list of 163 differentially expressed genes were obtained from the BioConductor annotations for the Hgug4112a Agilent array. Symbols beginning with THC reference The Institute for Genomic Research (TIGR) Tentative Human Consensus (THC) sequences. Unknown probes were blasted against the ENSEMBL human genome assembly (release 45). The SDPP member gene THC2394165 was found to have a probe that aligned immediately upstream of SNTG2 (gamma-2 syntrophin). Correlation between the probes for STNG2 and THC2394165 was 0.42. This is in the 99^{th} percentile of correlations between these probes and all other probes on the array, strongly suggesting that the probe for THC2394165 is detecting expression of SNTG2.

### Immunohistochemistry

Expression of proteins corresponding to selected members of the SDPP gene set (CD8, CD3z and osteopontin/SPP1) was validated by immunohistochemistry, using sections from formalin-fixed paraffin-embedded blocks obtained from the MUHC Pathology archive, while CD31 expression was evaluated on frozen tissue sections. Procedures were carried out as per the manufacturer's instructions (see Table 7 for details). Slides were then scanned using an Aperio ScanScope XT (Aperio Technologies, Vista, Calif.) with a 20× objective and images extracted using the ImageScope image viewer (Aperio Technologies).

### Q-RT-PCR

Amplified RNA (aRNA) prepared from microdissected tissues were used as a templates for RT-Qt PCR validation using a LightCycler instrument (Roche Applied Science) as per the manufacturer's instructions. Briefly, reactions for CXCL1, VGLL1 and LCP1 were performed using the appropriate Universal Probe Library (Roche) probes, while reactions for ADM, CD8A and SPP1 were performed using probes designed using the OligoPerfect™ Designer software (Invitrogen). aRNA was initially reverse transcribed using AMV reverse transcriptase (Roche). All primers and probe sequences were designed within 300 by of the 3′-end. Primer sequences and Universal Probe Library probes are described in Table 8. The crossing point was automatically calculated using the LightCycler 3.5 software and determined from the second derivative maximum on the PCR amplification curve. Transcript quantification was performed by comparison with standard curves generated from dilution series of cDNA from pooled connective aRNA (crossing point vs. log initial RNA amount). Melt curve analyses confirmed that single products were amplified. Agarose gel electrophoresis was used to establish that PCR products were of the predicted length.

### Results

Gene Expression in Breast Tumor Stroma Identifies Clusters Associated with Outcome

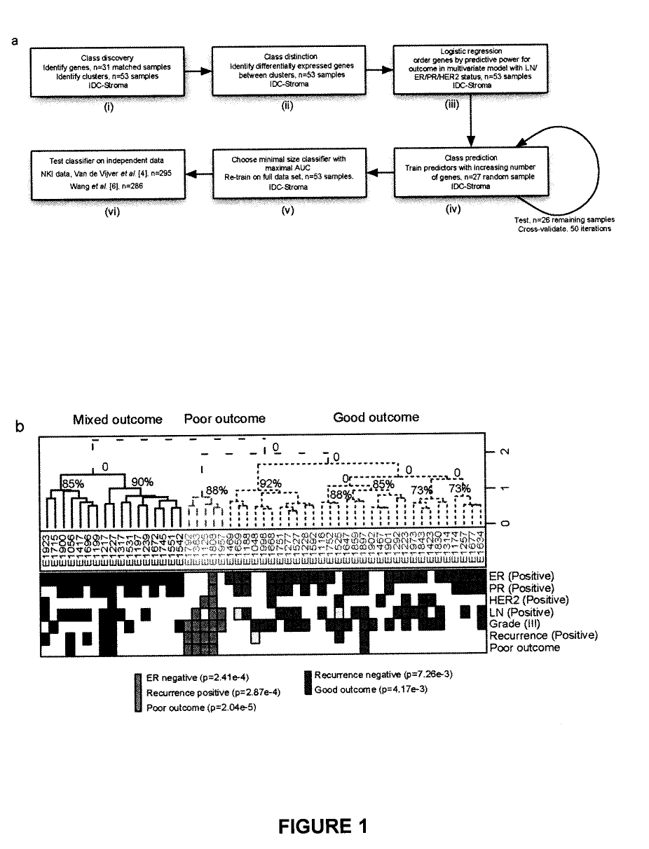

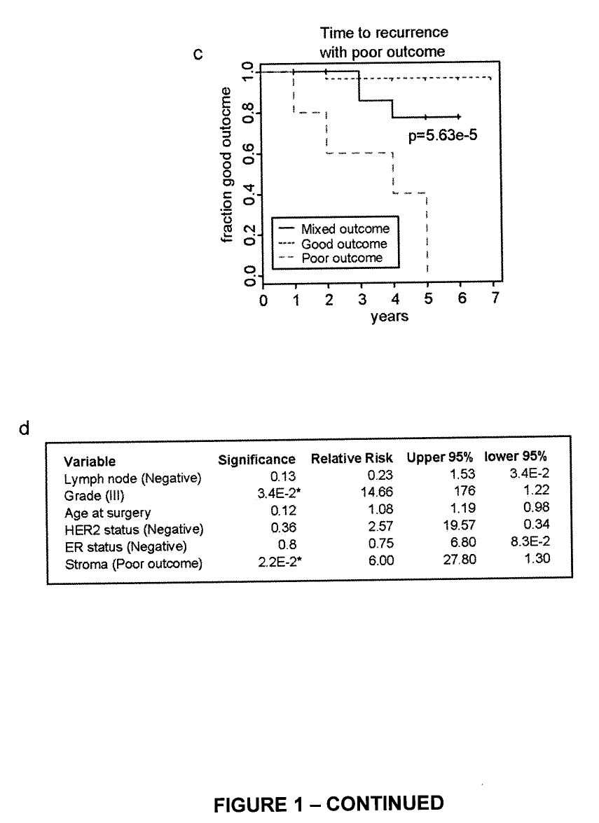

To investigate changes in breast tumor-associated stroma LCM-based tissue isolation and RNA amplification were combined with gene expression profiling using DNA microarrays²³. LCM was used to collect cells from the stromal compartment within the tumor bed and within adjacent normal tissue from 53 patients presenting with invasive ductal carcinoma (IDC) (Table 1). From 31 of these patients, data was obtained for matched tumor-associated and normal stroma. In order to determine whether gene expression in tumor-associated stroma could identify patient subtypes as has previously been observed in analysis using whole tissue⁴, a class discovery approach was applied. Therefore, a list of genes whose expression showed the most variation between the matched tumor versus normal stroma expression was generated for the 31 tissue-matched patients. The 200 most variable genes (Table 2) were used to cluster the complete data set of 53 patient tumor stroma samples (FIG. 1a-i). This class discovery analysis identified three patient clusters (FIG. 1b). One cluster (good outcome, FIG. 1b, 1c) has a significantly reduced rate of recurrence and longer relapse-free survival (RFS) (p=7.26e-3 and p=4.17e-3, respectively, c² test for association), while a second patient cluster (poor outcome, FIG. 1b, 1c) has a significantly increased rate of recurrence and shorter RFS (p=2.04e-5 and p=2.87e-4, respectively). The third (FIG. 1b, lc) contains patients with mixed outcomes. Unlike similar analyses using breast cancer datasets derived from whole tissue where patients cluster predominantly based on ER and HER2 status³⁰, multivariate Cox regression indicates that the poor outcome patient cluster identified by stromal gene expression is independent of ER, HER2 and lymph node status, as well as age and grade, (FIG. 1d). Hence the stroma-derived patient clusters are distinct from previously identified breast tumor subtypes⁴.

### Good and Poor Outcome Patient Stroma Exhibits Distinct Biological Responses

The tri-partition of the patients by stromal expression profiles may represent three subtypes of breast tumor-associated stroma (FIG. 1b). To investigate if the differences between these patient groups reflect distinct biological responses that can be used to distinguish between the patient subgroups, genes differentially expressed between each patient cluster were identified. Using the complete unmatched tumor stroma gene expression data from the 53 patients, pairwise comparisons of gene expression between the three patient clusters were performed (FIG. 1a-ii). From this class distinction, 163 distinct genes were identified that have the greatest differences in expression between clusters (FIG. 2, Tables 3, 4, 5). Using this gene set, patients cluster by outcome in a manner similar to that previously generated by class discovery (FIG. 2a, b). The 163-gene set was then used as a starting point to characterize the differences between the good and poor outcome-associated stroma subtypes at the molecular level. These 163 genes cluster into three distinct groups (FIG. 2a, gene clusters identified as 1, 2 and 3).

Each stroma patient subtype (FIG. 2a, good, poor and mixed-outcome patient clusters) contains several genes whose expression is elevated in that subtype and which are involved in distinct biological responses, providing evidence that each stromal subtype reflects different biologies. For example, gene cluster 2 (FIG. 2c) contains 102 genes specifically elevated in the poor-outcome patient cluster. Gene Ontology (GO) analysis of these genes (FIG. 7) identifies an enrichment for functions and processes previously associated with poor outcome³¹′³². These genes include factors associated with an angiogenic response, such as adrenomedullin (ADM), interleukin 8 (IL8) and CXCL1³³⁻³⁵. Supporting the link to angiogenesis, patients within our poor outcome cluster exhibit the highest levels of endothelial content, as established by immunostaining with the endothelial marker CD31 (FIG. 8 b, c, d). Several matrix metalloproteinase genes are highly expressed in poor vs good outcome, (MMP12 and MMP1 respectively, poor vs good 15.6 and 3.59-fold differential expression, respectively, pvalues<1e-1 and 0.0014, respectively). MMP1 and MMP12 are known factors involved in tissue remodeling by macrophages. MMP1 is also linked to angiogenesis, invasion and metastasis³⁶. Additionally, adrenomedullin has been previously identified as part of a hypoxia transcriptional response¹⁹.

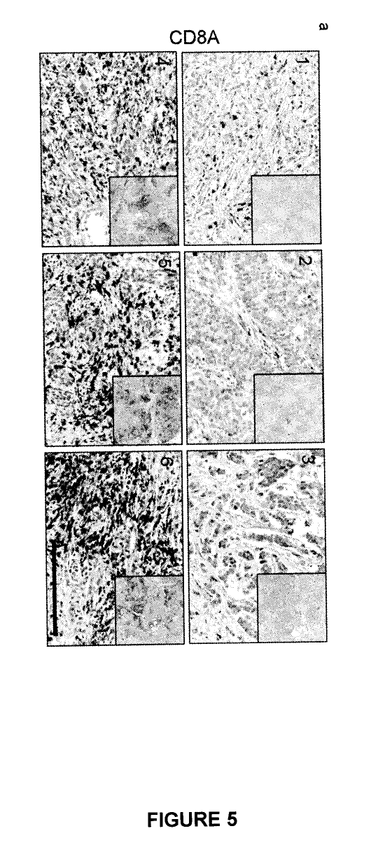

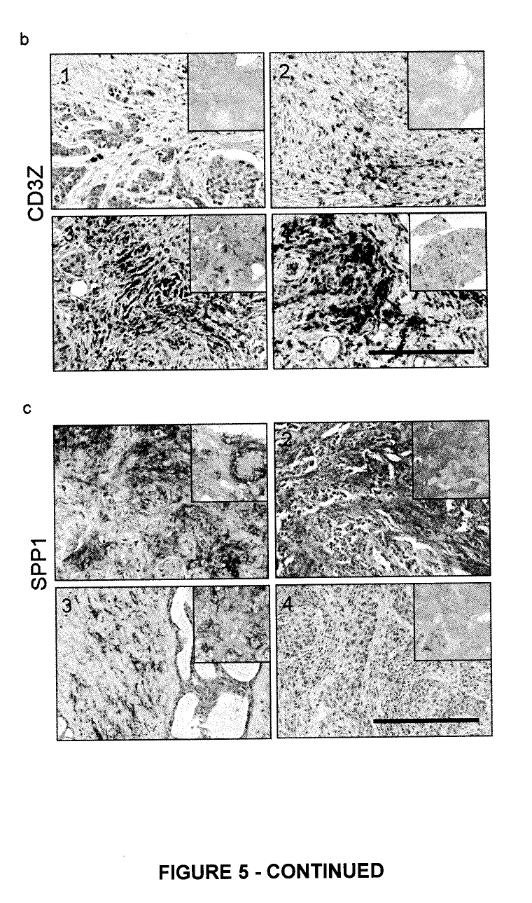

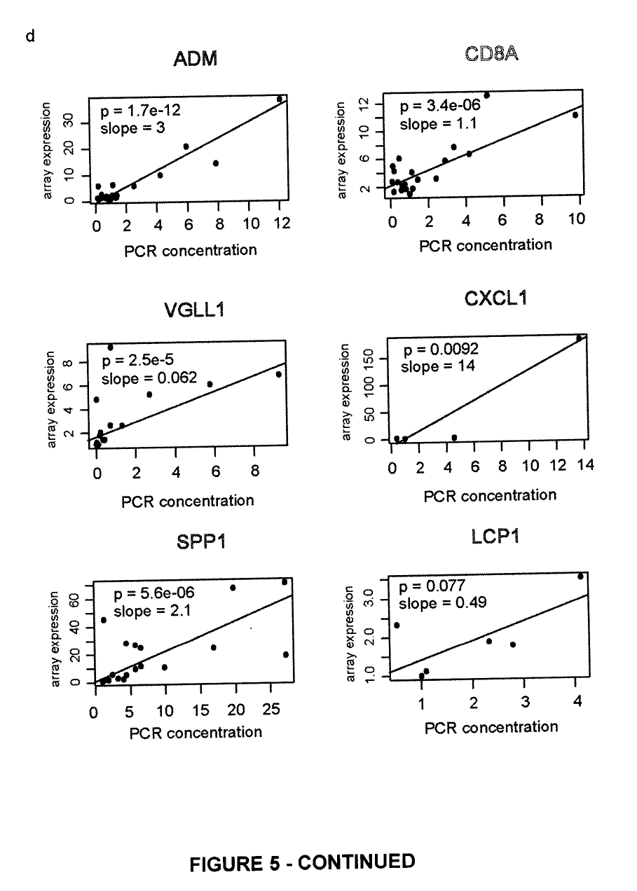

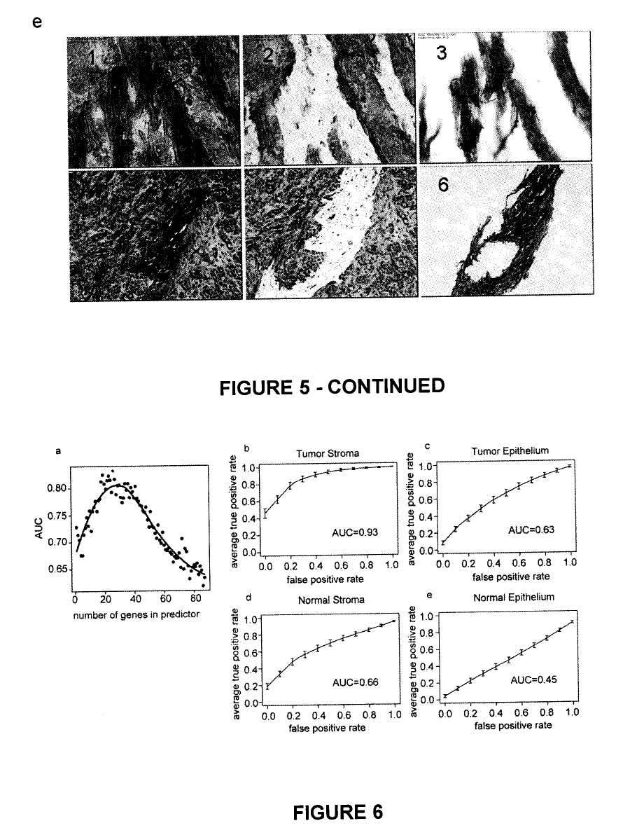

There are 29 genes predominantly expressed in the good outcome patient cluster (FIG. 2a, cluster 2 2e). GO analysis demonstrates enrichment for genes involved in the Th1-type immune response, including T-cell selection and differentiation, MHC class I receptor activity, and granzyme NB activity (FIG. 7). This implies that increased recruitment of activated T-cells and NK (natural killer) cells occurs in these tumors (FIG. 2a, good outcome cluster). Using immunohistochemistry it was confirmed that elevated levels of CD8 and CD3Z-positive cells are present in sections of tumor-associated stroma from patients in the good versus poor outcome-linked clusters (FIG. 5 a, b).

There are 33 genes expressed in samples from both good and mixed-outcome patient clusters (FIG. 2a cluster 3 2d). GO analysis identifies enrichment for estrogen and androgen receptor activity and positive regulation of cell proliferation, among others, consistent with the preponderance of ER-positive patients in this cluster.

### Construction of a Stroma-Derived Prognostic Predictor

Based on the 163-gene signature of tumor-associated stroma subtypes, a minimal subset of these genes was identified that can act as a predictor of outcome. Many factors known to have prognostic value for breast cancer outcome, such as ER or HER2 status, can significantly affect tumor gene expression profiles⁴. To limit the influence of these effects, genes predictive of outcome independent of these factors were identified. Multivariate logistic regression, with ER, PR, HER2 and lymph node status as covariates, was used to rank genes from most to least significant by their independent prognostic ability (FIG. 1a, iii, see Materials and Methods). Thus genes at the top of this list (Table 3) are more likely to be independent predictors of outcome. To construct a multivariate predictor of outcome, a multivariate naïve Bayes classifier was trained using incrementally larger gene sets from the ordered list (Table 3, FIG. 1a-iv). Each classifier was evaluated using 50 cross-validation runs, randomly splitting the data into testing and training sets. Receiver-operator characteristic (ROC) curves and the area under the curve (AUC) were used to assess the classifiers. Although there were a number of predictors with similar performance (FIG. 6 a), the predictor that maximized the AUC contained 26 genes (FIG. 1a-v) and performed well only in tumor-associated stroma (FIG. 6 c, d, e). [Notably, these genes contain representatives from each of the three gene clusters (gene clusters 1, 2 and 3) identified from the 163-gene set (FIG. 2a). Expression of selected genes within the predictor was validated by quantitative real-time PCR and significant correlations were found with array data (FIG. 5 d). Attempts to identify highly accurate predictors using other, more parsimonious, approaches to this problem failed. For example, predictors learnt directly from the list of differentially expressed genes between good and poor outcome patients had significantly less predictive ability than the 26-gene set learned from the 163-gene stroma signature.

Performance of the Stroma-Derived Prognostic Predictor (SDPP) in Datasets Derived from Whole Tissue

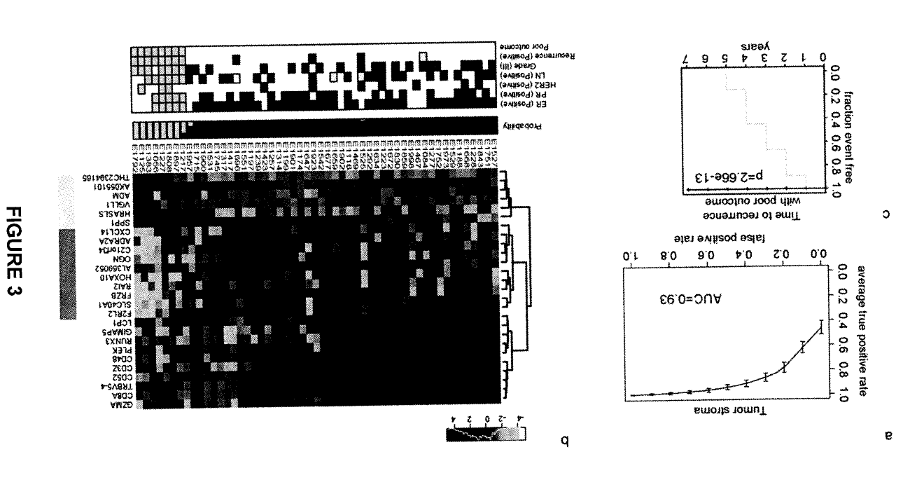

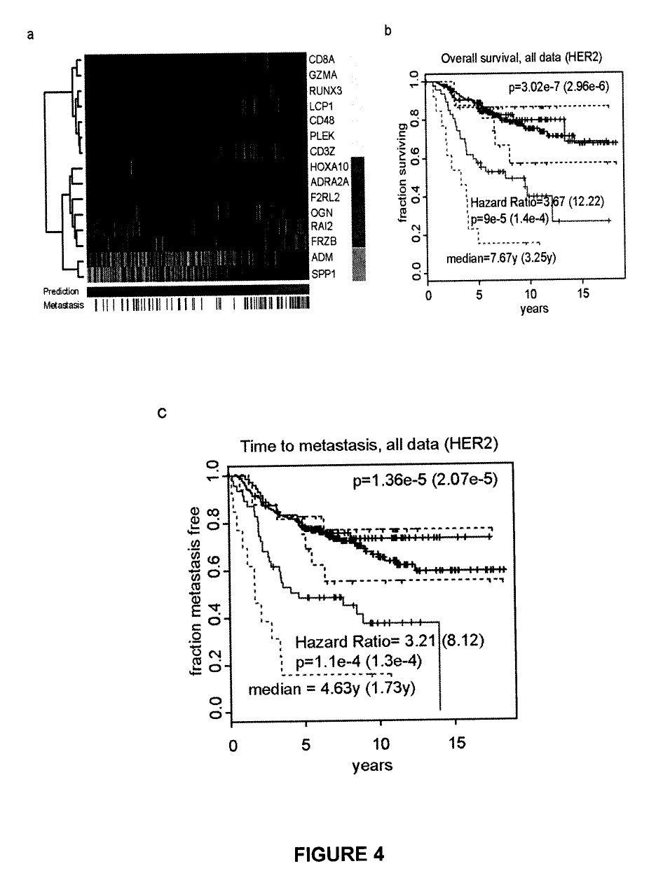

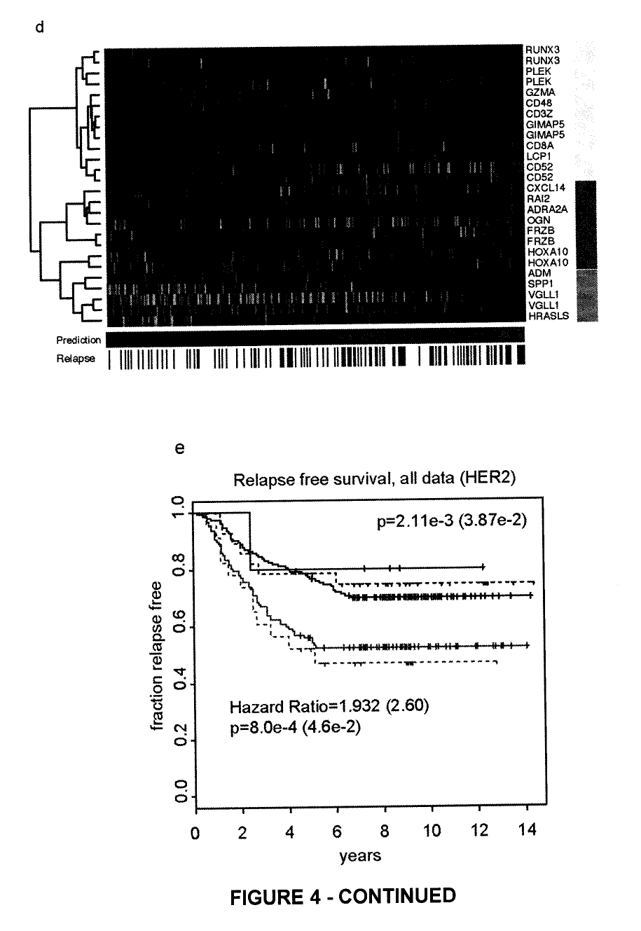

Previous analyses have derived predictors for outcome from data derived from whole breast tumor tissue, containing tumor and stroma^{3,12}. To establish whether our SDPP could successfully predict outcome in such data, several breast cancer datasets were examined. Two large publicly available examples have been analyzed extensively (van de Vijver et al.¹⁸ (NKI) and Wang et al.¹²) (FIG. 1a-vi). The NKI dataset consists of 295 IDC breast cancer samples with mixed ER, PR, HER2, and lymph node status, while the Wang et al. dataset contains 286 lymph node-negative cases. Only a subset of genes from the SDPP predictor were present on the arrays used for each of these datasets (15/26 for NKI and 19/26 for Wang et al.). Using these genes, the outcome of each sample using the SDPP classifier was predicted (FIG. 4a, d respectively; good, mixed, poor). In both datasets, patients assigned to the poor-outcome group by our SDPP are at significantly increased risk of recurrence and death from disease when compared to patients in the other two groups (FIG. 4 b,c.e) demonstrating the utility and robustness of the predictor in data derived from whole tissue. Moreover, since all patients in the Wang et al.¹² dataset were node-negative, our analysis demonstrates that gene expression in tumor-associated stroma is predictive of outcome prior to node involvement.

### The SDPP is an Independent Prognostic Factor

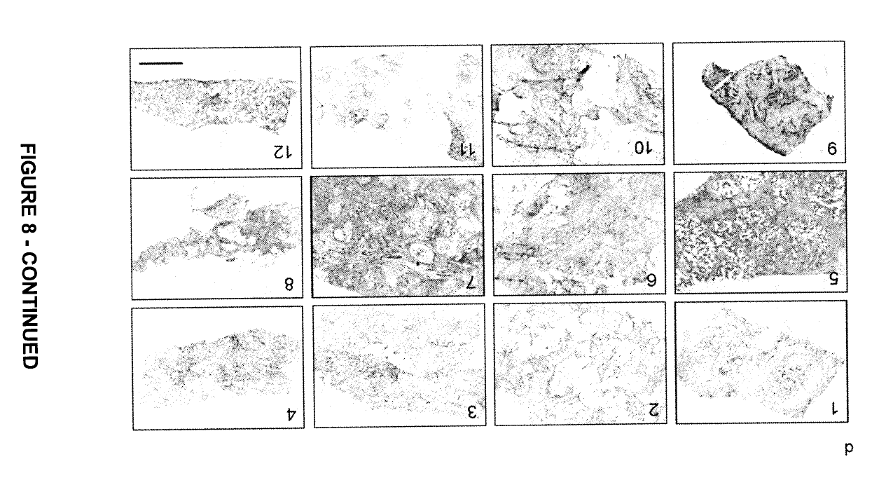

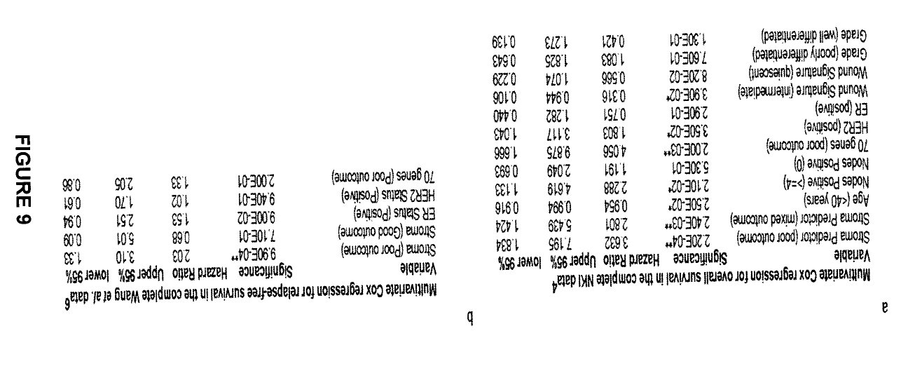

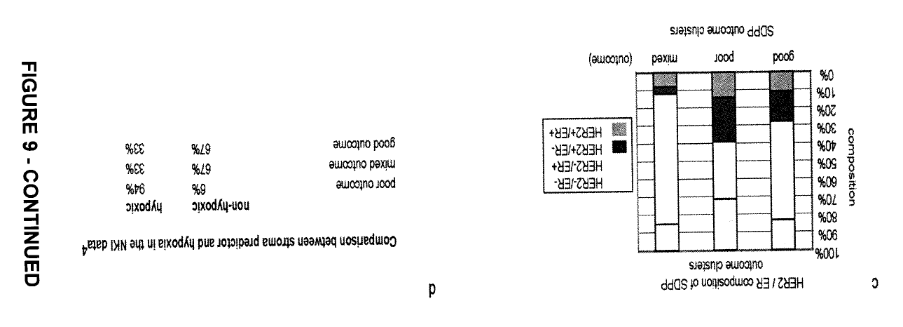

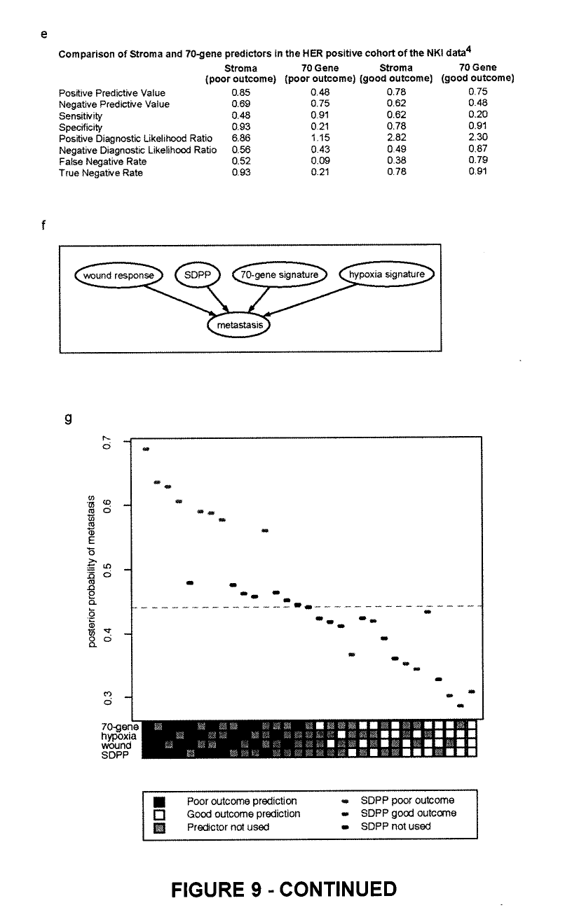

To test whether the SDPP was an independent prognostic factor, the composition of the SDPP patient clusters was examined, and multivariate Cox regression of available risk factors in the NKI and Wang et al. data sets was performed (FIG. 9 a, b). Although the mixed-outcome group was enriched for ER-positive/HER2-negative tumors, the good and poor outcome groups identified by the SDPP were composed of tumors with mixed ER and HER2 status (FIG. 9 c). In addition, the SDPP identifies good vs. poor outcome patients in both ER-positive and HER2-positive patient cohorts (FIG. 4 b, c, e, dashed lines, parentheses). In multivariate analyses, the SDPP was independent of classical clinical risk factors including ER and HER2 status, lymph node involvement, grade and age (FIG. 9 a, b), demonstrating that the SDPP is a novel predictor that identifies patients at risk of relapse independent of classical clinical risk factors.

### The SDPP is Independent of Previously Described Predictors and Signatures

Other expression-based prognostic signatures and predictors have been identified in breast cancer³. The 70-gene predictor of van't Veer et. al.³ developed from a subset of the NKI patient cohort, has received FDA market clearance for use as a predictor for metastatic progression. Genes within this predictor have been identified as involved in proliferation, angiogenesis, and invasion^{3,37}. In addition, signatures have been developed that reflect biological responses in vitro^{19,20}. For example, the concept of tumors as “wounds that do not heal” led to the identification of a wound response signature derived from the response of stromal fibroblasts in culture to serum stimulation²⁰. Similarly, since tumors undergo adaptation to hypoxia in response to decreased oxygen, a hypoxia-associated transcriptional response was derived from cell culture studies¹⁹. Interestingly, both of these signatures can predict outcome in different cancer types^{19,20}.

To test how the SDPP performs when compared to other predictors and signatures, the NKI dataset, where both the wound and hypoxia signatures predict outcome was examined^{19,20}. Multivariate Cox regression showed that, despite some correlation (FIG. 9 d), the SDPP was independent of the wound response and hypoxia signatures (Table 6), demonstrating that the SDPP reflects important biological processes beyond these signatures. One gene present in the hypoxia signature (ADM) is present within the SDPP, thus implicating hypoxia as an important component of the SDPP.

Additionally, the SDPP was independent of, and outperformed, the 70-gene predictor in the HER2-positive cohort of the NKI data (FIG. 9 a, e). These results demonstrate that the SDPP provides additional information to predict outcome, independent of published stroma-associated signatures and predictors entering clinical use.

### Discussion

While there is an increasing awareness that stromal interactions contribute to tumor progression, the role played by the microenvironment in primary breast cancers is poorly understood. Previous predictors have not specifically investigated the biological processes that occur in stroma. Such insight is essential for the development of new therapeutic strategies. SDPP, based on differential gene expression patterns in tumor-associated stroma, forecasts disease outcome with greater accuracy than do predictors based on whole tissue, suggesting that gene expression in tumor associated stroma modulates progression and outcome. Multiple biological responses are differentially reflected within the stroma of patients in different outcome categories.

Tumor associated stroma samples comprising the good-outcome patient cluster (FIG. 2a) overexpress a distinct set of immune-related genes relative to the other clusters, including T-cell and NK-cell markers indicative of a Th1-type immune response. This is consistent with previous work reporting a correlation between increased memory Th1 cell content and good outcome in colon cancer³⁸. In contrast, this response is significantly diminished in patients of the poor outcome cluster (FIG. 2a). Stroma from poor outcome patients exhibits elevated expression of macrophage chemoattractants and macrophage scavenger receptors (FIG. 8a), supporting a Th2-type immune response³⁹′⁴°. This is associated with poor outcome in animal models of breast cancer, including the polyoma middle-T model where type II macrophages stimulate invasion and metastasis by tumor cells⁴¹⁻⁴³.

Type II macrophages can be recruited to the tumor microenvironment via hypoxia. An elevated expression of the transcription factor HIF1A (hypoxia inducible factor 1-alpha), as well as VEGF (vascular endothelial growth factor), and EDN2 (endothelin 2) was observed in the poor-outcome vs. good-outcome clusters (FIG. 8 a). VEGF, CXCL1 and EDN2 are chemoattractants able to recruit monocytes to the tumor site⁴⁰, where they may differentiate into type II macrophages. Two additional genes elevated in this cluster of patients, MSR1 (macrophage scavenger receptor 1) and MARCO (macrophage scavenger receptor with collagenous structure) (FIG. 8 a), are markers of type II macrophages^{40,44}. In addition, the poor-outcome patient cluster exhibits increased markers for endothelial cells (FIG. 8 b-d), confirming previous reports that increased blood vessel density correlates with poor clinical outcome^{45,46}. A significantly higher blood vessel density in tissues from patients was observed in the mixed and poor clusters vs. patients in the good cluster (FIG. 1a).

The increased expression of pro-angiogenic factors as well as enrichment for other angiogenesis-related genes such as VEGF and EDN2 in the poor outcome cluster of patients supports a role for this process in affecting breast cancer outcome.

Although each of these biological responses (differential immune response, hypoxia and angiogenesis) has previously been associated with poor prognosis, their value as independent prognostic factors remains in question^{31,32}. This study reveals that integrating the output of these processes generates an independent predictor of outcome. In particular, one component of the SDPP, representing hypoxia and angiogenesis, is associated with poor outcome, while another, representing a specific immune response, is associated with good outcome.

Osteopontin (SPP1) expression is strongly associated with the poor-outcome group in both the NKI and Wang et al. data sets. Increased immunostaining of breast carcinoma cells for this protein has previously been associated with poor outcome⁴⁷, and is also observed in members of our patient cohort (FIG. 5 c)

The stroma-derived pattern of gene expression, distilled as a 26-gene set is a robust predictor; it is correlated with clinical outcome in public breast cancer datasets derived from whole tumor tissue, using a subset of the 26 genes for outcome prediction^{12,18} Notably, tumors from good and poor outcome patients identified by the SDPP in the NKI patient data do not segregate by ER or HER2 status (FIG. 9 a, c), indicating that the SDPP identifies distinct biological processes, rather than those associated with known clinical breast cancer subtypes.

Although conventional histological diagnosis and immunohistochemical testing is currently used to identify distinct clinical subtypes of breast cancer, it often fails to classify patients by outcome⁴⁸. The relative risk associated with poor-outcome-associated stroma identified by the SDPP is greater than, and independent of, lymph node involvement, the current gold standard for predicting outcome in breast cancer⁴⁹ (Table 6, FIG. 9 a). Interestingly, the SDPP shows significantly increased relative risk in HER2-positive patients (FIG. 4 b, c, e). This is consistent with reports demonstrating a link between HER2-positive human breast cancer and increased angiogenesis⁵°

A predictor of outcome for breast cancer derived from gene expression signatures⁵¹ has recently received FDA market clearance. The SDPP gene set shows no overlap and adds independent information to this 70-gene predictor (Table 6, FIG. 9 a), and, in the data sets examined, outperforms it in HER2-positive patients, providing increased accuracy (FIG. 9 e). When compared with the wound and hypoxia signatures and the 70-gene predictor, our SDPP is the only one of the four that forecasts metastasis or poor outcome with greater than 50% accuracy (FIG. 9 g).

TABLE 1
Clinical characteristics of patients included in the study

| | |
|---|---|
| lymph node status | 25 positive/25 negative/3 not available |
| estrogen receptor status | 43 positive/10 negative |
| grade | 3 grade I/23 grade II/27 grade III |
| HER2 status | 10 positive/43 negative |
| progesterone receptor | 27 positive/26 negative |
| status | |
| mean age of operation | 54.11 years, SD = 11.3 |
| mean tumor size | 22.4 mm, SD = 12.19 |
| stage | 17 I/19 IIA/10 IIB/2 IIIA/2 IIIC/2 other/ |
| | 2 not available |
| recurrence | 41 negative/11 positive/1 not available |
| clinical outcome | 40 disease free/6 alive with disease/2 dead |
| | of disease/1 dead of other causes/4 |
| | untraceable for 3 years |
| median follow up | 3.44 years, SD = 1.69 |
| post op. hormonal TX | 37 yes/11 no/5 not available |
| post op. chemo | 36 yes/12 no/5 not available |
| axillary LN dissection | 47 yes/5 no/1 not available |

TABLE 2
The 200 most variable genes in Tumor-associated versus Normal Stroma

| | | | | |
|---|---|---|---|---|
| SCGB2A2 | AW946823 | GTF2H3 | CD69 | DDX52 |
| BX119435 | LOC118430 | EDIL3 | AK001808 | SPP1 |
| PRG4 | FLJ14167 | MYB | GK | IPP |
| RIPK4 | S100A9 | KLK11 | PDLIM3 | ARHGAP24 |
| DCD | SGCB | CD69 | TLN2 | |
| THC2433234 | BX090412 | RNASE1 | C1GALT1 | |
| IL8 | GALNT3 | THC2375558 | THC2331323 | |
| THC2415754 | COLM | Cep290 | NELL2 | |
| C10orf81 | THC2358845 | CREBL2 | THC2311186 | |
| AK125162 | USP6NL | PDIK1L | MTL5 | |
| LOC400701 | SCRG1 | AL512727 | THC2269657 | |
| S100A8 | MMP3 | SEPT10 | CD69 | |
| DKFZp779O175 | PTPN13 | THC2323620 | BC041996 | |
| PCOLCE2 | LRRN1 | AF161369 | MMP7 | |
| KIAA0853 | TM4SF9 | BCL6 | FLJ30058 | |
| SP5 | THC2317432 | FGF18 | AK025909 | |
| BEX1 | SIGLECP3 | BMP7 | THC2358845 | |
| PRND | POU2AF1 | BAMBI | RNF6 | |
| FLJ10094 | COLEC12 | CD69 | KIAA1799 | |
| THC2290786 | THC2302062 | AK000038 | AK094963 | |
| GPC6 | F2RL2 | NPY2R | ROR1 | |
| THC2276218 | BC042026 | AK091375 | CXCL9 | |
| FLJ11280 | ACADSB | NCF2 | AK095841 | |
| ESR1 | THC2455681 | SCUBE2 | THC2306884 | |
| SCGB2A1 | SLC39A6 | RAPH1 | BM982926 | |
| AREG | CD69 | KIAA1005 | THRSP | |
| THC2289112 | THC2373940 | ENST00000334308 | KIF5C | |
| JAG1 | AK024878 | FLJ14966 | THC2397265 | |
| THC2303268 | GRP | FLJ36492 | BC047014 | |
| HOXA10 | AK026984 | BMP2K | LOC284018 | |
| CCL18 | AK130862 | PLEK | RASGRP3 | |
| DMXL1 | AK021897 | MGC15937 | AK094718 | |
| BM968705 | IQGAP2 | AK025947 | ENST00000331696 | |
| CXCL1 | BF803942 | DKFZp313A2432 | A_32_P58912 | |
| FLJ12787 | TP53I3 | BHLHB5 | CD69 | |
| PLN | CD96 | CD69 | SOX11 | |
| MAWBP | MYOM2 | TRIM36 | KCNMB4 | |
| MGC8685 | DPT | FLJ11588 | DCTD | |
| CD28 | AK123533 | THC2351317 | RAP2B | |
| FLJ31204 | ITGAE | BX097190 | ACTG2 | |
| AA292106 | LOC375251 | ZFP1 | IKIP | |
| DACH1 | MS4A6A | FLJ31340 | PDCL | |
| AF086529 | CD69 | MGC5391 | AK127309 | |
| C6orf117 | RDH10 | A_24_P706752 | KIF1B | |
| VSNL1 | CD24 | AF035031 | MMP12 | |
| GDF6 | THC2351317 | FLJ39485 | ZNF138 | |
| DKFZp434H1419 | CD69 | CD69 | C8orf1 | |
| LTF | FCGBP | ZNF336 | C4orf15 | |
| THC2347909 | FLJ30596 | NUDCD1 | THC2404429 | |

TABLE 3
Genes from class distinction ordered by p-value for recurrence
prediction in multivariate logistic regression.

| GeneName | Description | p value |
|---|---|---|
| THC2394165 | DBP_HUMAN (Q10586) D-site-binding protein | 0.016545147 |
| | (Albumin D box-binding protein) | |
| | (TAXREB302), partial (6%) [THC2394165] | |
| CD52 | Homo sapiens CD52 antigen (CAMPATH-1 | 0.017008396 |
| | antigen) (CD52), mRNA [NM_001803] | |
| SLC40A1 | Homo sapiens solute carrier family 40 (iron- | 0.02013069 |
| | regulated transporter), member 1 | |
| | (SLC40A1), mRNA [NM_014585] | |
| AK055101 | Homo sapiens cDNA FLJ30539 fis, clone | 0.021250406 |
| | BRAWH2001255. [AK055101] | |
| ADM | Homo sapiens adrenomedullin (ADM), mRNA | 0.022394706 |
| | [NM_001124] | |
| GZMA | Homo sapiens granzyme A (granzyme 1, | 0.022655536 |
| | cytotoxic T-lymphocyte-associated | |
| | serine esterase 3) (GZMA), mRNA | |
| | [NM_006144] | |
| RAI2 | Homo sapiens retinoic acid induced 2 (RAI2), | 0.026543837 |
| | mRNA [NM_021785] | |
| HRASLS | Homo sapiens HRAS-like suppressor | 0.027779735 |
| | (HRASLS), mRNA [NM_020386] | |
| F2RL2 | Homo sapiens coagulation factor II | 0.027955989 |
| | (thrombin) receptor-like 2 (F2RL2), mRNA | |
| | [NM_004101] | |
| CD3Z | Homo sapiens CD3Z antigen, zeta polypeptide | 0.028045184 |
| | (TiT3 complex) (CD3Z), | |
| | transcript variant 1, mRNA [NM_198053] | |
| CD48 | Homo sapiens CD48 antigen (B-cell | 0.031863854 |
| | membrane protein) (CD48), mRNA | |
| | [NM_001778] | |
| CD8A | Homo sapiens CD8 antigen, alpha polypeptide | 0.036322667 |
| | (p32) (CD8A), transcript | |
| | variant 1, mRNA [NM_001768] | |
| SPP1 | Homo sapiens secreted phosphoprotein 1 | 0.036662533 |
| | (osteopontin, bone sialoprotein I, | |
| | early T-lymphocyte activation 1) (SPP1), | |
| | mRNA [NM_000582] | |
| VGLL1 | Homo sapiens vestigial like 1 (Drosophila) | 0.037604719 |
| | (VGLL1), mRNA [NM_016267] | |
| OGN | Homo sapiens osteoglycin (osteoinductive | 0.037801701 |
| | factor, mimecan) (OGN), transcript | |
| | variant 1, mRNA [NM_033014] | |
| ADRA2A | Homo sapiens adrenergic, alpha-2A-, receptor | 0.044127358 |
| | (ADRA2A), mRNA [NM_000681] | |
| HOXA10 | Homo sapiens homeo box A10 (HOXA10), | 0.045274517 |
| | transcript variant 1, mRNA [NM_018951] | |
| C21orf34 | Homo sapiens chromosome 21 open reading | 0.046654839 |
| | frame 34 (C21orf34), transcript | |
| | variant 1, mRNA [NM_001005732] | |
| BC028083 | Homo sapiens cDNA clone MGC: 40031 | 0.048160267 |
| | IMAGE: 5217067, complete cds. [BC028083] | |
| GIMAP5 | Homo sapiens GTPase, IMAP family member 5 | 0.049534101 |
| | (GIMAP5), mRNA [NM_018384] | |
| CXCL14 | Homo sapiens chemokine (C-X-C motif) | 0.051205513 |
| | ligand 14 (CXCL14), mRNA [NM_004887] | |
| PLEK | Homo sapiens pleckstrin (PLEK), mRNA | 0.054832724 |
| | [NM_002664] | |
| RUNX3 | Homo sapiens runt-related transcription | 0.057887525 |
| | factor 3 (RUNX3), mRNA [NM_004350] | |
| FRZB | Homo sapiens frizzled-related protein (FRZB), | 0.05872814 |
| | mRNA [NM_001463] | |
| AL359052 | Homo sapiens mRNA full length insert cDNA | 0.065683138 |
| | clone EUROIMAGE 1968422. [AL359052] | |
| LCP1 | Homo sapiens lymphocyte cytosolic protein 1 | 0.075396944 |
| | (L-plastin) (LCP1), mRNA [NM_002298] | |
| ACTG2 | Homo sapiens actin, gamma 2, smooth | 0.076316091 |
| | muscle, enteric (ACTG2), mRNA | |
| | [NM_001615] | |
| PRND | Homo sapiens prion protein 2 (dublet) | 0.080013991 |
| | (PRND), mRNA [NM_012409] | |
| SOAT1 | Homo sapiens sterol O-acyltransferase (acyl- | 0.080257195 |
| | Coenzyme A: cholesterol acyltransferase) | |
| | 1 (SOAT1), transcript variant 688113, mRNA | |
| | [NM_003101] | |
| AI345640 | AI345640 tb83h08.x1 NCI_CGAP_Lu26 | 0.082312362 |
| | Homo sapiens cDNA clone | |
| | IMAGE: 2060991 3′, mRNA sequence | |
| | [AI345640] | |
| LAP3 | Homo sapiens leucine aminopeptidase 3 | 0.082391142 |
| | (LAP3), mRNA [NM_015907] | |
| ESR1 | Homo sapiens estrogen receptor 1 (ESR1), | 0.084286211 |
| | mRNA [NM_000125] | |
| CHEK1 | Homo sapiens CHK1 checkpoint homolog | 0.091069111 |
| | (S. pombe) (CHEK1), mRNA [NM_001274] | |
| THC2436642 | Unknown | 0.09188375 |
| MS4A4A | Homo sapiens membrane-spanning 4- | 0.092046093 |
| | domains, subfamily A, member 4 (MS4A4A), | |
| | transcript variant 1, mRNA [NM_024021] | |
| AREG | Homo sapiens amphiregulin (schwannoma- | 0.094379863 |
| | derived growth factor) (AREG), mRNA | |
| | [NM_001657] | |
| HCAP-G | Homo sapiens chromosome condensation | 0.096270775 |
| | protein G (HCAP-G), mRNA [NM_022346] | |
| RSNL2 | Homo sapiens cDNA: FLJ21069 fis, clone | 0.099914735 |
| | CAS01594. [AK024722] | |
| TLN2 | Homo sapiens talin 2 (TLN2), mRNA | 0.108430157 |
| | [NM_015059] | |
| PIP | Homo sapiens prolactin-induced protein (PIP), | 0.111682893 |
| | mRNA [NM_002652] | |
| MYBL1 | H. sapiens a-myb mRNA. [X66087] | 0.113420682 |
| TRA@ | Homo sapiens T cell receptor alpha locus, | 0.113766311 |
| | mRNA (cDNA clone MGC: 71411 | |
| | IMAGE: 4853814), complete cds. [BC063385] | |
| CD3D | Homo sapiens CD3D antigen, delta | 0.114279702 |
| | polypeptide (TiT3 complex) (CD3D), mRNA | |
| | [NM_000732] | |
| GREB1 | Homo sapiens GREB1 protein (GREB1), | 0.126502159 |
| | transcript variant a, mRNA [NM_014668] | |
| IL8 | Homo sapiens interleukin 8 (IL8), mRNA | 0.128168335 |
| | [NM_000584] | |
| ROPN1 | Homo sapiens mRNA; cDNA DKFZp434B1222 | 0.130210869 |
| | (from clone DKFZp434B1222). [AL133624] | |
| IL4I1 | Homo sapiens interleukin 4 induced 1 (IL4I1), | 0.138908592 |
| | transcript variant 2, mRNA [NM_172374] | |
| RIPK4 | Homo sapiens receptor-interacting serine- | 0.139514813 |
| | threonine kinase 4 (RIPK4), | |
| | mRNA [NM_020639] | |
| MMP12 | Homo sapiens matrix metalloproteinase 12 | 0.142237973 |
| | (macrophage elastase) (MMP12), | |
| | mRNA [NM_002426] | |
| ASPM | Homo sapiens asp (abnormal spindle)-like, | 0.145153886 |
| | microcephaly associated (Drosophila) | |
| | (ASPM), mRNA [NM_018136] | |
| TFEC | Homo sapiens transcription factor EC (TFEC), | 0.153557195 |
| | transcript variant 1, mRNA [NM_012252] | |
| GK | Homo sapiens glycerol kinase (GK), transcript | 0.154566723 |
| | variant 1, mRNA [NM_203391] | |
| RaLP | Homo sapiens rai-like protein (RaLP), mRNA | 0.156466627 |
| | [NM_203349] | |
| SLPI | Homo sapiens secretory leukocyte protease | 0.16590614 |
| | inhibitor (antileukoproteinase) (SLPI), | |
| | mRNA [NM_003064] | |
| UBE2C | Homo sapiens ubiquitin-conjugating enzyme | 0.172867806 |
| | E2C (UBE2C), transcript variant 6, | |
| | mRNA [NM_181803] | |
| ENST00000327788 | Homo sapiens cDNA clone IMAGE: 6616931, | 0.173983574 |
| | partial cds. [BC062748] | |
| CF529502 | CF529502 UI-1-BC1p-ash-d-10-0-UI.s1 | 0.176622581 |
| | NCI_CGAP_Pl3 Homo sapiens cDNA | |
| | clone UI-1-BC1p-ash-d-10-0-UI 3′, mRNA | |
| | sequence [CF529502] | |
| XCL1 | Homo sapiens chemokine (C motif) ligand 1 | 0.184641728 |
| | (XCL1), mRNA [NM_002995] | |
| LOC146909 | Homo sapiens hypothetical protein | 0.189068667 |
| | LOC146909, mRNA (cDNA clone | |
| | IMAGE: 4587138), | |
| | partial cds. [BC067365] | |
| CALB2 | Homo sapiens calbindin 2, 29 kDa (calretinin) | 0.189614271 |
| | (CALB2), transcript variant CALB2, | |
| | mRNA [NM_001740] | |
| NM_001017978 | Homo sapiens hypothetical LOC203413 | 0.191287071 |
| | (LOC203413), mRNA [NM_001017978] | |
| COTL1 | Homo sapiens coactosin-like 1 (Dictyostelium) | 0.191310333 |
| | (COTL1), mRNA [NM_021149] | |
| CAPS | Homo sapiens calcyphosine (CAPS), transcript | 0.207901789 |
| | variant 1, mRNA [NM_004058] | |
| CD2 | Homo sapiens CD2 antigen (p50), sheep red | 0.220877524 |
| | blood cell receptor (CD2), | |
| | mRNA [NM_001767] | |
| FAM54A | Homo sapiens family with sequence similarity | 0.224533429 |
| | 54, member A (FAM54A), | |
| | mRNA [NM_138419] | |
| RIOK3 | Homo sapiens RIO kinase 3 (yeast) (RIOK3), | 0.224929935 |
| | transcript variant 2, mRNA [NM_145906] | |
| BC042028 | Homo sapiens, clone IMAGE: 4794726, mRNA. | 0.230783206 |
| | [BC042028] | |
| HCST | Homo sapiens hematopoietic cell signal | 0.234368709 |
| | transducer (HCST), transcript variant 1, | |
| | mRNA [NM_014266] | |
| AR | Homo sapiens androgen receptor | 0.237412841 |
| | (dihydrotestosterone receptor; testicular | |
| | feminization; | |
| | spinal and bulbar muscular atrophy; Kennedy | |
| | disease) (AR), transcript variant 1, mRNA | |
| | [NM_000044] | |
| ENST00000326227 | full-length cDNA clone CS0DI002YD16 of | 0.239257965 |
| | Placenta Cot 25-normalized of | |
| | Homo sapiens (human). [CR603756] | |
| C6orf173 | Homo sapiens chromosome 6 open reading | 0.246856699 |
| | frame 173 (C6orf173), mRNA | |
| | [NM_001012507] | |
| AI659667 | AI659667 tu25d01.x1 NCI_CGAP_Pr28 | 0.248581186 |
| | Homo sapiens cDNA clone | |
| | IMAGE: 2252065 3′ similar to gb: X16940 | |
| | ACTIN, GAMMA-ENTERIC SMOOTH | |
| | MUSCLE (HUMAN);, mRNA sequence | |
| | [AI659667] | |
| STK38L | Homo sapiens serine/threonine kinase 38 like | 0.263901757 |
| | (STK38L), mRNA [NM_015000] | |
| TFF1 | Homo sapiens trefoil factor 1 (breast cancer, | 0.275264605 |
| | estrogen-inducible sequence expressed in) | |
| | (TFF1), mRNA [NM_003225] | |
| PLA2G7 | Homo sapiens phospholipase A2, group VII | 0.27841413 |
| | (platelet-activating factor acetylhydrolase, | |
| | plasma) (PLA2G7), mRNA [NM_005084] | |
| SCRG1 | Homo sapiens scrapie responsive protein 1 | 0.279250645 |
| | (SCRG1), mRNA [NM_007281] | |
| SCEL | Homo sapiens sciellin (SCEL), transcript | 0.289591692 |
| | variant 2, mRNA [NM_144777] | |
| A_24_P828054 | Unknown | 0.305715646 |
| COTL1 | Homo sapiens coactosin-like 1 (Dictyostelium) | 0.313159593 |
| | (COTL1), mRNA [NM_021149] | |
| C1orf38 | Homo sapiens chromosome 1 open reading | 0.319841718 |
| | frame 38 (C1orf38), mRNA [NM_004848] | |
| NCF2 | Homo sapiens neutrophil cytosolic factor 2 | 0.324955902 |
| | (65 kDa, chronic granulomatous disease, | |
| | autosomal 2) (NCF2), mRNA [NM_000433] | |
| SQLE | Homo sapiens squalene epoxidase (SQLE), | 0.33389314 |
| | mRNA [NM_003129] | |
| HLA-A | Homo sapiens major histocompatibility | 0.347666445 |
| | complex, class I, A (HLA-A), mRNA | |
| | [NM_002116] | |
| TFF3 | Homo sapiens trefoil factor 3 (intestinal) | 0.348310603 |
| | (TFF3), mRNA [NM_003226] | |
| MGC40042 | Homo sapiens cDNA FLJ36022 fis, clone | 0.356708975 |
| | TESTI2016599. [AK093341] | |
| GZMB | Homo sapiens granzyme B (granzyme 2, | 0.358908466 |
| | cytotoxic T-lymphocyte-associated serine | |
| | esterase 1) (GZMB), mRNA [NM_004131] | |
| AQP9 | Homo sapiens aquaporin 9 (AQP9), mRNA | 0.363817928 |
| | [NM_020980] | |
| SORCS2 | Homo sapiens sortilin-related VPS10 domain | 0.389697595 |
| | containing receptor 2 (SORCS2), mRNA | |
| | [NM_020777] | |
| FLJ23311 | Homo sapiens likely ortholog of mouse E2F | 0.38981832 |
| | transcription factor 8 (E2F8), mRNA | |
| | [NM_024680] | |
| MMP7 | Homo sapiens matrix metalloproteinase 7 | 0.394624625 |
| | (matrilysin, uterine) (MMP7), mRNA | |
| | [NM_002423] | |
| AW205591 | UI-H-BI1-afr-b-02-0-UI.s1 NCI_CGAP_Sub3 | 0.403726656 |
| | Homo sapiens cDNA | |
| | clone IMAGE: 2722515 3′, mRNA sequence | |
| | [AW205591] | |
| THC2301370 | Q7Z5X4 (Q7Z5X4) Intermediate filament-like | 0.404659448 |
| | protein MGC: 2625, isoform 1, partial (12%) | |
| | [THC2301370] | |
| CCL13 | Homo sapiens chemokine (C-C motif) ligand | 0.406340173 |
| | 13 (CCL13), mRNA [NM_005408] | |
| STK24 | Homo sapiens serine/threonine kinase 24 | 0.415797555 |
| | (STE20 homolog, yeast) (STK24), | |
| | mRNA [NM_003576] | |
| MMP1 | Homo sapiens matrix metalloproteinase 1 | 0.418743075 |
| | (interstitial collagenase) (MMP1), | |
| | mRNA [NM_002421] | |
| TCEA3 | Homo sapiens transcription elongation factor | 0.427745686 |
| | A (SII), 3 (TCEA3), mRNA [NM_003196] | |
| KYNU | Homo sapiens kynureninase (L-kynurenine | 0.431211173 |
| | hydrolase) (KYNU), mRNA [NM_003937] | |
| GBP5 | Homo sapiens guanylate binding protein 5 | 0.438367981 |
| | (GBP5), mRNA [NM_052942] | |
| C6orf117 | Homo sapiens chromosome 6 open reading | 0.439094061 |
| | frame 117 (C6orf117), mRNA [NM_138409] | |
| HTATIP2 | Homo sapiens HIV-1 Tat interactive protein 2, | 0.43939817 |
| | 30 kDa (HTATIP2), mRNA [NM_006410] | |
| C6orf51 | Homo sapiens chromosome 6 open reading | 0.441124499 |
| | frame 51 (C6orf51), mRNA [NM_138408] | |
| SLC30A5 | Homo sapiens solute carrier family 30 (zinc | 0.510666848 |
| | transporter), member 5 (SLC30A5), | |
| | mRNA [NM_022902] | |
| SCGB2A2 | Homo sapiens secretoglobin, family 2A, | 0.511507414 |
| | member 2 (SCGB2A2), mRNA [NM_002411] | |
| OXR1 | Homo sapiens oxidation resistance 1 (OXR1), | 0.517898764 |
| | mRNA [NM_181354] | |
| SRPK1 | Homo sapiens SFRS protein kinase 1 | 0.523708752 |
| | (SRPK1), mRNA [NM_003137] | |
| S100A7 | Homo sapiens S100 calcium binding protein | 0.525332473 |
| | A7 (psoriasin 1) (S100A7), | |
| | mRNA [NM_002963] | |
| CHML | Homo sapiens choroideremia-like (Rab escort | 0.537228179 |
| | protein 2) (CHML), mRNA [NM_001821] | |
| KRT23 | Homo sapiens keratin 23 (histone deacetylase | 0.541169602 |
| | inducible) (KRT23), | |
| | transcript variant 2, mRNA [NM_173213] | |
| DSCR1 | Homo sapiens Down syndrome critical region | 0.551318496 |
| | gene 1 (DSCR1), | |
| | transcript variant 1, mRNA [NM_004414] | |
| GRB14 | Homo sapiens growth factor receptor-bound | 0.556768881 |
| | protein 14 (GRB14), mRNA [NM_004490] | |
| EDN1 | Homo sapiens endothelin 1 (EDN1), mRNA | 0.568485024 |
| | [NM_001955] | |
| S100A8 | Homo sapiens S100 calcium binding protein | 0.576001393 |
| | A8 (calgranulin A) | |
| | (S100A8), mRNA [NM_002964] | |
| NM_001012985 | Homo sapiens chromosome 1 open reading | 0.582299498 |
| | frame 31 (C1orf31), mRNA | |
| | [NM_001012985] | |
| AK024292 | Homo sapiens cDNA FLJ14230 fis, clone | 0.592882221 |
| | NT2RP3004349. [AK024292] | |
| HIST1H1C | Homo sapiens histone 1, H1c (HIST1H1C), | 0.598592114 |
| | mRNA [NM_005319] | |
| BQ186674 | UI-E-EJ1-ajr-f-10-0-UI.r1 UI-E-EJ1 | 0.600394237 |
| | Homo sapiens cDNA clone | |
| | UI-E-EJ1-ajr-f-10-0-UI 5′, mRNA sequence | |
| | [BQ186674] | |
| ZNF165 | Homo sapiens zinc finger protein 165 | 0.610071395 |
| | (ZNF165), mRNA [NM_003447] | |
| AK025522 | Homo sapiens cDNA: FLJ21869 fis, clone | 0.610675911 |
| | HEP02442. [AK025522] | |
| APG5L | Homo sapiens APG5 autophagy 5-like | 0.623017873 |
| | (S. cerevisiae) (APG5L), mRNA [NM_004849] | |
| PERP | Homo sapiens PERP, TP53 apoptosis effector | 0.629694567 |
| | (PERP), mRNA [NM_022121] | |
| C6orf203 | Homo sapiens chromosome 6 open reading | 0.673959886 |
| | frame 203 (C6orf203), mRNA [NM_016487] | |
| CLEC4E | Homo sapiens C-type lectin domain family 4, | 0.680597492 |
| | member E (CLEC4E), mRNA [NM_014358] | |
| CX40.1 | Homo sapiens connexin40.1, mRNA (cDNA | 0.681860214 |
| | clone IMAGE: 5240397), | |
| | partial cds. [BC035898] | |
| TACSTD1 | Homo sapiens tumor-associated calcium | 0.68241981 |
| | signal transducer 1 (TACSTD1), | |
| | mRNA [NM_002354] | |
| OAZIN | Homo sapiens antizyme inhibitor 1 (AZIN1), | 0.693398845 |
| | transcript variant 1, mRNA [NM_015878] | |
| CXCL1 | Homo sapiens chemokine (C-X-C motif) | 0.699703266 |
| | ligand 1 (melanoma growth stimulating | |
| | activity, alpha) (CXCL1), mRNA [NM_001511] | |
| S100A9 | Homo sapiens S100 calcium binding protein | 0.705332192 |
| | A9 (calgranulin B) (S100A9), | |
| | mRNA [NM_002965] | |
| GPR56 | Homo sapiens G protein-coupled receptor 56 | 0.708149135 |
| | (GPR56), transcript variant 3, | |
| | mRNA [NM_201525] | |
| IL10RA | Homo sapiens interleukin 10 receptor, alpha | 0.710303013 |
| | (IL10RA), mRNA [NM_001558] | |
| RDH10 | Homo sapiens retinol dehydrogenase 10 (all- | 0.716306889 |
| | trans) (RDH10), mRNA [NM_172037] | |
| B3GNT5 | Homo sapiens UDP-GlcNAc: betaGal beta-1,3- | 0.737561834 |
| | N-acetylglucosaminyltransferase 5 | |
| | (B3GNT5), mRNA [NM_032047] | |
| UGCGL1 | Homo sapiens UDP-glucose ceramide | 0.737683508 |
| | glucosyltransferase-like 1 (UGCGL1), | |
| | mRNA [NM_020120] | |
| ENST00000246228 | Unknown | 0.766220288 |
| WISP2 | Homo sapiens WNT1 inducible signaling | 0.790569616 |
| | pathway protein 2 (WISP2), mRNA | |
| | [NM_003881] | |
| SUSD3 | Homo sapiens sushi domain containing 3 | 0.793439767 |
| | (SUSD3), mRNA [NM_145006] | |
| FLJ30046 | Homo sapiens hypothetical protein FLJ30046 | 0.799409749 |
| | (FLJ30046), mRNA [NM_144595] | |
| ZHX2 | Homo sapiens zinc fingers and homeoboxes 2 | 0.802456703 |
| | (ZHX2), mRNA [NM_014943] | |
| GPR110 | Homo sapiens G protein-coupled receptor 110 | 0.81582725 |
| | (GPR110), transcript variant 1, | |
| | mRNA [NM_153840] | |
| PDCD7 | Homo sapiens programmed cell death 7 | 0.823759514 |
| | (PDCD7), mRNA [NM_005707] | |
| PSCD3 | Homo sapiens pleckstrin homology, Sec7 and | 0.832910009 |
| | coiled-coil domains 3 (PSCD3), | |
| | mRNA [NM_004227] | |
| KIAA1764 | Homo sapiens KIAA1764 protein (KIAA1764), | 0.834686275 |
| | mRNA [NM_033402] | |
| HLA-F | Homo sapiens major histocompatibility | 0.842207274 |
| | complex, class I, F (HLA-F), mRNA | |
| | [NM_018950] | |
| LACTB2 | Homo sapiens lactamase, beta 2 (LACTB2), | 0.847445642 |
| | mRNA [NM_016027] | |
| CYBB | Homo sapiens cytochrome b-245, beta | 0.85387856 |
| | polypeptide (chronic granulomatous disease) | |
| | (CYBB), mRNA [NM_000397] | |
| OIP5 | Homo sapiens Opa interacting protein 5 | 0.856962272 |
| | (OIP5), mRNA [NM_007280] | |
| CTSL2 | Homo sapiens cathepsin L2 (CTSL2), mRNA | 0.857611057 |
| | [NM_001333] | |
| CENPF | Homo sapiens centromere protein F, | 0.885491602 |
| | 350/400ka (mitosin) (CENPF), mRNA | |
| | [NM_016343] | |
| THC2269172 | Unknown | 0.88738879 |
| S100P | Homo sapiens S100 calcium binding protein P | 0.892778358 |
| | (S100P), mRNA [NM_005980] | |
| LOC124976 | Homo sapiens, Similar to spinster-like | 0.896755882 |
| | protein, clone IMAGE: 4814561, mRNA, partial | |
| | cds. [BC041772] | |
| ECT2 | Homo sapiens epithelial cell transforming | 0.899598815 |
| | sequence 2 oncogene (ECT2), mRNA | |
| | [NM_018098] | |
| HSPC159 | Homo sapiens HSPC159 protein (HSPC159), | 0.899720856 |
| | mRNA [NM_014181] | |
| KNTC2 | Homo sapiens kinetochore associated 2 | 0.917493076 |
| | (KNTC2), mRNA [NM_006101] | |
| CDCA7 | Homo sapiens cell division cycle associated 7 | 0.922005167 |
| | (CDCA7), transcript variant 1, | |
| | mRNA [NM_031942] | |
| LCN2 | Homo sapiens lipocalin 2 (oncogene 24p3) | 0.922267973 |
| | (LCN2), mRNA [NM_005564] | |
| GALNT3 | Homo sapiens UDP-N-acetyl-alpha-D- | 0.929384121 |
| | galactosamine: polypeptide | |
| | N-acetylgalactosaminyltransferase 3 (GalNAc- | |
| | T3) (GALNT3), mRNA [NM_004482] | |
| AMD1 | Homo sapiens adenosylmethionine | 0.936315976 |
| | decarboxylase 1 (AMD1), mRNA | |
| | [NM_001634] | |
| AB209004 | Homo sapiens mRNA for Hypothetical protein | 0.938246637 |
| | DKFZp686O08126 variant protein. | |
| | [AB209004] | |
| ORMDL1 | Homo sapiens ORM1-like 1 (S. cerevisiae) | 0.943061962 |
| | (ORMDL1), mRNA [NM_016467] | |
| CRY1 | Homo sapiens cryptochrome 1 (photolyase- | 0.953083693 |
| | like) (CRY1), mRNA [NM_004075] | |
| FGF18 | Homo sapiens fibroblast growth factor 18 | 0.955558882 |
| | (FGF18), transcript variant 1, mRNA | |
| | [NM_003862] | |
| C20orf129 | Homo sapiens chromosome 20 open reading | 0.960068496 |
| | frame 129 (C20orf129), mRNA | |
| | [NM_030919] | |
| KCNK5 | Homo sapiens potassium channel, subfamily | 0.982098336 |
| | K, member 5 (KCNK5), mRNA | |
| | [NM_003740] | |
| BCAN | Homo sapiens brevican, mRNA (cDNA clone | 0.991036283 |
| | IMAGE: 3618761), partial cds. [BC005081] | |

TABLE 4
LIMMA results for the genes differentially expressed between clusters identified in FIG. 1.

| | | | Cluster 1 | Cluster 1 | Cluster 2 | |
|---|---|---|---|---|---|---|
| | Gene | | vs | vs | vs | adj. |
| RefSeq | Name | TopHit | Cluster 2 | Cluster 3 | Cluster 3 | P. Val |
| NM_002426 | MMP12 | ref\|NM_002426\|gb\| | 0.42926642 | −3.558909454 | −3.988175874 | 4.22E−99 |
| | | L23808\|ens\| | | | | |
| | | ENST00000326227\| | | | | |
| | | gb\|CR603756 | | | | |
| NM_001634 | AMD1 | ref\|NM_001634\|gb\| | 0.32326441 | −1.980177467 | −2.303441877 | 4.83E−99 |
| | | CR599478\|gb\|AK125644\| | | | | |
| | | gb\|AK130474 | | | | |
| NM_001511 | CXCL1 | ref\|NM_001511\|gb\| | 1.136695334 | −3.12014911 | −4.256844444 | 1.31E−94 |
| | | BC011976\|gb\|J03561\| | | | | |
| | | gb\|X12510 | | | | |
| NM_024021 | MS4A4A | ref\|NM_024021\|ref\| | 1.768305052 | 0.499134804 | −1.269170247 | 4.00E−94 |
| | | NM_148975\|gb\|BC020648\| | | | | |
| | | ens\|ENST00000343968 | | | | |
| BC028083 | BC028083 | gb\|BC028083\|gb\|BC071724\| | 1.627723308 | 1.863068798 | 0.23534549 | 1.94E−92 |
| | | gb\|BC036926\|ens\| | | | | |
| | | ENST00000343186 | | | | |
| NM_003137 | SRPK1 | ref\|NM_003137\|gb\|AJ318054\| | 0.415989298 | −1.610928317 | −2.026917614 | 3.28E−85 |
| | | gb\|BC038292\|ens\| | | | | |
| | | ENST00000346162 | | | | |
| NM_005980 | S100P | ref\|NM_005980\|gb\| | 0.732606256 | −2.047775519 | −2.780381775 | 6.04E−84 |
| | | AF539739\|gb\|X65614\| | | | | |
| | | gb\|BC006819 | | | | |
| NM_005707 | PDCD7 | ref\|NM_005707\|gb\| | −0.309571955 | 1.818429443 | 2.128001398 | 1.41E−82 |
| | | AK096970\|gb\|AF083930\| | | | | |
| | | gb\|BC092464 | | | | |
| NM_022121 | PERP | ref\|NM_022121\|gb\| | −0.093190347 | −2.122315468 | −2.029125121 | 1.02E−80 |
| | | AK074585\|gb\|AK097958\| | | | | |
| | | gb\|CR623871 | | | | |
| NM_021149 | COTL1 | ref\|NM_021149\|gb\| | 1.032672352 | −0.512784533 | −1.545456885 | 5.84E−77 |
| | | AK127352\|gb\|BC053682\| | | | | |
| | | gb\|CR625832 | | | | |
| AL133624 | ROPN1 | gb\|AL133624\|ens\| | 0.212201872 | −2.68561337 | −2.897815242 | 9.21E−77 |
| | | ENST00000340906\|gb\| | | | | |
| | | BC067767\|thc\|THC2262630 | | | | |
| NM_002354 | TACSTD1 | ref\|NM_002354\|gb\| | −0.239068254 | −2.13414384 | −1.895075586 | 9.21E−77 |
| | | BC014785\|gb\|CR593061\|gb\| | | | | |
| | | CR626162 | | | | |
| NM_145006 | SUSD3 | ref\|NM_145006\|gb\| | −0.249993569 | 1.735456099 | 1.985449667 | 3.83E−75 |
| | | AK128289\|gb\|AY358190\| | | | | |
| | | gb\|BC014601 | | | | |
| NM_198053 | CD3Z | ref\|NM_198053\|ref\| | 2.320378882 | 1.277252916 | −1.043125966 | 2.12E−74 |
| | | NM_000734\|gb\|AK128376\| | | | | |
| | | gb\|BC025703 | | | | |
| BQ186674 | BQ186674 | gb\|BQ186674\|thc\| | 0.953850405 | −2.575838962 | −3.529689366 | 2.81E−74 |
| | | THC2277801\|thc\| | | | | |
| | | THC2277797\|thc\|THC2277803 | | | | |
| NM_004414 | DSCR1 | ref\|NM_004414\|ref\| | 0.339308079 | −1.66464887 | −2.00395695 | 2.81E−74 |
| | | NM_203418\|ref\|NM_203417\| | | | | |
| | | gb\|AK131569 | | | | |
| NM_006410 | HTATIP2 | ref\|NM_006410\|gb\| | 0.565675559 | −1.610555982 | −2.176231541 | 1.75E−73 |
| | | BC002439\|gb\|U69161\|gb\| | | | | |
| | | AK223010 | | | | |
| NM_004849 | APG5L | ref\|NM_004849\|gb\| | 0.207835011 | −1.846889257 | −2.054724268 | 2.63E−73 |
| | | BX537904\|gb\|Y11588\|ens\| | | | | |
| | | ENST00000360666 | | | | |
| NM_001017978 | NM_001017978 | ref\|NM_001017978\|gb\| | 0.449453002 | −3.129101529 | −3.578554532 | 5.25E−72 |
| | | BC062223\|gb\|AK000618\| | | | | |
| | | gb\|AK026566 | | | | |
| NM_002664 | PLEK | ref\|NM_002664\|gb\| | 1.551703522 | −0.036339873 | −1.588043396 | 2.92E−70 |
| | | BC018549\|gb\|X07743\|gb\| | | | | |
| | | AB208967 | | | | |
| NM_005408 | CCL13 | ref\|NM_005408\|gb\|Z77651\| | 1.742767483 | −0.457950478 | −2.200717962 | 8.09E−70 |
| | | gb\|BC008621\|gb\| | | | | |
| | | U59808 | | | | |
| NM_005084 | PLA2G7 | ref\|NM_005084\|gb\| | 1.572990518 | −0.92568852 | −2.498679038 | 8.64E−70 |
| | | BC038452\|gb\|CR615354\|gb\| | | | | |
| | | CR608325 | | | | |
| NM_005564 | LCN2 | ref\|NM_005564\|gb\| | 0.26235675 | −2.297017504 | −2.559374254 | 2.34E−69 |
| | | CR542092\|gb\|BC033089\| | | | | |
| | | gb\|S75256 | | | | |
| NM_001005732 | C21orf34 | ref\|NM_001005732\|ref\| | −0.071156681 | 1.794381459 | 1.86553814 | 2.62E−69 |
| | | NM_001005733\|ref\| | | | | |
| | | NM_001005734\|gb\| | | | | |
| | | AF486622 | | | | |
| NM_181803 | UBE2C | ref\|NM_181803\|ref\| | 0.722868094 | −1.740998809 | −2.463866904 | 3.03E−69 |
| | | NM_181799\|ref\|NM_007019\| | | | | |
| | | ref\|NM_181800 | | | | |
| NM_000044 | AR | ref\|NM_000044\|ref\| | −0.861061772 | 2.117860777 | 2.978922548 | 4.80E−69 |
| | | NM_001011645\|gb\|M23263\| | | | | |
| | | gb\|M20132 | | | | |
| NM_203349 | RaLP | ref\|NM_203349\|gb\| | −0.04536305 | −3.544413187 | −3.499050137 | 5.06E−68 |
| | | AK124916\|thc\|THC2372273 | | | | |
| NM_001778 | CD48 | ref\|NM_001778\|gb\| | 2.103956026 | 0.82464429 | −1.279311736 | 6.61E−68 |
| | | BC016182\|gb\|X06341\|gb\| | | | | |
| | | M59904 | | | | |
| NM_002965 | S100A9 | ref\|NM_002965\|gb\| | 1.280916459 | −2.382579716 | −3.663496175 | 6.81E−68 |
| | | BC047681\|gb\|CR542224\| | | | | |
| | | gb\|CR542207 | | | | |
| AL359052 | AL359052 | gb\|AL359052\|gb\|AK075136\| | −0.204299393 | 2.613168506 | 2.8174679 | 1.54E−67 |
| | | thc\|THC2373459 | | | | |
| NM_003129 | SQLE | ref\|NM_003129\|gb\|D78130\| | 0.157957829 | −2.096194669 | −2.254152498 | 1.25E−66 |
| | | ens\|ENST00000265896\| | | | | |
| | | gb\|AF098865 | | | | |
| NM_020980 | AQP9 | ref\|NM_020980\|gb\| | 0.699711146 | −2.365377302 | −3.065088448 | 2.65E−66 |
| | | BC026258\|gb\|AB008775\| | | | | |
| | | gb\|AF016495 | | | | |
| NM_018098 | ECT2 | ref\|NM_018098\|gb\| | 0.105397338 | −1.850700027 | −1.956097365 | 9.18E−66 |
| | | AK027713\|gb\|BC006838\| | | | | |
| | | gb\|AY376439 | | | | |
| NM_004848 | C1orf38 | ref\|NM_004848\|gb\| | 1.047757067 | −0.011900337 | −1.059657404 | 3.39E−65 |
| | | AF044896\|gb\|AB050854\| | | | | |
| | | gb\|BC031655 | | | | |
| NM_006144 | GZMA | ref\|NM_006144\|gb\| | 1.715425448 | 1.717155667 | 0.001730219 | 5.60E−65 |
| | | BC015739\|gb\|CR456968\| | | | | |
| | | gb\|M18737 | | | | |
| NM_016343 | CENPF | ref\|NM_016343\|gb\|U19769\| | 0.030520144 | −2.080500292 | −2.111020436 | 7.91E−63 |
| | | ens\|ENST00000271778\| | | | | |
| | | gb\|CR597113 | | | | |
| NM_138408 | C6orf51 | ref\|NM_138408\|gb\| | 0.06038315 | −2.017833649 | −2.078216799 | 2.70E−62 |
| | | AF361492\|gb\|AK057977\| | | | | |
| | | gb\|CR615266 | | | | |
| NM_000584 | IL8 | ref\|NM_000584\|gb\| | −0.169674506 | −4.229297018 | −4.059622512 | 1.78E−61 |
| | | BC013615\|ens\| | | | | |
| | | ENST00000307407\|gb\|Y00787 | | | | |
| NM_015907 | LAP3 | ref\|NM_015907\|gb\| | 1.053565863 | −0.329292253 | −1.382858115 | 2.04E−61 |
| | | AF061738\|gb\|CR605730\| | | | | |
| | | gb\|CR604573 | | | | |
| NM_016267 | VGLL1 | ref\|NM_016267\|gb\| | 0.13739122 | −2.369833242 | −2.507224461 | 4.15E−61 |
| | | BC000045\|gb\|CR590059\| | | | | |
| | | gb\|CR611631 | | | | |
| ENST00000326227 | ENST00000326227 | ens\|ENST00000326227\| | 0.282115338 | −3.569614449 | −3.851729788 | 6.56E−61 |
| | | gb\|CR603756\|gb\| | | | | |
| | | CR594246\|thc\|THC2416644 | | | | |
| NM_002963 | S100A7 | ref\|NM_002963\|gb\| | 0.386915442 | −2.085890313 | −2.472805755 | 8.47E−60 |
| | | BC034687\|gb\|CR542164\| | | | | |
| | | gb\|M86757 | | | | |
| NM_007281 | SCRG1 | ref\|NM_007281\|gb\| | 0.776992451 | −2.706111252 | −3.483103703 | 1.07E−59 |
| | | AJ224677\|gb\|BC017583\| | | | | |
| | | gb\|AY359040 | | | | |
| BC041772 | LOC124976 | gb\|BC041772\|ens\| | 0.497165174 | −1.948190457 | −2.445355631 | 7.25E−59 |
| | | ENST00000329078\|gb\| | | | | |
| | | BC065221\|thc\|THC2435403 | | | | |
| NM_000433 | NCF2 | ref\|NM_000433\|gb | 1.517122409 | −0.888521689 | −2.405644098 | 1.05E−58 |
| | | \|CR593013\|gb\| | | | | |
| | | AB209647\|gb\|M32011 | | | | |
| NM_015000 | STK38L | ref\|NM_015000\|gb\| | −0.067764179 | −2.378935215 | −2.311171036 | 4.01E−57 |
| | | BC028603\|gb\|AB023182\| | | | | |
| | | ens\|ENST00000282893 | | | | |
| NM_020120 | UGCGL1 | ref\|NM_020120\|gb\| | 0.862760333 | −1.589600074 | −2.452360407 | 5.16E−57 |
| | | AK023671\|gb\|AF227905\| | | | | |
| | | gb\|AK074251 | | | | |
| NM_004350 | RUNX3 | ref\|NM_004350\|gb\| | 1.545197628 | 0.725335752 | −0.819861875 | 9.72E−57 |
| | | Z35278\|thc\|THC2337057 | | | | |
| NM_004131 | GZMB | ref\|NM_004131\|gb\| | 1.569404713 | 0.363075418 | −1.206329295 | 2.76E−56 |
| | | AY232655\|gb\|AY232656\| | | | | |
| | | gb\|AY372494 | | | | |
| NM_001955 | EDN1 | ref\|NM_001955\|gb\| | −0.019333113 | −2.234534393 | −2.215201279 | 4.05E−55 |
| | | BC009720\|gb\|CR605456\| | | | | |
| | | gb\|CR591383 | | | | |
| NM_000582 | SPP1 | ref\|NM_000582\|gb\| | −0.308206301 | −2.573947421 | −2.26574112 | 5.23E−55 |
| | | BC022844\|ens\| | | | | |
| | | ENST00000237623\|ens\| | | | | |
| | | ENST00000359072 | | | | |
| NM_173213 | KRT23 | ref\|NM_173213\|ref\| | 0.367603648 | −1.881797814 | −2.249401462 | 5.46E−55 |
| | | NM_015515\|gb\|AL117538\| | | | | |
| | | gb\|AF102848 | | | | |
| NM_052942 | GBP5 | ref\|NM_052942\|gb\| | 1.608826604 | −1.352236459 | −2.961063063 | 4.01E−54 |
| | | AF430642\|gb\|BC033761\| | | | | |
| | | gb\|AF288815 | | | | |
| NM_001124 | ADM | ref\|NM_001124\|gb\|BC015961 | 0.146104901 | −1.904384843 | −2.050489743 | 4.43E−54 |
| | | \|gb\|CR599806\|gb\| | | | | |
| | | CR619368 | | | | |
| NM_003862 | FGF18 | ref\|NM_003862\|gb\| | −1.274239925 | 1.679999573 | 2.954239498 | 1.85E−53 |
| | | AF075292\|gb\|BC006245\| | | | | |
| | | thc\|THC2238947 | | | | |
| NM_003101 | SOAT1 | ref\|NM_003101\|gb\| | 1.229913468 | −0.427938465 | −1.657851933 | 2.82E−53 |
| | | BC028940\|gb\|AL833625\| | | | | |
| | | thc\|THC2258038 | | | | |
| NM_001012507 | C6orf173 | ref\|NM_001012507\|gb\| | 0.51914806 | −1.735833415 | −2.254981475 | 4.54E−52 |
| | | AY902475\|gb\|BC046178\| | | | | |
| | | gb\|BC039556 | | | | |
| NM_004101 | F2RL2 | ref\|NM_004101\|gb\| | 0.21330516 | 2.430313587 | 2.217008427 | 5.24E−52 |
| | | BX537386\|thc\|THC2238152\| | | | | |
| | | thc\|THC2441800 | | | | |
| NM_018136 | ASPM | ref\|NM_018136\|gb\| | −0.000445549 | −1.772766117 | −1.772320568 | 6.63E−52 |
| | | AY367065\|ens\| | | | | |
| | | ENST00000294732\| | | | | |
| | | gb\|AF509326 | | | | |
| NM_003576 | STK24 | ref\|NM_003576\|gb\| | 0.415760143 | −1.110311649 | −1.526071792 | 9.03E−52 |
| | | BC035578\|gb\|CR594698\| | | | | |
| | | gb\|AF024636 | | | | |
| BC042028 | BC042028 | gb\|BC042028\|thc\| | −0.75040035 | −2.678010574 | −1.927610224 | 9.67E−52 |
| | | THC2268868 | | | | |
| NM_014181 | HSPC159 | ref\|NM_014181\|gb\| | −0.019206333 | −2.423379944 | −2.40417361 | 1.44E−51 |
| | | BC062691\|gb\|AF161508\| | | | | |
| | | thc\|THC2336632 | | | | |
| NM_021149 | COTL1 | ref\|NM_021149\|gb\| | 0.75367668 | −0.496101915 | −1.249778594 | 1.64E−51 |
| | | AK127352\|gb\|BC053682\| | | | | |
| | | gb\|CR625832 | | | | |
| NM_020386 | HRASLS | ref\|NM_020386\|gb\| | −0.045751046 | −2.094814827 | −2.049063781 | 2.18E−50 |
| | | AB030816\|gb\|AY251533\| | | | | |
| | | gb\|BC048095 | | | | |
| NM_007280 | OIP5 | ref\|NM_007280\|gb\| | 0.197762581 | −1.354777567 | −1.552540148 | 2.88E−50 |
| | | BC015050\|gb\|CR603974\|gb\| | | | | |
| | | AF025441 | | | | |
| NM_012252 | TFEC | ref\|NM_012252\|ref\| | 0.486962882 | −1.988485282 | −2.475448164 | 7.14E−50 |
| | | NM_001018058\|gb\|CR933605\| | | | | |
| | | gb\|BX538223 | | | | |
| NM_001657 | AREG | ref\|NM_001657\|gb\| | 3.74E−05 | 1.883249949 | 1.883212597 | 1.69E−49 |
| | | BC009799\|gb\|CR617114\| | | | | |
| | | gb\|CR606995 | | | | |
| NM_000732 | CD3D | ref\|NM_000732\|gb\| | 1.763958663 | 1.40406223 | −0.359896432 | 2.60E−49 |
| | | CR599233\|gb\|CR611428\| | | | | |
| | | gb\|BC070321 | | | | |
| AK025522 | AK025522 | gb\|AK025522\|gb\| | 0.29818562 | −1.981997197 | −2.280182817 | 3.95E−49 |
| | | BC047655\|gb\|BC062361\| | | | | |
| | | gb\|XM_495961 | | | | |
| ENST00000246228 | ENST00000246228 | ens\|ENST00000246228 | 0.154753031 | −1.801020651 | −1.955773682 | 5.98E−49 |
| NM_002421 | MMP1 | ref\|NM_002421\|gb\| | 0.412618231 | −1.717574623 | −2.130192854 | 7.87E−49 |
| | | BC013118\|gb\|BC013875\| | | | | |
| | | gb\|AK223035 | | | | |
| NM_172037 | RDH10 | ref\|NM_172037\|gb\| | 0.828099036 | −1.802107849 | −2.630206884 | 9.91E−49 |
| | | BC067131\|thc\|THC2259164 | | | | |
| NM_016467 | ORMDL1 | ref\|NM_016467\|gb\| | 0.119749102 | −2.331777821 | −2.451526923 | 1.02E−48 |
| | | AK075160\|gb\|CR602229\| | | | | |
| | | gb\|AK074756 | | | | |
| NM_016487 | C6orf203 | ref\|NM_016487\|gb\| | 0.399809562 | −1.686621827 | −2.086431388 | 1.07E−48 |
| | | BC010899\|gb\|CR622886\| | | | | |
| | | gb\|AK091564 | | | | |
| NM_172374 | IL4I1 | ref\|NM_172374\|ref\| | 0.734165006 | −1.591497482 | −2.325662487 | 3.37E−48 |
| | | NM_152899\|gb\|BC064378\| | | | | |
| | | gb\|AY358933 | | | | |
| NM_003881 | WISP2 | ref\|NM_003881\|gb\| | −0.108908788 | 2.037769429 | 2.146678217 | 5.19E−48 |
| | | BC017782\|gb\|BC058074\| | | | | |
| | | gb\|AK129660 | | | | |
| NM_003226 | TFF3 | ref\|NM_003226\|gb\| | −0.381250611 | 2.005907116 | 2.387157727 | 1.46E−47 |
| | | BC017859\|ens\| | | | | |
| | | ENST00000291525\|gb\|L08044 | | | | |
| NM_033014 | OGN | ref\|NM_033014\|ref\| | 0.085474785 | 2.128389663 | 2.042914878 | 2.32E−47 |
| | | NM_024416\|ref\|NM_014057\| | | | | |
| | | gb\|AF173383 | | | | |
| NM_003937 | KYNU | ref\|NM_003937\|gb\| | 0.715559367 | −1.649898281 | −2.365457648 | 2.87E−47 |
| | | U57721\|gb\|CR457423\|gb\| | | | | |
| | | CR609484 | | | | |
| NM_001803 | CD52 | ref\|NM_001803\|gb\| | 1.294748778 | 1.621942198 | 0.327193421 | 3.84E−47 |
| | | BC000644\|ens\|ENST00000305548\| | | | | |
| | | gb\|X62466 | | | | |
| NM_144777 | SCEL | ref\|NM_144777\|ref\| | −0.06833074 | −1.774410805 | −1.70608066 | 4.97E−47 |
| | | NM_003843\|gb\|AF045941\| | | | | |
| | | gb\|AK025320 | | | | |
| NM_015878 | OAZIN | ref\|NM_015878\|ref\| | 0.624393081 | −1.217326249 | −1.841719329 | 9.34E−47 |
| | | NM_148174\|gb\|BC013420\| | | | | |
| | | gb\|D88674 | | | | |
| NM_018384 | GIMAP5 | ref\|NM_018384\|gb\| | 1.793308965 | 1.034290439 | −0.759018526 | 1.14E−46 |
| | | AK055568\|gb\|CR594804\| | | | | |
| | | gb\|CR614401 | | | | |
| NM_201525 | GPR56 | ref\|NM_201525\|ref\| | 0.009303847 | −1.730916976 | −1.740220823 | 1.22E−46 |
| | | NM_201524\|ref\|NM_005682\| | | | | |
| | | gb\|AK131550 | | | | |
| NM_001615 | ACTG2 | ref\|NM_001615\|gb\| | −0.486989548 | −2.673425648 | −2.1864361 | 3.61E−46 |
| | | BC012617\|gb\|AK124338\| | | | | |
| | | gb\|X16940 | | | | |
| NM_000125 | ESR1 | ref\|NM_000125\|gb\| | −0.773662917 | 2.313040428 | 3.086703345 | 6.90E−46 |
| | | X03635\|thc\|THC2337408 | | | | |
| AB209004 | AB209004 | gb\|AB209004\|gb\| | −0.417788831 | −2.365675565 | −1.947886734 | 7.23E−46 |
| | | AK054627\|thc\|THC2312426 | | | | |
| THC2394165 | THC2394165 | thc\|THC2394165\|thc\| | −0.03263897 | −2.492640453 | −2.460001483 | 1.84E−45 |
| | | THC2341816 | | | | |
| NM_003064 | SLPI | ref\|NM_003064\|gb\| | 0.30674125 | −1.719234411 | −2.025975661 | 2.88E−45 |
| | | BC020708\|gb\|X04470\| | | | | |
| | | gb\|AX772818 | | | | |
| NM_002298 | LCP1 | ref\|NM_002298\|gb\| | 0.9432857 | −0.39434978 | −1.33763548 | 9.28E−45 |
| | | BC010271\|gb\|CR617209\| | | | | |
| | | gb\|CR593418 | | | | |
| NM_138419 | FAM54A | ref\|NM_138419\|gb\| | 0.339140512 | −1.981917115 | −2.321057627 | 4.46E−44 |
| | | AK125758\|gb\|BC063688\| | | | | |
| | | gb\|BC011716 | | | | |
| NM_004887 | CXCL14 | ref\|NM_004887\|gb\| | −0.141790379 | 2.187252013 | 2.329042393 | 1.00E−43 |
| | | AY358906\|gb\|AF144103\| | | | | |
| | | gb\|AF106911 | | | | |
| NM_012409 | PRND | ref\|NM_012409\|gb\| | −0.217447565 | 2.13208027 | 2.349527835 | 1.06E−43 |
| | | BC043644\|gb\|AF086354\| | | | | |
| | | thc\|THC2273890 | | | | |
| NM_002995 | XCL1 | ref\|NM_002995\|ref\| | 1.339351484 | 0.515793884 | −0.8235576 | 2.37E−43 |
| | | NM_003175\|gb\|D43768\| | | | | |
| | | gb\|U23772 | | | | |
| NM_145906 | RIOK3 | ref\|NM_145906\|ref\| | 0.230098792 | −1.769814637 | −1.999913429 | 9.30E−43 |
| | | NM_003831\|gb\|BC039729\| | | | | |
| | | gb\|AF013591 | | | | |
| NM_003447 | ZNF165 | ref\|NM_003447\|gb\| | 0.198824592 | −1.719421541 | −1.918246134 | 1.15E−42 |
| | | U78722\|gb\|X84801\| | | | | |
| | | gb\|AY366500 | | | | |
| BC067365 | LOC146909 | gb\|BC067365\|gb\|BC044933\| | 0.285360129 | −2.125322975 | −2.410683105 | 1.58E−42 |
| | | ens\|ENST00000335534\| | | | | |
| | | gb\|BC048263 | | | | |
| NM_138409 | C6orf117 | ref\|NM_138409\|gb\| | 0.345196142 | −2.575822953 | −2.921019095 | 2.22E−42 |
| | | AK090775\|gb\|AX746611\| | | | | |
| | | gb\|BC010003 | | | | |
| NM_031942 | CDCA7 | ref\|NM_031942\|ref\| | 0.493574371 | −1.922668313 | −2.416242684 | 5.01E−42 |
| | | NM_145810\|gb\|AL834186\| | | | | |
| | | gb\|AL833728 | | | | |
| AK024292 | AK024292 | gb\|AK024292 | 0.449612338 | −1.997530386 | −2.447142724 | 8.66E−42 |
| NM_020777 | SORCS2 | ref\|NM_020777\|gb\| | −0.102768522 | 2.585220661 | 2.687989183 | 1.19E−41 |
| | | AB037750\|ens\| | | | | |
| | | ENST00000329016\|ens\| | | | | |
| | | ENST00000360303 | | | | |
| AK093341 | MGC40042 | gb\|AK093341\|gb\| | 1.4198565 | 0.946795231 | −0.473061269 | 1.46E−40 |
| | | AK097834\|gb\|AK128158\| | | | | |
| | | gb\|AK094461 | | | | |
| NM_001740 | CALB2 | ref\|NM_001740\|ref\| | −0.245268371 | −2.081911933 | −1.836643562 | 1.62E−40 |
| | | NM_007087\|ref\|NM_007088\| | | | | |
| | | gb\|X56667 | | | | |
| NM_004075 | CRY1 | ref\|NM_004075\|gb\| | 0.398185692 | −1.613099923 | −2.011285615 | 1.84E−40 |
| | | AK098615\|gb\|BC030519\|ens\| | | | | |
| | | ENST00000319645 | | | | |
| NM_001333 | CTSL2 | ref\|NM_001333\|gb\| | 0.278685784 | −1.59782325 | −1.876509034 | 2.85E−40 |
| | | AY358641\|gb\|AF070448\| | | | | |
| | | gb\|Y14734 | | | | |
| NM_000397 | CYBB | ref\|NM_000397\|gb\| | 0.801720517 | −0.325730227 | −1.127450745 | 2.91E−40 |
| | | BC032720\|gb\|X04011\| | | | | |
| | | thc\|THC2245973 | | | | |
| NM_032047 | B3GNT5 | ref\|NM_032047\|gb\| | 0.53075231 | −1.595382453 | −2.126134763 | 3.24E−40 |
| | | AB045278\|gb\|AB209517\| | | | | |
| | | gb\|AK074235 | | | | |
| AI345640 | AI345640 | gb\|AI345640\|gb\| | −0.248359359 | −2.112240723 | −1.863881364 | 3.36E−40 |
| | | AA613834\|gb\|BE138636\| | | | | |
| | | gb\|AI144063 | | | | |
| AK024722 | RSNL2 | gb\|AK024722\|gb\| | 0.304879186 | −1.801005123 | −2.105884309 | 4.24E−40 |
| | | AK057267\|gb\|AF433661\| | | | | |
| | | gb\|BC015310 | | | | |
| NM_016027 | LACTB2 | ref\|NM_016027\|gb\| | 0.346005666 | −1.658379541 | −2.004385207 | 6.96E−40 |
| | | AF151841\|gb\|BC000878\| | | | | |
| | | gb\|BC008505 | | | | |
| NM_004227 | PSCD3 | ref\|NM_004227\|gb\| | −1.032683133 | −0.158794823 | 0.87388831 | 1.56E−39 |
| | | BC008191\|gb\|BC028717\| | | | | |
| | | gb\|AJ223957 | | | | |
| NM_014358 | CLEC4E | ref\|NM_014358\|gb\| | 0.726620708 | −1.521836944 | −2.248457652 | 2.52E−39 |
| | | AB024718\|gb\|BC000715\| | | | | |
| | | gb\|AY358499 | | | | |
| NM_018950 | HLA-F | ref\|NM_018950\|thc\| | 0.991805814 | −0.233573163 | −1.225378977 | 3.55E−39 |
| | | THC2257471 | | | | |
| NM_014585 | SLC40A1 | ref\|NM_014585\|gb\| | 0.339931632 | 2.27719016 | 1.937258528 | 3.85E−39 |
| | | AK002038\|gb\|BC037733\| | | | | |
| | | gb\|AK223236 | | | | |
| NM_001821 | CHML | ref\|NM_001821\|gb\| | 0.094162342 | −2.23799062 | −2.332152962 | 1.13E−38 |
| | | AK023423\|gb\|AK000933\| | | | | |
| | | thc\|THC2336929 | | | | |
| NM_001558 | IL10RA | ref\|NM_001558\|gb\| | 1.008551276 | 0.043160113 | −0.965391164 | 3.89E−38 |
| | | BC028082\|gb\|U00672\| | | | | |
| | | gb\|AB209626 | | | | |
| NM_002964 | S100A8 | ref\|NM_002964\|gb\| | 1.219721065 | −1.635117775 | −2.85483884 | 3.98E−38 |
| | | BC005928\|gb\|X06234\| | | | | |
| | | gb\|Y00278 | | | | |
| AW205591 | AW205591 | gb\|AW205591\|thc\| | 0.096324939 | −1.545968472 | −1.642293411 | 1.10E−37 |
| | | THC2340539 | | | | |
| NM_005319 | HIST1H1C | ref\|NM_005319\|gb\| | 0.281193891 | −1.628203313 | −1.909397204 | 1.19E−37 |
| | | BC002649\|gb\|CR614667\| | | | | |
| | | thc\|THC2256879 | | | | |
| NM_003740 | KCNK5 | ref\|NM_003740\|gb\| | 0.07844286 | −1.191482753 | −1.269925613 | 1.66E−37 |
| | | BC060793\|gb\|AK001897\| | | | | |
| | | gb\|AF084830 | | | | |
| NM_001463 | FRZB | ref\|NM_001463\|gb\| | 0.175296661 | 2.735012294 | 2.559715633 | 8.74E−37 |
| | | U91903\|gb\|BT019883\| | | | | |
| | | gb\|CR593578 | | | | |
| NM_020639 | RIPK4 | ref\|NM_020639\|gb\| | −0.411504416 | −2.286199746 | −1.87469533 | 1.16E−36 |
| | | AL137448\|gb\|AB047783\| | | | | |
| | | ens\|ENST00000352483 | | | | |
| NM_024680 | FLJ23311 | ref\|NM_024680\|gb\| | 0.128740183 | −1.914256449 | −2.042996632 | 3.49E−36 |
| | | AK026964\|gb\|AK055206\| | | | | |
| | | gb\|BC090877 | | | | |
| THC2301370 | THC2301370 | thc\|THC2301370 | −0.320649488 | 2.170531084 | 2.491180573 | 4.19E−36 |
| NM_004482 | GALNT3 | ref\|NM_004482\|gb\| | 0.28317109 | −2.827843813 | −3.111014904 | 6.03E−36 |
| | | BX647473\|thc\|THC2371684 | | | | |
| NM_004058 | CAPS | ref\|NM_004058\|ref\| | 0.275031891 | −2.127671005 | −2.402702897 | 6.72E−36 |
| | | NM_080590\|gb\|AK090469\| | | | | |
| | | gb\|BC011961 | | | | |
| NM_001274 | CHEK1 | ref\|NM_001274\|gb\| | 0.048214662 | −2.116537708 | −2.16475237 | 9.56E−36 |
| | | BC017575\|gb\|AF016582\| | | | | |
| | | gb\|CR591943 | | | | |
| NM_003225 | TFF1 | ref\|NM_003225\|gb\| | −0.281493902 | 1.907728138 | 2.18922204 | 1.97E−35 |
| | | BC032811\|gb\|X52003\| | | | | |
| | | gb\|M12075 | | | | |
| NM_021785 | RAI2 | ref\|NM_021785\|gb\| | −0.016036697 | 1.909846534 | 1.925883231 | 1.89E−34 |
| | | BC027937\|gb\|AK056214\| | | | | |
| | | thc\|THC2299867 | | | | |
| NM_014668 | GREB1 | ref\|NM_014668\|gb\| | −0.047116085 | 1.81045122 | 1.857567305 | 4.01E−34 |
| | | AB011147\|gb\|BC054502\| | | | | |
| | | ens\|ENST00000234142 | | | | |
| NM_001768 | CD8A | ref\|NM_001768\|ref\| | 1.127304112 | 1.541375978 | 0.414071866 | 7.33E−34 |
| | | NM_171827\|gb\|M12824\| | | | | |
| | | gb\|AK124156 | | | | |
| THC2269172 | THC2269172 | thc\|THC2269172 | 0.195226846 | 2.434912409 | 2.239685563 | 1.24E−33 |
| NM_018951 | HOXA10 | ref\|NM_018951\|ref\| | −0.047937912 | 1.835317734 | 1.883255646 | 3.84E−33 |
| | | NM_153715\|gb\|BC013971\| | | | | |
| | | gb\|BC007600 | | | | |
| NM_001767 | CD2 | ref\|NM_001767\|gb\| | 1.446586284 | 0.405313244 | −1.04127304 | 4.90E−33 |
| | | BC033583\|gb\|M16445\| | | | | |
| | | gb\|M16336 | | | | |
| NM_014266 | HCST | ref\|NM_014266\|ref\| | 1.138756239 | −0.697847208 | −0.44090903 | 4.29E−32 |
| | | NM_001007469\|gb\|AY359058\| | | | | |
| | | gb\|BC046348 | | | | |
| NM_022902 | SLC30A5 | ref\|NM_022902\|gb\| | 0.056831204 | −1.850636205 | −1.907467409 | 4.65E−32 |
| | | AK022818\|gb\|BC003411\| | | | | |
| | | gb\|AF212235 | | | | |
| NM_033402 | KIAA1764 | ref\|NM_033402\|gb\| | −0.148514418 | −2.080457968 | −1.93194355 | 1.03E−31 |
| | | BC070092\|gb\|AB051551\| | | | | |
| | | gb\|AK057990 | | | | |
| NM_015059 | TLN2 | ref\|NM_015059\|gb\| | −0.364230712 | 1.648398769 | 2.012629481 | 1.62E−31 |
| | | AF402000\|ens\|ENST00000306829\| | | | | |
| | | thc\|THC2401939 | | | | |
| NM_030919 | C20orf129 | ref\|NM_030919\|gb\| | 0.790953759 | −1.684519889 | −2.475473648 | 1.22E−30 |
| | | AK055793\|gb\|BC063661\| | | | | |
| | | gb\|BC053683 | | | | |
| NM_006101 | KNTC2 | ref\|NM_006101\|gb\| | 0.131342742 | −1.78341244 | −1.914755182 | 2.13E−30 |
| | | BC035617\|gb\|CR609890\| | | | | |
| | | gb\|AF017790 | | | | |
| NM_181354 | OXR1 | ref\|NM_181354\|gb\| | −0.129601586 | −2.10279701 | −1.973195425 | 1.44E−29 |
| | | BC032710\|gb\|AK000987\| | | | | |
| | | gb\|BC035484 | | | | |
| BC063385 | TRA@ | gb\|BC063385\|gb\| | 0.980727986 | 0.997117959 | 0.016389973 | 1.80E−29 |
| | | BC039714\|gb\|BC070344\| | | | | |
| | | gb\|BC070364 | | | | |
| AI659667 | AI659667 | gb\|AI659667\|gb\| | 0.049289282 | −1.13842354 | −1.187712822 | 6.77E−29 |
| | | AA526576\|gb\|AA552462\| | | | | |
| | | gb\|AI749729 | | | | |
| NM_000681 | ADRA2A | ref\|NM_000681\|gb\| | −0.206970872 | 1.819383993 | 2.026354865 | 1.41E−28 |
| | | AF284095\|gb\|BC050414\| | | | | |
| | | thc\|THC2237138 | | | | |
| A_24_P828054 | A_24_P828054 | | 0.138566803 | 1.034265964 | 0.89569916 | 3.35E−28 |
| NM_002423 | MMP7 | ref\|NM_002423\|gb\| | 0.253576833 | −1.912075189 | −2.165652022 | 2.49E−27 |
| | | BC003635\|gb\|X07819\| | | | | |
| | | gb\|AK222980 | | | | |
| NM_002116 | HLA-A | ref\|NM_002116\|gb\| | 0.981558654 | 0.11155115 | −0.870007505 | 2.86E−27 |
| | | M27971\|gb\|U41057\| | | | | |
| | | gb\|X61705 | | | | |
| NM_153840 | GPR110 | ref\|NM_153840\|gb\| | 0.121222896 | −1.780404449 | −1.901627344 | 4.71E−27 |
| | | CR627234\|thc\|THC2366580 | | | | |
| NM_144595 | FLJ30046 | ref\|NM_144595\|gb\| | 0.654428531 | −1.742828249 | −2.39725678 | 6.74E−26 |
| | | AL834203\|gb\|BC045177\| | | | | |
| | | gb\|AL834332 | | | | |
| NM_001012985 | NM_001012985 | ref\|NM_001012985\|gb\| | 0.009865348 | −1.908620306 | −1.918485654 | 1.36E−25 |
| | | BC025793\|ens\|ENST00000302661 | | | | |
| | | \|gb\|CR609197 | | | | |
| NM_004490 | GRB14 | ref\|NM_004490\|gb\| | 0.472486476 | −1.944142699 | −2.416629175 | 1.32E−24 |
| | | L76687\|gb\|AK074599\| | | | | |
| | | gb\|CR612679 | | | | |
| THC2436642 | THC2436642 | thc\|THC2436642 | −0.024943758 | −0.848005574 | −0.823061817 | 4.18E−24 |
| ENST00000327788 | ENST00000327788 | ens\|ENST00000327788\| | 0.018383352 | −1.109362198 | −1.12774555 | 3.56E−23 |
| | | gb\|BC062748\|thc\| | | | | |
| | | THC2433024\|thc\|THC2433022 | | | | |
| NM_014943 | ZHX2 | ref\|NM_014943\|gb\| | −0.012127624 | 0.94731322 | 0.959440844 | 5.30E−23 |
| | | BC042145\|gb\|AB020661\| | | | | |
| | | thc\|THC2246341 | | | | |
| NM_022346 | HCAP-G | ref\|NM_022346\|gb\| | 0.04132144 | −2.077695658 | −2.119017098 | 2.70E−22 |
| | | AF331796\|gb\|AK022512\| | | | | |
| | | gb\|AK023147 | | | | |
| BC005081 | BCAN | gb\|BC005081\|gb\| | −0.225041358 | 1.95921831 | 2.184259668 | 1.40E−21 |
| | | BC067354\|gb\|BC010117\| | | | | |
| | | thc\|THC2276882 | | | | |
| X66087 | MYBL1 | gb\|X66087\|gb\| | 0.099719546 | −1.320123343 | −1.419842889 | 5.86E−21 |
| | | XM_034274\|thc\|THC2336895 | | | | |
| NM_203391 | GK | ref\|NM_203391\|ref\| | 0.309955498 | −1.768537091 | −2.078492589 | 7.33E−21 |
| | | NM_000167\|gb\|BC042421\| | | | | |
| | | gb\|BC071595 | | | | |
| CF529502 | CF529502 | gb\|CF529502\|gb\|BM969871\| | −0.014131643 | 0.845361333 | 0.859492975 | 1.79E−20 |
| | | gb\|AW027319\|gb\| | | | | |
| | | BF513848 | | | | |
| NM_003196 | TCEA3 | ref\|NM_003196\|gb\| | −0.310420044 | 1.664580742 | 1.975000787 | 3.00E−20 |
| | | BC041613\|gb\|AJ223473\| | | | | |
| | | gb\|AK027024 | | | | |
| BC035898 | CX40.1 | gb\|BC035898 | −0.003228725 | −0.829931235 | −0.826702509 | 4.35E−14 |
| AK055101 | AK055101 | gb\|AK055101\|gb\|CR749795\| | −0.144735308 | −0.86696277 | −0.722227462 | 5.21E−11 |
| | | thc\|THC2311974 | | | | |
| NM_002411 | SCGB2A2 | ref\|NM_002411\|gb\| | −0.41161051 | 2.52058094 | 2.93219145 | 5.89E−11 |
| | | U33147\|thc\|THC2239347 | | | | |
| NM_002652 | PIP | ref\|NM_002652\|gb\| | −0.184261606 | 1.74425885 | 1.92852456 | 9.57E−10 |
| | | BC010951\|ens\|ENST00000291009\| | | | | |
| | | gb\|Y10179 | | | | |

TABLE 5
SAM results for the genes differentially expressed between clusters identified in FIG. 1.

| | | | Denominator | | | | |
|---|---|---|---|---|---|---|---|
| Gene ID | Score(d) | Numerator(r) | (s + s0) | contrast-1 | contrast-2 | contrast-3 | q-value(%) |
| VGLL1 | 1.979631557 | 0.732744485 | 0.370141849 | −0.540649753 | −0.954468183 | 6.597220295 | 0 |
| ENST00000326227 | 1.763641998 | 1.115361289 | 0.632419329 | −0.414390175 | −0.889103304 | 5.59217032 | 0 |
| RaLP | 1.757239512 | 1.065058893 | 0.606097738 | −0.614350206 | −0.534480669 | 5.626205552 | 0 |
| CD3Z | 1.752093588 | 1.099042878 | 0.627274071 | 1.4678464 | −2.470735592 | −0.700146669 | 0 |
| BQ186674 | 1.720627099 | 0.986990808 | 0.573622727 | 0.11755293 | −1.663715784 | 4.927805496 | 0 |
| COTL1 | 1.655988908 | 0.552988535 | 0.333932511 | 0.956252427 | −2.53487474 | 2.689809068 | 0 |
| STK24 | 1.620171957 | 0.426865461 | 0.263469232 | 0.126968904 | −1.718099937 | 5.054332584 | 0 |
| AMD1 | 1.609495207 | 0.650161023 | 0.403953376 | −0.227217248 | −1.110887964 | 5.185766014 | 0 |
| ROPN1 | 1.598142529 | 0.839138532 | 0.525071148 | −0.380531181 | −0.816319674 | 5.134780215 | 0 |
| BC028083 | 1.590269335 | 0.857823577 | 0.539420309 | 1.392139128 | −1.854949997 | −2.324432604 | 0 |
| CD48 | 1.589064302 | 0.989001128 | 0.622379551 | 1.28824201 | −2.312904495 | −0.123225181 | 0 |
| CX40.1 | 1.569771991 | 0.250130478 | 0.159341917 | −0.654500388 | −0.627862613 | 6.19263529 | 0 |
| CLEC4E | 1.563567061 | 0.636327541 | 0.406971698 | 0.242646966 | −1.727378086 | 4.368674303 | 0 |
| ENST00000246228 | 1.548614964 | 0.564785682 | 0.364703748 | −0.368281568 | −0.842155042 | 5.146672865 | 0 |
| COTL1 | 1.542369688 | 0.42245539 | 0.273900216 | 0.826847803 | −2.369855866 | 2.931053565 | 0 |
| CD52 | 1.520241375 | 0.702477855 | 0.462083105 | 1.340513206 | −1.713501762 | −2.485275886 | 0 |
| THC2394165 | 1.518555935 | 0.748940499 | 0.493192567 | −0.539762465 | −0.468037793 | 4.937855779 | 0 |
| GPR110 | 1.494517227 | 0.553219083 | 0.370165745 | −0.388756896 | −0.753850518 | 4.97338452 | 0 |
| THC2436642 | 1.49430286 | 0.253530737 | 0.169664894 | −0.669051521 | −0.479410587 | 5.778115426 | 0 |
| MMP12 | 1.487334214 | 1.137974206 | 0.76510995 | −0.272439246 | −0.862921035 | 4.623054842 | 0 |
| GIMAP5 | 1.464043232 | 0.851294567 | 0.581468189 | 1.238254717 | −2.06230502 | −0.665342179 | 0 |
| MS4A4A | 1.454411156 | 0.831863361 | 0.57195887 | 1.150713852 | −2.161801408 | 0.215698904 | 0 |
| NCF2 | 1.450503588 | 0.83273822 | 0.574102834 | 0.751537932 | −2.079075686 | 2.409322154 | 0 |
| SCEL | 1.445004724 | 0.529183832 | 0.366215987 | −0.577033509 | −0.368760531 | 4.831393557 | 0 |
| HLA-F | 1.444485168 | 0.498379544 | 0.345022265 | 0.964815647 | −2.266991996 | 1.725915777 | 0 |
| NM_001017978 | 1.427877514 | 1.014621852 | 0.710580454 | −0.224603682 | −0.892987895 | 4.428701672 | 0 |
| KCNK5 | 1.427257477 | 0.369812532 | 0.25910709 | −0.394812213 | −0.749799523 | 4.9971541 | 0 |
| IL8 | 1.424232682 | 1.260778239 | 0.885233329 | −0.535254651 | −0.334954193 | 4.457423091 | 0 |
| CD3D | 1.401697296 | 0.861656796 | 0.614723877 | 1.211009854 | −1.848282139 | −1.224101824 | 0 |
| PLEK | 1.38551031 | 0.750323579 | 0.541550339 | 0.981865895 | −2.100476728 | 1.054052326 | 0 |
| APG5L | 1.384929842 | 0.587772228 | 0.424405778 | −0.278484247 | −0.816537229 | 4.502828908 | 0 |
| SQLE | 1.383626911 | 0.653737257 | 0.472480877 | −0.338642445 | −0.702309401 | 4.487435122 | 0 |
| GZMB | 1.379929401 | 0.739799625 | 0.536114112 | 1.079652963 | −2.071884119 | 0.350557635 | 0 |
| ZHX2 | 1.376300562 | 0.287249368 | 0.208711219 | 0.50111541 | 0.572212714 | −5.052438767 | 0 |
| UGCGL1 | 1.367351443 | 0.701077992 | 0.512726992 | 0.267117328 | −1.550775554 | 3.616509448 | 0 |
| CCL13 | 1.366564939 | 0.880917161 | 0.644621515 | 0.8504668 | −2.023072531 | 1.605552446 | 0 |
| TRA@ | 1.366557568 | 0.50085836 | 0.366510984 | 1.244423692 | −1.742162482 | −1.792074452 | 0 |
| KYNU | 1.365539913 | 0.665604089 | 0.487429245 | 0.164408952 | −1.428215055 | 3.836595683 | 0 |
| CXCL1 | 1.357886225 | 1.189798831 | 0.876213934 | 0.083818555 | −1.272489428 | 3.806789013 | 0 |
| CD2 | 1.356531047 | 0.68055979 | 0.501691274 | 1.083438527 | −2.037177753 | 0.209085493 | 0 |
| LCN2 | 1.356303178 | 0.731737175 | 0.539508561 | −0.264368096 | −0.787642412 | 4.317066395 | 0 |
| CHML | 1.352302134 | 0.686910876 | 0.507956661 | −0.385098368 | −0.585519137 | 4.37837495 | 0 |
| MYBL1 | 1.340918657 | 0.411743657 | 0.307060876 | −0.344161262 | −0.71496585 | 4.564683716 | 0 |
| HLA-A | 1.314720277 | 0.466733338 | 0.355005811 | 1.026795769 | −2.070850437 | 0.674757718 | 0 |
| AR | 1.314470902 | 0.835694964 | 0.635765282 | −0.127823564 | 1.312965963 | −3.671578177 | 0 |
| LAP3 | 1.314270547 | 0.538577409 | 0.409791889 | 0.825679794 | −2.009088855 | 1.711687387 | 0 |
| MGC40042 | 1.310860337 | 0.680549844 | 0.519162739 | 1.132458911 | −1.819245592 | −0.835810238 | 0 |
| AK055101 | 1.308994366 | 0.255399832 | 0.195111483 | −0.816760287 | 0.105249301 | 4.706066155 | 0 |
| XCL1 | 1.306253472 | 0.629524445 | 0.481931308 | 1.077659641 | −1.940271326 | −0.084567267 | 0 |
| ENST00000327788 | 1.305753027 | 0.336878204 | 0.257995346 | −0.449192145 | −0.532805301 | 4.596529325 | 0 |
| SOAT1 | 1.303264756 | 0.634011261 | 0.48647925 | 0.789856724 | −1.953368038 | 1.744339641 | 0 |
| CYBB | 1.303035498 | 0.419281562 | 0.321772939 | 0.798289135 | −2.028257859 | 1.946684086 | 0 |
| GPR56 | 1.29814667 | 0.52339874 | 0.403189218 | −0.439137768 | −0.46462386 | 4.302375283 | 0 |
| GBP5 | 1.297228148 | 0.952922501 | 0.734583583 | 0.557784675 | −1.7522536 | 2.499397257 | 0 |
| PSCD3 | 1.288650239 | 0.48929034 | 0.379692119 | −1.013643041 | 2.009799321 | −0.548730835 | 0 |
| RUNX3 | 1.280555812 | 0.72811944 | 0.568596412 | 1.063330155 | −1.849592254 | −0.3040333 | 0 |
| OAZIN | 1.275846277 | 0.523984774 | 0.410695852 | 0.229317055 | −1.446624948 | 3.496759081 | 0 |
| IL10RA | 1.271645498 | 0.483330721 | 0.38008291 | 0.957946329 | −1.991469347 | 0.83172854 | 0 |
| C1orf38 | 1.270849802 | 0.50575591 | 0.397966706 | 0.930848623 | −1.980935851 | 0.963920427 | 0 |
| LCP1 | 1.267295788 | 0.494796601 | 0.390434977 | 0.753219791 | −1.924275097 | 1.872572623 | 0 |
| HCST | 1.267203559 | 0.542467197 | 0.428082129 | 1.105519862 | −1.814751757 | −0.68406717 | 0 |
| OIP5 | 1.266812828 | 0.439934365 | 0.347276532 | −0.208239856 | −0.847951867 | 4.174123456 | 0 |
| PLA2G7 | 1.265439077 | 0.864098127 | 0.68284451 | 0.647134832 | −1.792702898 | 2.082953896 | 0 |
| A_24_P828054 | 1.265124443 | 0.304374673 | 0.240588723 | 0.701479255 | 0.017047776 | −4.407133819 | 0 |
| FGF18 | 1.261522942 | 0.878802697 | 0.696620464 | −0.380004332 | 1.555098077 | −2.931306607 | 0 |
| AW205591 | 1.260786756 | 0.479008498 | 0.379928244 | −0.336311101 | −0.618132016 | 4.186777678 | 0 |
| GZMA | 1.258324726 | 0.872917772 | 0.693714233 | 1.086405481 | −1.530244265 | −1.53288348 | 0 |
| AQP9 | 1.257354769 | 0.854455596 | 0.679566036 | 0.00200621 | −1.08885056 | 3.689653404 | 0 |
| CF529502 | 1.25703408 | 0.256724134 | 0.20423005 | 0.452858458 | 0.537939294 | −4.636716041 | 0 |
| CD8A | 1.254531019 | 0.632019797 | 0.503789693 | 1.088787879 | −1.332105176 | −2.221327254 | 0 |
| AI659667 | 1.248507279 | 0.349609608 | 0.280022082 | −0.378639763 | −0.582408204 | 4.327754421 | 0 |
| ASPM | 1.248026426 | 0.534968397 | 0.428651498 | −0.428622885 | −0.427481966 | 4.11090057 | 0 |
| CD3G | 1.243329817 | 0.798347652 | 0.642104485 | 1.078219773 | −1.49467111 | −1.603080821 | 0.684240244 |
| KPNA2 | 1.242427733 | 0.447888975 | 0.360494992 | 0.011475486 | −1.148847806 | 3.834934527 | 0.684240244 |
| NPL | 1.242309753 | 0.448631058 | 0.361126568 | 0.446170973 | −1.669638381 | 2.910510463 | 0.684240244 |
| COL16A1 | 1.241590473 | 0.492428974 | 0.396611431 | 0.008383173 | 1.108064713 | −3.819395698 | 0.684240244 |
| TNFRSF17 | 1.237707521 | 0.763815681 | 0.617121305 | 0.957897115 | −1.840312381 | 0.318099982 | 0.684240244 |
| E2F3 | 1.235217368 | 0.350466714 | 0.283728778 | −0.481326752 | −0.38109893 | 4.279962223 | 0.684240244 |
| FLJ25471 | 1.232701455 | 0.341321696 | 0.276889181 | −0.373257229 | −0.578258972 | 4.280275328 | 0.684240244 |
| LTB | 1.232389886 | 0.693534139 | 0.562755461 | 1.077579392 | −1.471203751 | −1.678899481 | 0.684240244 |
| IL4I1 | 1.232251697 | 0.656708499 | 0.532933734 | 0.172484958 | −1.311275134 | 3.388928714 | 0.684240244 |
| NUDCD1 | 1.231378943 | 0.591720965 | 0.480535231 | 0.191746864 | −1.343664289 | 3.379628024 | 0.684240244 |
| CR594843 | 1.231292471 | 0.475838607 | 0.386454574 | 1.035162935 | −1.8715957 | −0.054584815 | 0.684240244 |
| SRPK1 | 1.229884804 | 0.56550035 | 0.459799445 | −0.04397755 | −1.030514863 | 3.776411344 | 0.684240244 |
| NUDCD1 | 1.229814009 | 0.55505169 | 0.451329783 | 0.185036154 | −1.340175288 | 3.409371824 | 0.684240244 |
| CD44 | 1.226254604 | 0.557290854 | 0.454465861 | 0.277575912 | −1.446593496 | 3.19744723 | 0.684240244 |
| NUDCD1 | 1.225542548 | 0.583479828 | 0.476099201 | 0.181016169 | −1.326045663 | 3.386255004 | 0.684240244 |
| AB209004 | 1.22380395 | 0.697500037 | 0.56994426 | −0.671638542 | 0.113958093 | 3.776701447 | 0.684240244 |
| CENPF | 1.216506148 | 0.631314443 | 0.518957051 | −0.387843085 | −0.451317797 | 3.939107637 | 0.684240244 |
| KIAA0746 | 1.214872758 | 0.673339328 | 0.554246791 | 0.886291512 | −1.837963265 | 0.754067725 | 0.684240244 |
| NUDCD1 | 1.212860412 | 0.547330569 | 0.451272515 | 0.170559717 | −1.30688135 | 3.385926348 | 0.684240244 |
| HDAC2 | 1.210282728 | 0.446175748 | 0.368654148 | −0.514474429 | −0.248166704 | 4.033508255 | 0.684240244 |
| AF287958 | 1.209959225 | 0.406637936 | 0.336075735 | 0.968895543 | −1.913733474 | 0.499541443 | 0.684240244 |
| FAM49B | 1.206598184 | 0.478314683 | 0.396415882 | 0.447742684 | −1.623111821 | 2.742575555 | 0.684240244 |
| NUDCD1 | 1.204548269 | 0.564139575 | 0.468341194 | 0.196549568 | −1.327841323 | 3.296053178 | 0.684240244 |
| LCP2 | 1.202317419 | 0.438478877 | 0.364694772 | 0.833939163 | −1.884996702 | 1.238565976 | 0.684240244 |
| SCRG1 | 1.202078879 | 0.971012671 | 0.807777833 | −0.007886136 | −1.017433411 | 3.508167639 | 0.684240244 |
| HCLS1 | 1.200371706 | 0.488730831 | 0.407149576 | 1.052716118 | −1.727122965 | −0.654621851 | 0.684240244 |
| HCAP-G | 1.200269965 | 0.631758084 | 0.526346657 | −0.374334312 | −0.458972404 | 3.881378904 | 0.684240244 |
| AK024292 | 1.195715688 | 0.684340049 | 0.57232673 | −0.082799581 | −0.924465516 | 3.656540156 | 0.684240244 |
| CLEC7A | 1.195278601 | 0.659835267 | 0.552034703 | 0.742930865 | −1.784884246 | 1.462435075 | 0.684240244 |
| NUDCD1 | 1.194549078 | 0.596256785 | 0.499148002 | 0.200347358 | −1.31756587 | 3.237570338 | 0.684240244 |
| MPEG1 | 1.193960342 | 0.552707727 | 0.46291967 | 0.704860267 | −1.786444797 | 1.703778653 | 0.684240244 |
| GIMAP4 | 1.193252833 | 0.7739695 | 0.648621548 | 0.755314451 | −1.770500146 | 1.336750899 | 0.684240244 |
| CTSC | 1.192479775 | 0.469075565 | 0.393361443 | 0.743932551 | −1.826994659 | 1.599400025 | 0.684240244 |
| NUDCD1 | 1.192245833 | 0.588370034 | 0.493497245 | 0.187957462 | −1.301372501 | 3.259330239 | 0.684240244 |
| NUDCD1 | 1.18842407 | 0.593475597 | 0.499380324 | 0.184484036 | −1.292619976 | 3.251106896 | 0.73542357 |
| SFRP2 | 1.185966056 | 0.350199765 | 0.295286499 | 0.143749899 | 0.898375331 | −3.945725496 | 0.73542357 |
| LPXN | 1.1855095 | 0.499412149 | 0.421263726 | 0.943641157 | −1.8198777 | 0.337009009 | 0.73542357 |

TABLE 6
Multivariate Cox regression, including hypoxia, for
overall survival in the complete NKI data[29].

| | | | | Lower | Upper |
|---|---|---|---|---|---|
| | | | Relative | 95% | 95% |
| Variable | Significance | | Risk | C.I. | C.I. |
| Stroma Predictor | 6.60E−03 | ** | 2.549 | 5.008 | 1.297 |
| (mixed outcome) | | | | | |
| Stroma Predictor | 7.80E−03 | ** | 2.646 | 5.42 | 1.292 |
| (poor outcome) | | | | | |
| Age (<40 years) | 1.50E−02 | * | 0.95 | 0.99 | 0.912 |
| Nodes Positive (>4) | 2.80E−02 | * | 2.22 | 4.54 | 1.093 |
| Nodes Positive (0) | 5.10E−01 | | 1.2 | 2.064 | 0.698 |
| 70 genes (poor | 3.50E−03 | ** | 3.7 | 9.213 | 1.55 |
| outcome) | | | | | |
| Hypoxia (hypoxic | 2.20E−02 | * | 1.98 | 3.552 | 1.106 |
| reponse) | | | | | |
| HER2 (positive) | 3.50E−02 | * | 1.8 | 3.116 | 1.044 |
| ER (positive) | 7.10E−01 | | 0.9 | 1.564 | 0.52 |
| Wound Signature | 3.20E−02 | * | 0.303 | 0.902 | 0.102 |
| (intermediate) | | | | | |
| Wound Signature | 5.40E−02 | | 0.531 | 1.012 | 0.278 |
| (quiescent) | | | | | |
| Grade (poorly | 7.90E−01 | | 0.93 | 1.6 | 0.541 |
| differentiated) | | | | | |
| Grade (well | 1.10E−01 | | 0.407 | 1.229 | 0.135 |
| differentiated) | | | | | |

*, p < 0.05;
**, p < 0.01

TABLE 7
Material and methods for immunohistochemistry

| | Antibody | | | |
|---|---|---|---|---|
| | CD8 | CD3z | OPN | CD31 |
| Tissue | FFPE | FFPE | FFPE | FFAF |
| Antigen | 0.001M | 0.001M | none | none |
| retrieval | EDTA, pH 8.0 | citrate | | |
| | | buffer, | | |
| | | pH 8.0 | | |
| Peroxidase | Dako | Dako | 3% H2O2 in | Dako |
| blocking | Blocking | Blocking | methanol | Blocking |
| | Solution | Solution | | Solution |
| | (K4006) | (K4006) | | (K4006) |
| Antigen | 10% horse | 10% horse | none | none |
| blocking | serum in TBS | serum in PBS | | |
| Primary | N1592 (Dako | sc-1239 | ab8448 | M0823 |
| antibody | Cytomation) | (Santa Cruz | (Abcam) | (Dako |
| | | Biotechnology) | | Cytomation) |
| Secondary | K4006 (Dako | K4006 (Dako | PK-6102 | K4006 (Dako |
| antibody | Cytomation) | Cytomation) | (Vector | Cytomation) |
| | | | Laboratories) | |
| Visualization | Standard | Standard | Standard | Standard |
| | DAB staining | DAB staining | DAB staining | DAB staining |

TABLE 8

| Sequences of PCR Primers and | | | |
|---|---|---|---|
| Universal Probe Library Probes Used | | | |
| in Quantitative PCR | | | |
| | GenBank | Amplicon | |
| Gene | Accession # | size (bp) | Primer sequences and probes |

| | | | | |
|---|---|---|---|---|
| CXCL1 | BC011976.1 | 73 | Forward primer | aagcaaatggccaatgagat |
| | | | | (SEQ ID NO: 1) |
| | | | Reverse primer | atctaaacagttacaaaacagatgtgc |
| | | | | (SEQ ID NO: 2) |
| | | | Universal Probe Library probe: #8 | |
| | | | (Roche, cat. no. 04685067001) | |
| VGLL1 | NM_016267 | 75 | Forward primer | catcgatacctgcagcatctt |
| | | | | (SEQ ID NO: 3) |
| | | | Reverse primer | tgtcttgctgccgtgtctta |
| | | | | (SEQ ID NO: 4) |
| | | | Universal Probe Library probe #25 | |
| | | | (Roche, cat. no. 04686993001) | |
| LCP1 | BC010271 | 70 | Forward primer | ttggcagtggactcagaaag |
| | | | | (SEQ ID NO: 5) |
| | | | Reverse primer | Tcagaagcagcaaaaatactgg |
| | | | | (SEQ ID NO: 6) |
| | | | Universal Probe Library probe #23 | |
| | | | (Roche, cat. no. 04686977001) | |
| ADM | NM_001124 | 125 | Forward primer | ccgcgtggaatgtgagtgtg |
| | | | | (SEQ ID NO: 7) |
| | | | Reverse primer | Tgctgttcgcatatcacccattt |
| | | | | (SEQ ID NO: 8) |
| CD8A | BC025715 | 233 | Forward primer | tgcctgcccattggagagaa |
| | | | | (SEQ ID NO: 9) |
| | | | Reverse primer | Ttttcggatgctgtttacccatt |
| | | | | (SEQ ID NO: 10) |
| SPP1 | NM_001040060 | 102 | Forward primer | tgttgtgattatctttttgtggtgtga |
| | | | | (SEQ ID NO: 11) |
| | | | Reverse primer | gctggacaaccgtgggaaaa |
| | | | | (SEQ ID NO: 12) |

TABLE 9
List of 26 genes of optimal predictor

| Gene Symbol | Gene Name |
|---|---|
| GZMA | granzyme A (granzyme 1; cytotoxic T-lymphocyte-associated serine esterase 3) |
| CD8A | CD8a (CD8; MAL; p32; Leu2) |
| TRBV5-4 | T cell receptor beta variable 5-4 (TRBV54; TCRBV5S4; TCRBV5S6A3N2T) |
| CD52 | CD52 (CDW52; CAMPATH-1 antigen) |
| CD3Z | CD247 (CD3H; CD3Q; CD3Z; TCRZ; CD3-ZETA) |
| CD48 | CD48 (BCM1; BLAST; hCD48; mCD48; BLAST1; SLAMF2; MEM-102) |
| PLEK | pleckstrin (P47; FLJ27168) |
| RUNX3 | runt-related transcription factor 3 (AML2; CBFA3; PEBP2aC; FLJ34510; |
| | MGC16070) |
| GIMAP5 | GTPase, IMAP family member 5 (IAN4; IAN5; IMAP3; hIAN5; HIMAP3; IAN4L1; |
| | FLJ11296) |
| LCP1 | lymphocyte cytosolic protein 1 (L-plastin) (CP64; PLS2; LC64P; FLJ25423; |
| | FLJ26114; FLJ39956; L-PLASTIN; DKFZp781A23186) |
| F2RL2 | coagulation factor II (thrombin) receptor-like 2 (PAR3) |
| SLC40A1 | solute carrier family 40 (iron-regulated transporter), member 1 (FPN1; HFE4; |
| | MTP1; IREG1; MST079; MSTP079; SLC11A3) |
| FRZB | frizzled-related protein (FRE; FZRB; hFIZ; FRITZ; FRP-3; FRZB1; SFRP3; |
| | SRFP3; FRZB-1; FRZB-PEN) |
| RAI2 | retinoic acid induced 2 |
| HOXA10 | homeobox A10 (PL; HOX1; HOX1H; HOX1.8; MGC12859) |
| AL359052 | Homo sapiens mRNA full length insert cDNA clone EUROIMAGE 1968422 OR |
| | integrin, beta-like 1 (with EGF-like repeat domains) (OSCP; TIED) |
| OGN | osteoglycin (mimecan; OIF; SLRR3A; DKFZP586P2421) |
| C21orf34 | chromosome 21 open reading frame 34 (C21orf35; FLJ38295; hypothetical |
| | protein LOC388815) |
| ADRA2A | adrenergic, alpha-2A-, receptor (ADRA2; ADRAR; ZNF32; ADRA2R; |
| | ALPHA2AAR) |
| CXCL14 | chemokine (C-X-C motif) ligand 14 (KS1; Kec; BMAC; BRAK; NJAC; MIP-2g; |
| | SCYB14; MGC10687; bolekine) |
| SPP1 | secreted phosphoprotein 1 (osteopontin, bone sialoprotein I, early T- |
| | lymphocyte activation 1) (OPN; BNSP; BSPI; ETA-1; MGC110940) |
| HRASLS | HRAS-like suppressor (A-C1; HSD28; HRASLS1; H-REV107) |
| VGLL1 | vestigial like 1 (Drosophila) (TDU; VGL1) |
| ADM | adrenomedullin (AM) |
| AK055101 | Homo sapiens cDNA FLJ30539 fis, clone BRAWH2001255 |
| THC2394165 | syntrophin, gamma 2 (SYN5; G2SYN; MGC133174 ) |

TABLE 10
List of other predictor gene sets
Any number of genes from each of the following three groups:

| Group | Other | Group | Other | Group | Other |
|---|---|---|---|---|---|
| 1 | Names | 2 | names | 3 | Names |
| GZMA | | F2RL2 | | SPP1 | |
| CD8A | | SLC40A1 | | HRASLS | |
| TRBC1 | | FRZB | | VGLL1 | |
| CD52 | | RAI2 | | ADM | |
| CD3Z | | HOXA10 | | AK055101 | Homo sapiens |
| | | | | | cDNA FLJ30539 fis, |
| | | | | | clone |
| | | | | | BRAWH2001255 gi\| |
| | | | | | 16549759\|dbj\| |
| | | | | | AK055101.1\| |
| | | | | | [16549759] |
| CD48 | | AL359052 | Integrin | THC2394165 | SNTG2 |
| | | | beta like | | |
| | | | 1 | | |
| PLEK | | OGN | | | |
| RUNX3 | | C21orf34 | | | |
| GIMAP5 | | ADRA2A | | | |
| LCP1 | | CXCL14 | | | |

TABLE 11
Genes Overlapping With NKI and Wang et al. data sets.

| | |
|---|---|
| NKI (Van de Vijver et al.) | CD8 |
| | GZMA |
| | RUNX3 |
| | LCP1 |
| | CD48 |
| | PLEK |
| | CD3Z |
| | HOXA10 |
| | ADRA2A |
| | F2RL2 |
| | OGN |
| | RAI2 |
| | FRZB |
| | ADM |
| | SPP1 |
| Wang et al. | RUNX3 |
| | PLEK |
| | GZMA |
| | CD48 |
| | CD3Z |
| | GIMAP5 |
| | CD8A |
| | LCP1 |
| | CD52 |
| | CXCL14 |
| | RAI2 |
| | ADRA2A |
| | OGN |
| | FRZB |
| | HOXA10 |
| | ADM |
| | SPP1 |
| | VGLL1 |
| | HRASLS |

### Example 2

### SDPP Integration with Other Predictors

### Integration of Multiple Predictors

The independent predictions of the 70-gene predictor, wound response signature, hypoxia signature, and our SDPP in the NKI data set were combined, to construct a Bayes' classifier of metastasis. The structure of the classifier was to condition metastasis on the output of wound response, 70-gene, hypoxia, and the SDPP. In order to compare the good and poor-outcome classes of each predictor, cases predicted as mixed or intermediate outcome for the SDPP and wound signatures, respectively, were removed for training. Posterior probabilities of metastasis were then estimated given different combinations of each predictor, including the case where information from a predictor was not used.

Bayesian Network Integrating the Hypoxia, 70 Gene, and Wound Signatures with the SDPP.

The structure and parameters of the Bayesian network that integrates the 70 gene, wound response, and hypoxic transcriptional response with the SDPP, as well as survival, metastasis, estrogen receptor status, and HER2 receptor status was learned from the NKI data set. The network was used to make inferences regarding posterior probabilities conditional on a variety of events including observation of individual signatures in isolation and in combinations.

### Results

Having demonstrated that the SDPP was an independent prognostic predictor, the SDPP was tested for whether it adds predictive value to known predictors and signatures. For this a graphical modeling approach was applied (See Materials and Methods, FIG. 9 f). Using the NKI data set, and predictions from the 70-gene predictor, wound response and hypoxia signatures, and the SDPP, a Bayes' classifier of metastasis was constructed. From this analysis, the 70-gene, hypoxia, and wound response signatures each have a posterior probability of metastasis of less than 50%, whereas the SDPP has a posterior probability of metastasis of 56% (FIG. 5g) demonstrating the increased accuracy of the SDPP. Notably, combining the SDPP with any of the predictors improves the prediction of metastasis beyond that of any of the predictors alone, and beyond any combination of predictors that does not include the SDPP (FIG. 9 g, black points). Comparable improvements were observed when the SDPP is combined with other predictors to predict good outcome (FIG. 9 g, grey points). These results demonstrate an interaction between the biological processes underlying the predictors and highlight the increased prognostic power to be derived from an integrative approach.

### Discussion

The SDPP provides a significant improvement in predictive accuracy when applied in combination with the other signatures/predictors (FIG. 9 g). Thus, distinct gene expression signatures in breast tumor stroma reflect different clinical outcomes, which are not restricted to a specific clinical subtype. The stroma-specific signature presented here, alone or in combination with other molecular prognostic predictors, promises to improve molecular classification and prediction of outcome in breast cancer, specifically for the identification of patients that may benefit from adjuvant or aggressive therapies. Additional information is derived from the SDPP, beyond that provided by classical clinical risk factors or published molecular signatures. This, in combination with the improved accuracy provided by a combinatorial approach, clearly highlights the need to fully integrate all aspects of the tumor microenvironment into prognostic prediction and may suggest future avenues of investigation for the development of additional targeted therapeutic modalities.

### Example 3

### Identification of Genes Differentially Expressed in Tumor Associated Stroma of Other Cancers for Predicting Outcome

### Description of Samples

Tissue samples comprising tumor associated stroma and normal stroma from cancer patients such as colon cancer patients or lung cancer patients are subjected to laser capture microdissection (LCM). Recurrence (local or distant) is determined by examination of medical records following diagnosis. Poor outcome is defined as alive with disease or dead of disease as of the time of the latest follow-up.

### LCM, RNA Isolation and Microarray Hybridization

Regions of tumor-associated and normal stroma are identified by a clinical pathologist prior to microdissection. LCM, sample isolation and preparations, as well as microarray hybridization, are carried out as previously described²³. Normal stroma is harvested at least 2 mm away from the tumor margins. Each RNA sample is hybridized on Agilent 44K whole human genome microarrays in a dye-swap replication design; samples or a subset of samples are optionally hybridized in duplicate, triplicate, and/or quadruplicate. Normalization and model fitting is performed as previously described^{23,24}.

Identification of a Tumor Stroma Subtype Associated with Recurrence and Poor Outcome

A LIMMA²⁵ model to the patient-matched tumor-associated vs. normal stroma data is applied, and the top 200 most variable genes across all patients, which are also differentially expressed in at least 3 patients (p<1e-5) are identified. This approach excluded genes that co-vary between tumor and normal stroma. Tumor stroma is clustered using these genes and the significance of clusters is assessed by bootstrapping (1000 bootstrap iterations) using the pvclust package²⁶. Each cluster is tested for association with known predictors of outcome that depend on the cancer type and may include lymph node, and p53 status, as well as grade, recurrence, and outcome, using a c² association test.

### Identification of Genes Differentially Expressed Between the Tumor Stroma Subtypes

Pair-wise class distinction is used to identify genes differentially expressed between the poor outcome, mixed outcome, and good outcome associated stroma subtypes previously defined by class discovery. The expression profile of the outcome-associated tumor stroma subtypes is derived from the union of differentially expressed genes from SAM²⁷ (multiclass comparison, q-value<0.01), and LIMMA (intersection of top 200 differentially expressed for each comparison, ranked by fold change FDR adjusted p-value<0.01).

### Predictor Construction and Evaluation

Logistic regression is used to score and rank each gene in the expression profile, based on its significance in estimating binomial recurrence in a model including gene expression level, and other predictors such as lymph node status. This model ensures that the predictive strength of a gene is not confounded with other predictor status.

Naïve Bayes' classifiers are trained to predict prognosis using the ranked gene expression profile of the recurrence-positive stroma cluster. Each classifier is trained on an incrementally larger set of genes from the ranked list, and then evaluated using cross validation runs by randomly splitting the data into testing and training sets of equal size, Receiver-operator-characteristic (ROC) curves are generated for each classifier, and classifiers are compared using their area under the curve (AUC). The optimal predictor is selected to maximize the AUC, and trained on all the data. The performance of the SDPP in tumor stroma to its performance in tumor epithelium, normal stroma, and normal epithelium is compared using the AUC.

### Gene Ontology (GO) Analysis

Genes differentially expressed in each stroma subtype are cross-referenced against GO annotations²⁸ to identify overrepresented GO categories using a test against the hypergeometric distribution, using a significance threshold of p≦0.05.

### Immunohistochemistry

Expression of proteins corresponding to selected members is validated by immunohistochemistry, using sections from formalin-fixed paraffin-embedded blocks. Slides are then scanned using an Aperio ScanScope XT (Aperio Technologies, Vista, Calif.) with a 20× objective and images extracted using the ImageScope image viewer (Aperio Technologies).

### Q-RT-PCR

Amplified RNA (aRNA) prepared from microdissected tissues is used as a template for RT-Qt PCR validation using a LightCycler instrument (Roche Applied Science) as per the manufacturer's instructions. aRNA is initially reverse transcribed using AMV reverse transcriptase (Roche). All primers and probe sequences are designed within 300 by of the 3′-end. The crossing point is automatically calculated using the LightCycler 3.5 software and determined from the second derivative maximum on the PCR amplification curve. Transcript quantification is performed by comparison with standard curves generated from dilution series of cDNA from pooled connective aRNA (crossing point vs. log initial RNA amount). Melt curve analyses confirmed that single products are amplified. Agarose gel electrophoresis is used to establish that PCR products are of the predicted length.

### Example 4

### Materials and Methods

### Description of Samples

Laser capture microdissection was used to isolate normal stroma and epithelium as well as tumor stroma and epithelium from each sample whenever possible. Tissue samples from 91 patients were microdissected. The cohort of 91 patients was composed of 68 patients with invasive ductal carcinoma (IDC), 1 patient with invasive lobular carcinoma (ILC), and 17 healthy donors who had undergone breast reduction surgery. From this cohort, the following samples were obtained: 53 samples of tumor stroma from IDC, 63 samples of tumor epithelium from IDC, 47 samples of normal stroma, of which nine were from breast reduction samples, 57 samples of normal epithelium (15 breast reduction cases), one sample of tumor epithelium from ILC, and three samples of tumor epithelium from lymph nodes. In total, 226 distinct tissue samples were obtained by microdissection from the 91 patients.

Each sample was hybridized as a dye-swap: 219 samples were hybridized in duplicate, three in triplicate, and four in quadruplicate. In total, 463 arrays were obtained. After normalization and model fitting, a microarray dataset of 226 distinct expression experiments was produced. The following summarizes the results of the tumor stroma analysis.

Identification of a Tumor Stroma Subtype Associated with Recurrence and Poor Outcome

A LIMMA model was fitted to the patient-matched tumor vs normal stroma data and identified the top 200 most variable genes across all patients, which were differentially expressed in at least 3 patients. Tumor stroma was clustered using these genes and the significance of the clusters was assessed using the bootstrap. Each cluster was tested for association with ER, PR, lymph node, Her2, p53 status, grade, recurrence, and outcome.

### Identification of Genes Differentially Expressed in the Poor Outcome Tumor Stroma Subtype

The genes differentially expressed between poor outcome tumor stroma subtype and the remaining tumor stroma samples were identified using the LIMMA (top 200 genes ranked by fold change, fdr adjusted p-value<0.01) and SAM (q-value<0.01) approaches to class distinction. The set union of these approaches was used to derive the expression profile of tumor stroma with poor outcome.

Logistic regression was used to identify those genes from the expression profile that were predictive of recurrence or poor outcome. A multivariate model that included lymph node status, estrogen receptor status, progesterone receptor status, and Her2 receptor status was fitted. Genes that are significantly associated with recurrence or outcome (p<0.05) in the multivariate logistic regression model were identified.

### Evaluation of the Prognostic Predictor by Cross Validation

A naïve bayes classifier was trained to predict prognosis based on the genes identified as significant by the logistic regression model in tumor stroma. The classifier was evaluated under cross validation, by splitting the data randomly into a testing and a training set of equal size. ROC curves and the area under the curves were generated for the classifier, and were compared to ROC curves for a classifier trained on tumor epithelium data, using the same features.

Comparison with Publicly Available Breast Cancer Datasets

Publicly available breast cancer data was downloaded¹⁸ and the data clustered using the genes identified as associated with recurrence or outcome in tumor stroma. The two clusters of samples defined by these genes were treated as a categorical variable in Cox proportional hazard survival analysis, and tested for significance against survival, time to metastasis, local recurrence and regional recurrence.

### Immunohistochemistry

Genes identified as significantly associated with poor outcome tumor stroma were validated by immunohistochemistry on paraffin sections of breast tissue.

### Results

Class Discovery Identifies a Tumor Stroma Subtype Associated with Poor Outcome

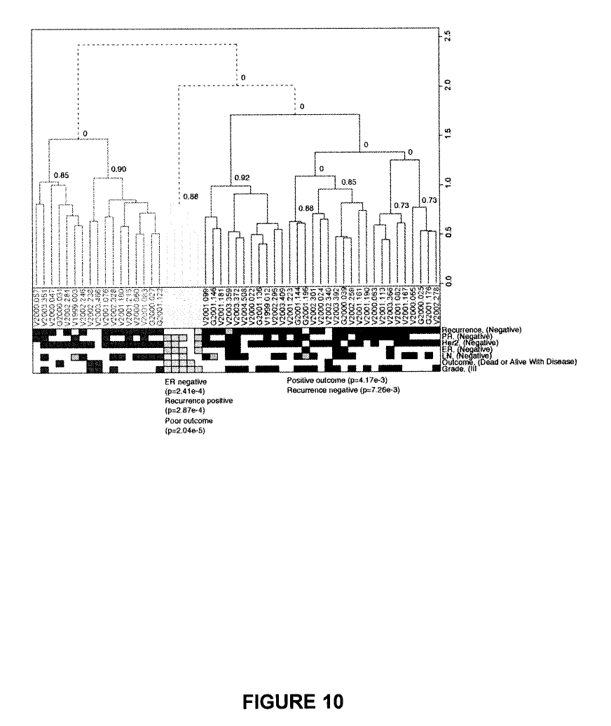

A cluster of tumor stroma that is associated with patients with poor outcome (alive with disease or dead of disease, p=2.04e-5, c² test for association), and positive for recurrence (p=2.87e-4, c² test for association) was identified (FIG. 10). This cluster of patients was not detected when tumor epithelium was analyzed in the same manner.

### Genes Defining the Poor Outcome Tumor Stroma Cluster

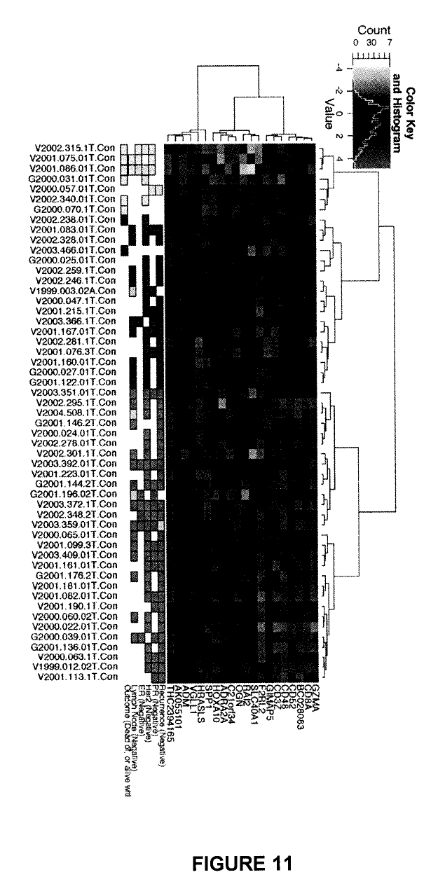

The genes differentially expressed between the poor outcome tumor stroma subcluster and the remaining subclusters of tumor stroma were identified. Seventy-two (72) genes were identified as differentially expressed between the clusters (q-value<0.01) using SAM. The top 200 genes differentially expressed between the clusters were selected using LIMMA (ranked by fold change, fdr adjusted p<0.01). Twenty (20) genes were identified as significantly associated with recurrence or outcome in the logistic regression model and were used to cluster the tumor stroma expression data (FIG. 11).

### Evaluation of the Prognostic Predictor

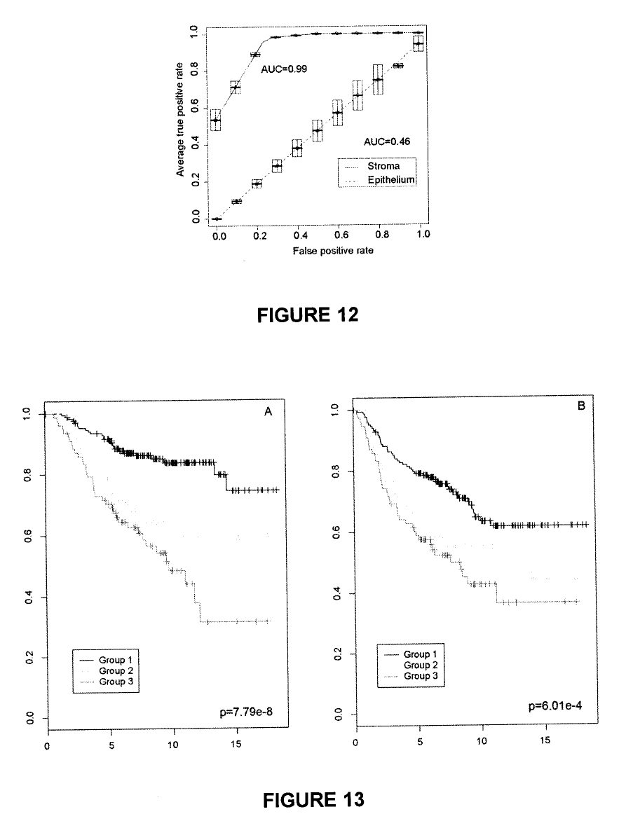

The 20 genes identified by logistic regression were used to build a naïve bayes classifier of outcome. The data was randomly split into a testing and a training set, and the performance of the classifier was evaluated. ROC curves show that the classifier performed well under cross-validation, with an AUC of 0.99. These same were poor predictors of outcome in tumor epithelium, with an AUC of 0.46 (FIG. 12), and also a poor predictor in normal stroma (AUC=0.45).

### Predictor Performance in Publicly Available Data Sets

The derived predictor was tested using a publicly available data set. Clustering the data set using the predictor revealed three groups of samples. Kaplan-Meier survival analysis showed that group 3 had significantly poorer overall survival (p=4.1e-7, log rank test) and shorter recurrence free survival (p=7.8e-4, log rank test) than the other two groups combined (FIG. 13A). Similarly, group 1 had significantly improved overall survival (p=4.87e-8, log rank test) and longer recurrence free survival (p=2.21e-4) than groups 2 and 3 combined (FIG. 13B). The difference in overall survival and recurrence free survival between all three groups is also significant (p=7.79e-8, p=6.01e-4, respectively, log rank test).

Cox proportional hazards regression showed that the overall survival for group 3 was significantly decreased in a multivariate analysis including ER status, tumor size, lymph node involvement, mastectomy, grade, age, chemotherapy, hormonal therapy, as well as the wound signature predictor, and the 70 gene predictor.

### Predictor Gene Expression in Tumor Stroma

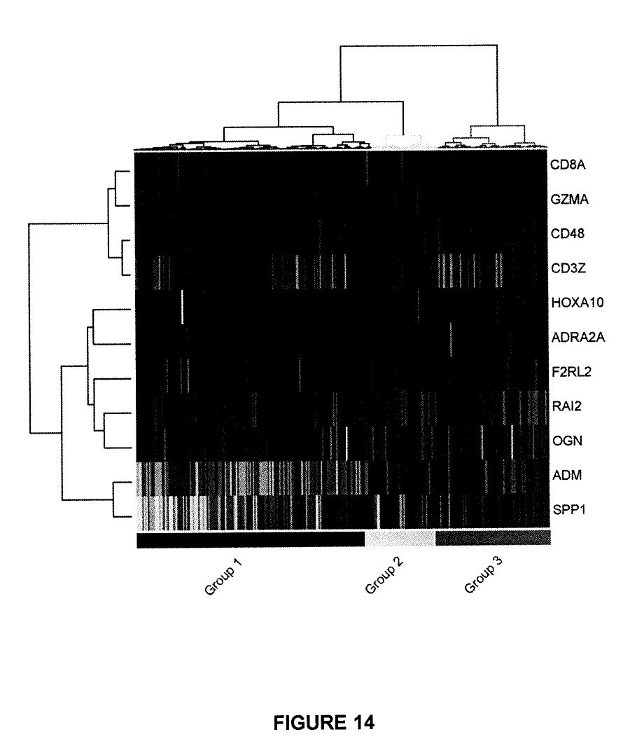

The cluster of stroma associated with poor outcome expressed elevated levels of adrenomedullin, a pro-angiogenic factor, as well as decreased levels of HOXA10, a transcription factor whose expression in breast cancer cells has been shown to lead to a decrease in invasive phenotype⁵²⁵³. This cluster also shows a decrease in a number of proteins often downregulated in gastric tumors, including OGN and HRASLS⁵⁴⁵⁵. Furthermore, this group shows a decrease in expression of a number of T-cell markers and natural killer cell markers, including granzyme A, CD8A, and CD3Z. There is also decreased expression of CD48, a B-cell activation marker, as well as decreased expression of CD52, a lymphocyte and monocyte antigen important in the complement-mediated immune response. Interestingly, the combination of elevated angiogenic factors and decreased T-cell markers is predictive of poor prognosis in both the presently generated dataset and the publicly available breast cancer dataset (FIG. 11, FIG. 14).

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Parkin, D. M., Bray, F., Ferlay, J. & Pisani, P. Global cancer statistics, 2002. CA Cancer J Clin 55, 74-108 (2005).
2. Glas, A. M. et al. Converting a breast cancer microarray signature into a high-throughput diagnostic test. BMC Genomics 7, 278 (2006).
3. van't Veer, L. J. et al. Gene expression profiling predicts clinical outcome of breast cancer. Nature 415, 530-6 (2002).
4. Sorlie, T. et al. Repeated observation of breast tumor subtypes in independent gene expression data sets. Proc Natl Acad Sci USA 100, 8418-8423 (2003).
5. Cobleigh, M. A. et al. Tumor gene expression and prognosis in breast cancer patients with 10 or more positive lymph nodes. Clin Cancer Res 11, 8623-31 (2005).
6. West, R. B. et al. Determination of stromal signatures in breast carcinoma. PLoS Biol 3, e187 (2005).
7. Allinen, M. et al. Molecular characterization of the tumor microenvironment in breast cancer. Cancer Cell 6, 17-32 (2004).
8. Ma, X.-J. et al. Gene expression profiles of human breast cancer progression. Proc Natl Acad Sci USA 100, 5974-9 (2003).
9. Sgroi, D. C. et al. In vivo gene expression profile analysis of human breast cancer progression. Cancer Res 59, 5656-61 (1999).
10. Huber, M. A. et al. Expression of stromal cell markers in distinct compartments of human skin cancers. J Cutan Pathol 33, 145-55 (2006).
11. Iyer, V. R. et al. The transcriptional program in the response of human fibroblasts to serum. Science 283, 83-7 (1999).
12. Wang, Y. et al. Gene-expression profiles to predict distant metastasis of lymph-node-negative primary breast cancer. Lancet 365, 671-9 (2005).
13. Micke, P. & Ostman, A. Tumour-stroma interaction: cancer-associated fibroblasts as novel targets in anti-cancer therapy? Lung Cancer 45 Suppl 2, S163-75 (2004).
14. Bissell, M. J. & Radisky, D. Putting tumours in context. Nat Rev Cancer 1, 46-54 (2001).
15. Dunn, G. P., Koebel, C. M. & Schreiber, R. D. Interferons, immunity and cancer immunoediting. Nat Rev Immunol 6, 836-48 (2006).
16. Smyth, M. J., Dunn, G. P. & Schreiber, R. D. Cancer immunosurveillance and immunoediting: the roles of immunity in suppressing tumor development and shaping tumor immunogenicity. Adv Immunol 90, 1-50 (2006).
17. Strausberg, R. L. Tumor microenvironments, the immune system and cancer survival. Genome Biol 6, 211 (2005).
18. van de Vijver, M. J. et al. A gene-expression signature as a predictor of survival in breast cancer. N Engl J Med 347, 1999-2009 (2002).
19. Chi, J. T. et al. Gene Expression Programs in Response to Hypoxia: Cell Type Specificity and Prognostic Significance in Human Cancers. PLoS Med 3, e47 (2006).
20. Chang, H. Y. et al. Robustness, scalability, and integration of a wound-response gene expression signature in predicting breast cancer survival. Proc Natl Acad Sci USA 102, 3738-3743 (2005).
21. Bhowmick, N. A., Neilson, E. G. & Moses, H. L. Stromal fibroblasts in cancer initiation and progression. Nature 432, 332-7 (2004).
22. Bhowmick, N. A. et al. TGF-beta signaling in fibroblasts modulates the oncogenic potential of adjacent epithelia. Science 303, 848-51 (2004).
23. Finak, G. et al. Gene expression signatures of morphologically normal breast tissue identify basal-like tumors. Breast Cancer Res 8, R58 (2006).
24. Finak, G. et al. BIAS: Bioinformatics Integrated Application Software. Bioinformatics 21, 1745-6 (2005).
25. Smyth, G. K. Linear models and empirical bayes methods for assessing differential expression in microarray experiments. Stat Appl Genet Mol Biol 3, Article 3 (2004).
26. Suzuki, R. & Shimodaira, H. Pvclust: an R package for assessing the uncertainty in hierarchical clustering. Bioinformatics 22, 1540-2 (2006).
27. Tusher, V. G., Tibshirani, R. & Chu, G. Significance analysis of microarrays applied to the ionizing radiation response. Proc Natl Acad Sci USA 98, 5116-21 (2001).
28. Ashburner, M. et al. Gene ontology: tool for the unification of biology. The Gene Ontology Consortium. Nat Genet 25, 25-9 (2000).
29. Miller, L. D. et al. An expression signature for p53 status in human breast cancer predicts mutation status, transcriptional effects, and patient survival. Proc Natl Acad Sci USA 102, 13550-5 (2005).
30. Perou, C. M. et al. Molecular portraits of human breast tumours. Nature 406, 747-752 (2000).
31. Guidi, A. J. et al. Association of angiogenesis in lymph node metastases with outcome of breast cancer. J Natl Cancer Inst 92, 486-92 (2000).
32. Gruber, G. et al. Hypoxia-inducible factor 1 alpha in high-risk breast cancer: an independent prognostic parameter? Breast Cancer Res 6, R191-8 (2004).
33. Ribatti, D., Conconi, M. T. & Nussdorfer, G. G. Nonclassic endogenous regulators of angiogenesis. Pharmacol Rev 59, 185-205 (2007).
34. Bobrovnikova-Marjon, E. V., Marjon, P. L., Barbash, O., Vander Jagt, D. L. & Abcouwer, S. F. Expression of angiogenic factors vascular endothelial growth factor and interleukin-8/CXCL8 is highly responsive to ambient glutamine availability: role of nuclear factor-kappaB and activating protein-1. Cancer Res 64, 4858-69 (2004).
35. Mohsenin, A., Burdick, M. D., Molina, J. G., Keane, M. P. & Blackburn, M. R. Enhanced CXCL1 production and angiogenesis in adenosine-mediated lung disease. Faseb J (2007).
36. Gupta, G. P. et al. Mediators of vascular remodelling co-opted for sequential steps in lung metastasis. Nature 446, 765-70 (2007).
37. Nuyten, D. S. & van de Vijver, M. J. Gene expression signatures to predict the development of metastasis in breast cancer. Breast Dis 26, 149-56 (2006).
38. Pages, F. et al. Effector memory T cells, early metastasis, and survival in colorectal cancer. N Engl J Med 353, 2654-66 (2005).
39. Singh, V. K., Mehrotra, S. & Agarwal, S. S. The paradigm of Th1 and Th2 cytokines: its relevance to autoimmunity and allergy. Immunol Res 20, 147-61 (1999).
40. Sica, A., Schioppa, T., Mantovani, A. & Allavena, P. Tumour-associated macrophages are a distinct M2 polarised population promoting tumour progression: potential targets of anti-cancer therapy. Eur J Cancer 42, 717-27 (2006).
41. Condeelis, J. & Pollard, J. W. Macrophages: obligate partners for tumor cell migration, invasion, and metastasis. Cell 124, 263-6 (2006).
42. Pollard, J. W. Tumour-educated macrophages promote tumour progression and metastasis. Nat Rev Cancer 4, 71-8 (2004).
43. Murdoch, C., Giannoudis, A. & Lewis, C. E. Mechanisms regulating the recruitment of macrophages into hypoxic areas of tumors and other ischemic tissues. Blood 104, 2224-34 (2004).
44. Deonarine, K. et al. Gene expression profiling of cutaneous wound healing. J Transl Med 5, 11 (2007).
45. Parker, B. S. et al. Alterations in vascular gene expression in invasive breast carcinoma. Cancer Res 64, 7857-66 (2004).
46. Uzzan, B., Nicolas, P., Cucherat, M. & Perret, G. Y. Microvessel density as a prognostic factor in women with breast cancer: a systematic review of the literature and meta-analysis. Cancer Res 64, 2941-55 (2004).
47. Rudland, P. S. et al. Prognostic significance of the metastasis-associated protein osteopontin in human breast cancer. Cancer Res 62, 3417-27 (2002).
48. Chia, S. K., Speers, C. H., Bryce, C. J., Hayes, M. M. & Olivotto, I. A. Ten-year outcomes in a population-based cohort of node-negative, lymphatic, and vascular invasion-negative early breast cancers without adjuvant systemic therapies. J Clin Oncol 22, 1630-7 (2004).
49. Fitzgibbons, P. L. et al. Prognostic factors in breast cancer. College of American Pathologists Consensus Statement 1999. Arch Pathol Lab Med 124, 966-78 (2000).
50. Spiridon, C. I., Guinn, S. & Vitetta, E. S. A comparison of the in vitro and in vivo activities of IgG and F(ab′)2 fragments of a mixture of three monoclonal anti-Her-2 antibodies. Clin Cancer Res 10, 3542-51 (2004).
51. van't Veer, L. J. et al. Expression profiling predicts outcome in breast cancer. Breast Cancer Res 5, 57-8 (2003).
52. Chu, M. C., Selam, F. B. & Taylor, H. S. HOXA10 regulates p53 expression and matrigel invasion in human breast cancer cells. Cancer Biol Ther 3, 568-72 (2004).
53. Kawakami, Y. [Adrenomedullin antagonist suppresses in vivo proliferation of cancer cells in SCID mice via angiogenesis inhibition]. Hokkaido Igaku Zasshi 80, 575-83 (2005).
54. Imura, M. et al. Methylation and expression analysis of 15 genes and three normally-methylated genes in 13 Ovarian cancer cell lines. Cancer Lett 241, 213-220 (2006).
55. Tasheva, E. S., Maki, C. G., Conrad, A. H. & Conrad, G. W. Transcriptional activation of bovine mimecan by p53 through an intronic DNA-binding site. Biochim Biophys Acta 1517, 333-8 (2001).

1. (canceled)

2. A method for predicting disease outcome in a breast cancer patient, for predicting recurrence in a breast cancer patient, or for diagnosing a breast cancer sub-type in a subject having breast cancer comprising:
a) obtaining an expression level of at least 3 genes a stroma derived prognostic predictor (SDPP) gene set in a sample of the patient, wherein at least one of the genes is selected from the group consisting of TRBV5-4, C21orf34, AK055101 and THC2394165;
b) comparing the expression level of the genes in the sample to a reference expression profile for the genes in the SDPP gene set; and
c) predicting a good, mixed or poor prognosis disease outcome, recurrence or diagnosing breast cancer subtype in the patient;
wherein the reference expression profile of the at least 3 genes in the SDPP gene set correlates with a disease outcome, recurrence or breast cancer subtype class, the class being either a good prognosis, a mixed prognosis or a poor prognosis wherein a good prognosis predicts recurrence free survival of the patient, a poor prognosis predicts recurrence or non-survival, and a mixed prognosis predicts either recurrence free survival, or recurrence and/or non-survival, or wherein a good prognosis predicts a breast cancer subtype associated with recurrence free survival, a poor prognosis predicts a breast cancer subtype with recurrence or non-survival, and mixed prognosis predicts a breast cancer subtype with either recurrence free survival, or recurrence and/or non-survival and wherein disease outcome is predicted according to the statistical probability of falling within the class defined by the reference expression profile of the at least 3 genes in the SDPP gene set.

3-4. (canceled)

5. The method of claim 1 for diagnosing poor prognosis breast cancer comprising:
a) obtaining an expression level of at least 3 genes of a SDPP gene set in a sample of a subject, wherein at least one of the genes is selected from the group consisting of TRBV5-4, C21orf34, AK055101 and THC2394165; and
b) comparing the expression level of the genes to a reference expression profile of corresponding genes in the SDPP gene set;
wherein the reference expression profile of the at least 3 genes in the SDPP gene set correlates with a poor prognosis class and wherein the subject is diagnosed to have the poor prognosis according to the statistical probability of falling within the poor prognosis class.

6-9. (canceled)

10. The method of claim 1 further comprising displaying or outputting a result of one or more steps to a user, a computer readable storage medium, a monitor, or a computer that is part of a network.

11. The method of claim 1 wherein the SDPP gene set comprises at least 3 genes selected from Tables 3, 4, 5, 9, 10, or 11.

12-15. (canceled)

16. The method of claim 11 wherein the SDPP gene set comprises the genes from Table 9 or 11 or a group of genes from Table 10.

17-20. (canceled)

21. The method of claim 1 wherein the gene expression level is detected using a microarray chip or a PCR method.

22. The method of claim 21 wherein the microarray chip also detects one or more genes selected from the group consisting of the Wang, NKI, wound signature or 70 gene predictor data sets.

23-25. (canceled)

26. The method of claim 21 wherein the PCR method comprises using one or more primers selected from the group consisting of SEQ ID NOS:1-12.

27. The method of claim 1 where the gene expression level is obtained by detecting the level of a plurality of polypeptides, wherein each of the plurality of polypeptides corresponds to a gene in the SDPP gene set.

28. (canceled)

29. The method of claim 27 wherein each polypeptide is detected using an antibody that specifically binds to the polypeptide, by performing immunohistochemical analysis on the sample, or by performing an ELISA assay.

30-31. (canceled)

32. The method of claim 1 wherein the breast cancer is selected from the group consisting of a HER2 positive or HER2 negative, ER positive or ER negative, PR positive or PR negative, node positive or node negative, high grade or low grade, basal-like or luminal like, or any combination of thereof, breast cancer.

33-35. (canceled)

36. The method of claim 1 wherein the sample is selected from a group consisting of a tumor biopsy sample, a frozen tissue sample, a cell sample, a paraffin embedded sample and a tumor associated stroma tissue sample.

37-41. (canceled)

42. A method of monitoring effectiveness of a treatment in a breast cancer patient comprising:
a) obtaining an expression level for at least 3 genes of an SDPP gene set in a first sample of a patient, wherein the first sample is taken before or after the start of the treatment, wherein at least one of the genes is selected from the group consisting of TRBV5-4, C21orf34, AK055101 and THC2394165;
b) obtaining an expression level for at least 3 genes of a SDPP gene set in a second sample of a patient, wherein the second sample is taken subsequent to the first sample and after at least one treatment;
c) comparing the expression levels of the genes in the first and second sample to the reference expression profile of the genes in the SDPP gene set; and
d) determining the disease outcome class for the first and second sample;
wherein a change in the outcome class of the second sample indicates a decreased probability of poor prognosis and indicates the treatment is effective.

43-45. (canceled)

46. An array comprising for each gene in a plurality of genes, the plurality of genes being at least 3 of the genes listed in Tables 3-5 or 9-11, one or more polynucleotide probes complementary and hybridizable to a coding sequence in the gene, wherein at least one of the genes is selected from the group consisting of TRBV5-4, C21orf34, AK055101 and THC2394165.

47-48. (canceled)

49. The array of claim 46 comprising a substrate comprising a plurality of addresses, wherein each address has disposed thereon the polynucleotide probe that can specifically bind a gene of one or more SDPP gene sets of Tables 3-5 and/or 9-11.

50-51. (canceled)

52. A composition comprising:
two or more isolated nucleic acid sequences, wherein each isolated nucleic acid sequence hybridizes to:
a) a RNA product of a gene of a SDPP gene set; and/or
b) a nucleic acid sequence complementary to a);
or two or more isolated antibodies, wherein each antibody binds a polypeptide product of a gene of a SDPP gene set;
wherein the composition is used to measure the expression level of 2 or more genes of a SDPP gene set selected from Tables 3-5 and/or 9-11, and wherein at least one of the genes is selected from the group consisting of TRBV5-4, C21orf34, AK055101 and THC2394165.

53-56. (canceled)

57. A kit for classifying a breast cancer comprising:
two or more isolated nucleic acids wherein each isolated nucleic acid sequence hybridizes to:
a) a RNA product of a gene of a SDPP gene set; and/or
b) a nucleic acid sequence complementary to a);
or two or more isolated antibodies, wherein each antibody binds a polypeptide product of a gene of a SDPP gene set;
or an array according to claim 46; and
instructions for use, wherein the two or more isolated nucleic acids, or the two or more antibodies or the array detect expression levels of at least 3 genes of a SDPP gene set.

58-64. (canceled)

65. A computer system comprising:
a) a processor; and
b) a memory coupled to the processor and encoding one or more programs, wherein the one or more programs cause the processor to carry out the method of claim 1, steps (b) and (c).

66. (canceled)

67. A computer implemented stroma derived prognostic predictor (SDPP) system for predicting disease outcome in a breast cancer patient comprising:
a) values corresponding to at least 3 genes of a SDPP gene set, wherein at least one of the genes is selected from the group consisting of TRBV5-4, C21orf34, AK055101 and THC2394165;
b) a weighting for each gene in the SDPP gene set according to a reference expression profile for each gene in the SDPP gene set, wherein the weighting is associated with disease outcome; and
c) a means for receiving values corresponding to an expression level for each gene of the SDPP gene set in a patient sample;
wherein the SDPP predicts disease outcome in a breast cancer patient by comparing the reference expression profile and weighting for at least 3 genes in the SDPP gene set to an expression level of a corresponding gene in a sample from a breast cancer patient.

68-69. (canceled)

70. The computer system according to claim 65—comprising:
a) a database including records comprising the reference expression profiles of a plurality of genes in Tables 3-54 and/or 9-11 and associated clinical outcome weighting;
b) a user interface capable of receiving a selection of gene expression levels of at least 3 genes in Tables 3-54 and/or 9-11 for use in comparing to the tumor associated gene expression profiles in the database;
c) an output that displays a prediction of clinical outcome according to the expression levels of the at least 3 genes.

71. A computer readable medium on which is stored a database capable of configuring a computer to respond to queries based on records belonging to the database, each of the records comprising:
a) a value that identifies a gene of a SDPP gene set and/or a gene reference expression profile of a SDPP gene set;
b) a value that identifies the probability of a clinical outcome associated with the gene and/or gene reference expression profile.

72-75. (canceled)
